SEQUENCE LISTING (58 sequences)

SEQ ID NO: 1 - RNA, 60 nt, Artificial Sequence
  Synthetic polynucleotide
gaaccccuaccaacuggucgggguuugaaacagcagcagcagcagcagcagcauuuuuuu

SEQ ID NO: 2 - RNA, 60 nt, Artificial Sequence
  Synthetic polynucleotide
gaaccccuaccaacuggucgggguuugaaacacaaaaguaagauucacuuucauuuuuuu

SEQ ID NO: 3 - RNA, 60 nt, Artificial Sequence
  Synthetic polynucleotide
gaaccccuaccaacuggucgggguuugaaacgagaauucuaguagggauguaguuuuuuu

SEQ ID NO: 4 - RNA, 60 nt, Artificial Sequence
  Synthetic polynucleotide
gaaccccuaccaacuggucgggguuugaaacuuucuuccacacaaccaaccaguuuuuuu

SEQ ID NO: 5 - RNA, 60 nt, Artificial Sequence
  Synthetic polynucleotide
gaaccccuaccaacuggucgggguuugaaacaaugugagcaccuuccuucuuuuuuuuuu

SEQ ID NO: 6 - RNA, 60 nt, Artificial Sequence
  Synthetic polynucleotide
gaaccccuaccaacuggucgggguuugaaacggcugcaguuaagguuuucuuguuuuuuu

SEQ ID NO: 7 - RNA, 60 nt, Artificial Sequence
  Synthetic polynucleotide
gaaccccuaccaacuggucgggguuugaaacauaucgccuggauccugagccauuuuuuu

SEQ ID NO: 8 - RNA, 96 nt, Artificial Sequence
  Synthetic polynucleotide
gaaccccuaccaacuggucgggguuugaaacgagaauucuaguagggauguagcaaguaaaccccuaccaacuggucgggguuugaaacuuuuuuu

SEQ ID NO: 9 - RNA, 96 nt, Artificial Sequence
  Synthetic polynucleotide
gaaccccuaccaacuggucgggguuugaaacauaucgccuggauccugagccacaaguaaaccccuaccaacuggucgggguuugaaacuuuuuuu

SEQ ID NO: 10 - RNA, 234 nt, Artificial Sequence
  Synthetic polynucleotide
gaaccccuaccaacuggucgggguuugaaacacaaaaguaagauucacuuucacaaguaaaccccuaccaacuggucgggguuugaaacgagaauucuaguagggauguagcaaguaaaccccuaccaacuggucgggguuugaaacuuucuuccacacaaccaaccagcaaguaaaccccuaccaacuggucgggguuugaaacauaucgccuggauccugagccauuuuuuu

SEQ ID NO: 11 - RNA, 130 nt, Artificial Sequence
  Synthetic polynucleotide
gaaccccuaccaacuggucgggguuugaaacgagaauucuaguagggauguagcgaauacgagggucuccagauggccaacaugaggaucacccaugucugcagggccagaucucguauucguuuuuuuu

SEQ ID NO: 12 - RNA, 217 nt, Artificial Sequence
  Synthetic polynucleotide
gaaccccuaccaacuggucgggguuugaaacgagaauucuaguagggauguagcgaauacgagggucuccagaugcguacaccaucaggguacgcagaugcguacaccaucaggguacgcagaugcguacaccaucaggguacgcagaugcguacaccaucaggguacgcagaugcguacaccaucaggguacgcagaucucguauucguuuuuuuu

SEQ ID NO: 13 - protein, 1000 aa, Artificial Sequence
  Synthetic polynucleotide
MSPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYA

SEQ ID NO: 14 - protein, 7 aa, Artificial Sequence
  Synthetic polynucleotide
PKKKRKV

SEQ ID NO: 15 - protein, 24 aa, Artificial Sequence
  Synthetic polynucleotide
DPKKKRKVDPKKKRKVDPKKKRKV

SEQ ID NO: 16 - protein, 5 aa, Artificial Sequence
  Synthetic polynucleotide
GGGGS

SEQ ID NO: 17 - protein, 15 aa, Artificial Sequence
  Synthetic polynucleotide
GGGGSGGGGSGGGGS

SEQ ID NO: 18 - protein, 23 aa, Artificial Sequence
  Synthetic polynucleotide
MDYKDHDGDYKDHDIDYKDDDDK

SEQ ID NO: 19 - protein, 9 aa, Artificial Sequence
  Synthetic polynucleotide
YPYDVPDYA

SEQ ID NO: 20 - protein, 1353 aa, Artificial Sequence
  Synthetic polynucleotide
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 21 - protein, 1341 aa, Artificial Sequence
  Synthetic polynucleotide
MLLQPAPCAPSAGFPRPLAAPGAMHGSQKDTTFTKIFVGGLPYHTTDASLRKYFEGFGDIEEAVVITDRQTGKSRGYGFVTMADRAAAERACKDPNPIIDGRKANVNLAYLGAKPRSLQTGFAIGVQQLHPTLIQRTYGLTPHYIYPPAIVQPSVVIPAAPVPSLSSPYIEYTPASPAYAQYPPATYDQYPYAASPATAASFVGYSYPAAVPQALSAAAPAGTTFVQYQAPQLQPDRMQNVIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMDYKDHDGDYKDHDIDYKDDDDK

SEQ ID NO: 22 - protein, 1339 aa, Artificial Sequence
  Synthetic polynucleotide
MDYKDHDGDYKDHDIDYKDDDDKIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMLLQPAPCAPSAGFPRPLAAPGAMHGSQKDTTFTKIFVGGLPYHTTDASLRKYFEGFGDIEEAVVITDRQTGKSRGYGFVTMADRAAAERACKDPNPIIDGRKANVNLAYLGAKPRSLQTGFAIGVQQLHPTLIQRTYGLTPHYIYPPAIVQPSVVIPAAPVPSLSSPYIEYTPASPAYAQYPPATYDQYPYAASPATAASFVGYSYPAAVPQALSAAAPAGTTFVQYQAPQLQPDRMQ

SEQ ID NO: 23 - protein, 486 aa, Artificial Sequence
  Synthetic polynucleotide
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRAMASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSAGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 24 - protein, 1317 aa, Artificial Sequence
  Synthetic polynucleotide
MDYKDHDGDYKDHDIDYKDDDDKIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPARDSKSQAPGQPGASQWGSRVVPNAANGWAGQPPPTWQQGYGPQGMWVPAGQAIGGYGPPPAGRGAPPPPPPFTSYIVSTPPGGFPPPQGFPQGYGAPPQFSFGYGPPPPPPDQFAPPGVPPPPATPGAAPLAFPPPPSQAAPDMSKPPTAQPDFPYGQYAGYGQDLSGFGQGFSDPSQQPPSYGGPSVPGSGGPPAGGSGFGRGQNHNVQGFHPYRR

SEQ ID NO: 25 - protein, 1575 aa, Artificial Sequence
  Synthetic polynucleotide
MGMSDFDEFERQLNENKQERDKENRHRKRSHSRSRSRDRKRRSRSRDRRNRDQRSASRDRRRRSKPLTRGAKEEHGGLIRSPRHEKKKKVRKYWDVPPPGFEHITPMQYKAMQAAGQIPATALLPTMTPDGLAVTPTPVPVVGSQMTRQARRLYVGNIPFGITEEAMMDFFNAQMRLGGLTQAPGNPVLAVQINQDKNFAFLEFRSVDETTQAMAFDGIIFQGQSLKIRRPHDYQPLPGMSENPSVYVPGVVSTVVPDSAHKLFIGGLPNYLNDDQVKELLTSFGPLKAFNLVKDSATGLSKGYAFCEYVDINVTDQAIAGLNGMQLGDKKLLVQRASVGAKNATLSTINQTPVTLQVPGLMSSQVQMGGHPTEVLCLMNMVLPEELLDDEEYEEIVEDVRDECSKYGLVKSIEIPRPVDGVEVPGCGKIFVEFTSVFDCQKAMQGLTGRKFANRVVVTKYCDPDSYHRRDFWNVIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMDYKDHDGDYKDHDIDYKDDDDK

SEQ ID NO: 26 - protein, 1342 aa, Artificial Sequence
  Synthetic polynucleotide
MAEYLASIFGTEKDKVNCSFYFKIGACRHGDRCSRLHNKPTFSQTIALLNIYRNPQNSSQSADGLRCAVSDVEMQEHYDEFFEEVFTEMEEKYGEVEEMNVCDNLGDHLVGNVYVKFRREEDAEKAVIDLNNRWFNGQPLHAELSPVTDFREACCRQYEMGECTRGGFCNFMHLKPISRELRRELYGRRRKKHRSRSRSRERRSRSRDRGRGGGGGGGGGGGGRERDRRRSRDRERSGRFNVIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMDYKDHDGDYKDHDIDYKDDDDK

SEQ ID NO: 27 - protein, 1571 aa, Artificial Sequence
  Synthetic polynucleotide
MDYKDHDGDYKDHDIDYKDDDDKIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMSDFDEFERQLNENKQERDKENRHRKRSHSRSRSRDRKRRSRSRDRRNRDQRSASRDRRRRSKPLTRGAKEEHGGLIRSPRHEKKKKVRKYWDVPPPGFEHITPMQYKAMQAAGQIPATALLPTMTPDGLAVTPTPVPVVGSQMTRQARRLYVGNIPFGITEEAMMDFFNAQMRLGGLTQAPGNPVLAVQINQDKNFAFLEFRSVDETTQAMAFDGIIFQGQSLKIRRPHDYQPLPGMSENPSVYVPGVVSTVVPDSAHKLFIGGLPNYLNDDQVKELLTSFGPLKAFNLVKDSATGLSKGYAFCEYVDINVTDQAIAGLNGMQLGDKKLLVQRASVGAKNATLSTINQMPVTLQVPGLMSSQVQMGGHPTEVLCLMNMVLPEELLDDEEYEEIVEDVRDECSKYGLVKSIEIPRPVDGVEVPGCGKIFVEFTSVFDCQKAMQGLTGRKFANRVVVTKYCDPDSYHRRDFW

SEQ ID NO: 28 - protein, 1340 aa, Artificial Sequence
  Synthetic polynucleotide
MDYKDHDGDYKDHDIDYKDDDDKIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMAEYLASIFGTEKDKVNCSFYFKIGACRHGDRCSRLHNKPTFSQTILIQNIYRNPQNSAQTADGSHCAVSDVEMQEHYDEFFEEVFTEMEEKYGEVEEMNVCDNLGDHLVGNVYVKFRREEDAEKAVIDLNNRWFNGQPIHAELSPVTDFREACCRQYEMGECTRGGFCNFMHLKPISRELRRELYGRRRKKHRSRSRSRERRSRSRDRGRGGGGGGGGGGGGRERDRRRSRDRERSGRF

SEQ ID NO: 29 - protein, 1226 aa, Artificial Sequence
  Synthetic polynucleotide
MGGGSSGGGQISYASRGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLENVIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMDYKDHDGDYKDHDIDYKDDDDK

SEQ ID NO: 30 - protein, 1223 aa, Artificial Sequence
  Synthetic polynucleotide
MDYKDHDGDYKDHDIDYKDDDDKIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAGGGSSGGGQISYASRGGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 31 - protein, 457 aa, Artificial Sequence
  Synthetic polynucleotide
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRAMEMWHEGLEEASRLYFGERNVKGMFEVLEPLHAMMERGPQTLKETSFNQAYGRDLMEAQEWCRKYMKSGNVKDLTQAWDLYYHVFRRISKQQISYASRGGGSSGGGGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 32 - protein, 445 aa, Artificial Sequence
  Synthetic polynucleotide
MLLQPAPCAPSAGFPRPLAAPGAMHGSQKDTTFTKIFVGGLPYHTTDASLRKYFEGFGDIEEAVVITDRQTGKSRGYGFVTMADRAAAERACKDPNPIIDGRKANVNLAYLGAKPRSLQTGFAIGVQQLHPTLIQRTYGLTPHYIYPPAIVQPSVVIPAAPVPSLSSPYIEYTPASPAYAQYPPATYDQYPYAASPATAASFVGYSYPAAVPQALSAAAPAGTTFVQYQAPQLQPDRMQNVIDGGGGSDPKKKRKVDPKKKRKVDPKKKRKVGSTGSRNDGGGGSGGGGSGGGGSGRAMEMWHEGLEEASRLYFGERNVKGMFEVLEPLHAMMERGPQTLKETSFNQAYGRDLMEAQEWCRKYMKSGNVKDLTQAWDLYYHVFRRISKQQISYASRGGGSSGGGGGGGSGGGGSGGGGSGPAMDYKDHDGDYKDHDIDYKDDDDK

SEQ ID NO: 33 - protein, 443 aa, Artificial Sequence
  Synthetic polynucleotide
MDYKDHDGDYKDHDIDYKDDDDKIDGGGGSDPKKKRKVDPKKKRKVDPKKKRKVGSTGSRNDGGGGSGGGGSGGGGSGRAMEMWHEGLEEASRLYFGERNVKGMFEVLEPLHAMMERGPQTLKETSFNQAYGRDLMEAQEWCRKYMKSGNVKDLTQAWDLYYHVFRRISKQQISYASRGGGSSGGGGGGGSGGGGSGGGGSGPAMLLQPAPCAPSAGFPRPLAAPGAMHGSQKDTTFTKIFVGGLPYHTTDASLRKYFEGFGDIEEAVVITDRQTGKSRGYGFVTMADRAAAERACKDPNPIIDGRKANVNLAYLGAKPRSLQTGFAIGVQQLHPTLIQRTYGLTPHYIYPPAIVQPSVVIPAAPVPSLSSPYIEYTPASPAYAQYPPATYDQYPYAASPATAASFVGYSYPAAVPQALSAAAPAGTTFVQYQAPQLQPDRMQ

SEQ ID NO: 34 - DNA, 4584 bp, Artificial Sequence
  Synthetic polynucleotide
ctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgatcaaatccgctcccggcggatttgtcctactcaggagagcgttcaccgacaaacaacagataaaacgaaaggcccagtattccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcaccggtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgggtcttctcgacctgcagactggctgtgtataagggagcctgacatttatattccccagaacatcaggttaatggcgtttttgatgtcattttcgcggtggctgagatcagccacttcttccccgataacggacaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgacacgaaaaacatattctcaataaaccctttagggaaataggccaggttttcaccgtaacacgccacatcttgcgaatatatgtgtagaaactgccggaaatcgtcgtggtattcactccagagcgatgaaaaggtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgcgaagaccttttttttggcgcgccttaattaagaattcgacccagctttcttgtacaaagttggcattataaaaaataattgctcatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgcctcctctagaccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtt

SEQ ID NO: 35 - DNA, 4433 bp, Artificial Sequence
  Synthetic polynucleotide
attgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcagctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgggtcttctcgacctgcagactggctgtgtataagggagcctgacatttatattccccagaacatcaggttaatggcgtttttgatgtcattttcgcggtggctgagatcagccacttcttccccgataacggacaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgacacgaaaaacatattctcaataaaccctttagggaaataggccaggttttcaccgtaacacgccacatcttgcgaatatatgtgtagaaactgccggaaatcgtcgtggtattcactccagagcgatgaaaaggtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgcgaagacaacgaatacgagggtctccagatggccaacatgaggatcacccatgtctgcagggccagatctcgtattcgttttttttggcgcgccgaattctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttag

SEQ ID NO: 36 - DNA, 4520 bp, Artificial Sequence
  Synthetic polynucleotide
attgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcagctagcgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgggtcttctcgacctgcagactggctgtgtataagggagcctgacatttatattccccagaacatcaggttaatggcgtttttgatgtcattttcgcggtggctgagatcagccacttcttccccgataacggacaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatccagatctttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgacacgaaaaacatattctcaataaaccctttagggaaataggccaggttttcaccgtaacacgccacatcttgcgaatatatgtgtagaaactgccggaaatcgtcgtggtattcactccagagcgatgaaaaggtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgcgaagacaacgaatacgagggtctccagatgcgtacaccatcagggtacgcagatgcgtacaccatcagggtacgcagatgcgtacaccatcagggtacgcagatgcgtacaccatcagggtacgcagatgcgtacaccatcagggtacgcagatctcgtattcgttttttttggcgcgccgaattctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttag

SEQ ID NO: 37 - DNA, 10820 bp, Artificial Sequence
  Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagataattcccccaccacctcccatatgtccagattctcttgatgatgctgatgctttgggaagtatgttaatttcatggtacatgagtggctatcatactggctattatatggtaagtaatcactcagcatcttttcctgacaatttttttgtagttatgtgactttgttttgtaaatttataaaatactacttgcttctctctttatattactaaaaaataaaaataaaaaaatacaactgtctgaggcttaaattactcttgcattgtccctaagtataattttagttaattttaaaaagctttcatgctattgttagattattttgattatacacttttgaattgaaattatactttttctaaataatgttttaatctctgatttgaaattgattgtagggaatggaaaagatgggataatttttcataaatgaaaaatgaaattcttttttttttttttttttttttgagacggagtcttgctctgttgcccaggctggagtgcaatggcgtgatcttggctcacagcaagctctgcctcctggattcacgccattctcctgcctcagcctcagaggtagctgggactacaggtgcctgccaccacgcctgtctaattttttgtatttttttgtaaagacagggtttcactgtgttagccaggatggtctcaatctcctgaccccgtgatccacccgcctcggccttccaagagaaatgaaatttttttaatgcacaaagatctggggtaatgtgtaccacattgaaccttggggagtatggcttcaaacttgtcactttatacgttagtctcctacggacatgttctattgtattttagtcagaacatttaaaattattttattttattttattttttttttttttttgagacggagtctcgctctgtcacccaggctggagtacagtggcgcagtctcggctcactgcaagctccgcctcccgggttcacgccattctcctgcctcagcctctccgagtagctgggactacaggcgcccgccaccacgcccggctaatttttttttatttttagtagagacggggtttcaccgtggtctcgatctcctgacctcgtgatccacccgcctcggcctcccaaagtgctgggattacaagcgtgagccaccgcgcccggcctaaaattatttttaaaagtaagctcttgtgccctgctaaaattatgatgtgatattgtaggcacttgtatttttagtaaattaatatagaagaaacaactgacttaaaggtgtatgtttttaaatgtatcatctgtgtgtgcccccattaatattcttatttaaaagttaaggccagacatggtggcttacaactgtaatcccaacagtttgtgaggccgaggcaggcagatcacttgaggtcaggagtttgagaccagcctggccaacatgatgaaaccttgtctctactaaaaataccaaaaaaaatttagccaggcatggtggcacatgcctgtaatccgagctacttgggaggctgtggcaggaaaattgctttaatctgggaggcagaggttgcagtgagttgagattgtgccactgcactccacccttggtgacagagtgagattccatctcaaaaaaagaaaaaggcctggcacggtggctcacacctataatcccagtactttgggaggtagaggcaggtggatcacttgaggttaggagttcaggaccagcctggccaacatggtgactactccatttctactaaatacacaaaacttagcccagtggcgggcagttgtaatcccagctacttgagaggttgaggcaggagaatcacttgaacctgggaggcagaggttgcagtgagccgagatcacaccgctgcactctagcctggccaacagagtgagaatttgcggagggaaaaaaaagtcacgcttcagttgttgtagtataaccttggtatattgtatgtatcatgaattcctcattttaatgaccaaaaagtaataaatcaacagcttgtaatttgttttgagatcagttatctgactgtaacactgtaggcttttgtgttttttaaattatgaaatatttgaaaaaaatacataatgtatatataaagtattggtataatttatgttctaaataactttcttgagaaataattcacatggtgtgcagtttacctttgaaagtatacaagttggctgggcacaatggctcacgcctgtaatcccagcactttgggaggccagggcaggtggatcacgaggtcaggagatcgagaccatcctggctaacatggtgaaaccccgtctctactaaaagtacaaaaacaaattagccgggcatgttggcgggcaccttttgtcccagctgctcgggaggctgaggcaggagagtggcgtgaacccaggaggtggagcttgcagtgagccgagattgtgccagtgcactccagcctgggcgacagagcgagactctgtctcaaaaaataaaataaaaaagaaagtatacaagtcagtggttttggttttcagttatgcaaccatcactacaatttaagaacattttcatcaccccaaaaagaaaccctgttaccttcattttccccagccctaggcagtcagtacactttctgtctctatgaatttgtctattttagatattatatataaacggaattatacgatatgtggtcttttgtgtctggcttctttcacttagcatgctattttcaagattcatccatgctgtagaatgcaccagtactgcattccttcttattgctgaatattctgttgtttggttatatcacattttatccattcatcagttcatggacatttaggttgtttttatttttgggctataatgaataatgttgctatgaacattcgtttgtgttctttttgtttttttggttttttgggttttttttgttttgtttttgtttttgagacagtcttgctctgtctcctaagctggagtgcagtggcatgatcttggcttactgcaagctctgcctcccgggttcacaccattctcctgcctcagcccgacaagtagctgggactacaggcgtgtgccaccatgcacggctaattttttgtatttttagtagagatggggtttcaccgtgttagccaggatggtctcgatctcctgacctcgtgatctgcctgcctaggcctcccaaagtgctgggattacaggcgtgagccactgcacctggccttaagtgtttttaatacgtcattgccttaagctaacaattcttaacctttgttctactgaagccacgtggttgagataggctctgagtctagcttttaacctctatctttttgtcttagaaatctaagcagaatgcaaatgactaagaataatgttgttgaaataacataaaataggttataactttgatactcattagtaacaaatctttcaatacatcttacggtctgttaggtgtagattagtaatgaagtgggaagccactgcaagctagtatacatgtagggaaagatagaaagcattgaagccagaagagagacagaggacatttgggctagatctgacaagaaaaacaaatgttttagtattaatttttgactttaaattttttttttatttagtgaatactggtgtttaatggtctcattttaataagtatgacacaggtagtttaaggtcatatattttatttgatgaaaataaggtataggccgggcacggtggctcacacctgtaatcccagcactttgggaggccgaggcaggcggatcacctgaggtcgggagttagagactagcctcaacatggagaaaccccgtctctactaaaaaaaatacaaaattaggcgggcgtggtggtgcatgcctgtaatcccagctactcaggaggctgaggcaggagaattgcttgaacctgggaggtggaggttgcggtgagccgagatcacctcattgcactccagcctgggcaacaagagcaaaactccatctcaaaaaaaaaaaaataaggtataagcgggctcaggaacatcattggacatactgaaagaagaaaaatcagctgggcgcagtggctcacgccggtaatcccaacactttgggaggccaaggcaggcgaatcacctgaagtcgggagttccagatcagcctgaccaacatggagaaaccctgtctctactaaaaatacaaaactagccgggcatggtggcgcatgcctgtaatcccagctacttgggaggctgaggcaggagaattgcttgaaccgagaaggcggaggttgcggtgagccaagattgcaccattgcactccagcctgggcaacaagagcgaaactccgtctcaaaaaaaaaaggaagaaaaatatttttttaaattaattagtttatttattttttaagatggagttttgccctgtcacccaggctggggtgcaatggtgcaatctcggctcactgcaacctccgcctcctgggttcaagtgattctcctgcctcagcttcccgagtagctgtgattacagccatatgccaccacgcccagccagttttgtgttttgttttgttttttgttttttttttttgagagggtgtcttgctctgtcccccaagctggagtgcagcggcgcgatcttggctcactgcaagctctgcctcccaggttcacaccattctcttgcctcagcctcccgagtagctgggactacaggtgcccgccaccacacccggctaatttttttgtgtttttagtagagatggggtttcactgtgttagccaggatggtctcgatctcctgaccttttgatccacccgcctcagcctccccaagtgctgggattataggcgtgagccactgtgcccggcctagtcttgtatttttagtagagtcgggatttctccatgttggtcaggctgttctccaaatccgacctcaggtgatccgcccgccttggcctccaaaagtgcaaggcaaggcattacaggcatgagccactgtgaccggcaatgtttttaaattttttacatttaaattttattttttagagaccaggtctcactctattgctcaggctggagtgcaagggcacattcacagctcactgcagccttgacctccagggctcaagcagtcctctcacctcagtttcccgagtagctgggactacagtgataatgccactgcacctggctaatttttatttttatttatttatttttttttgagacagagtcttgctctgtcacccaggctggagtgcagtggtgtaaatctcagctcactgcagcctccgcctcctgggttcaagtgattctcctgcctcaacctcccaagtagctgggattagaggtccccaccaccatgcctggctaattttttgtactttcagtagaaacggggttttgccatgttggccaggctgttctcgaactcctgagctcaggtgatccaactgtctcggcctcccaaagtgctgggattacaggcgtgagccactgtgcctagcctgagccaccacgccggcctaatttttaaattttttgtagagacagggtctcattatgttgcccagggtggtgtcaagctccaggtctcaagtgatccccctacctccgcctcccaaagttgtgggattgtaggcatgagccactgcaagaaaaccttaactgcagcctaataattgttttctttgggataacttttaaagtacattaaaagactatcaacttaatttctgatcatattttgttgaataaaataagtaaaatgtcttgtgaaacaaaatgctttttaacatccatataaagctatctatatatagctatctatatctatatagctattttttttaacttcctttattttccttacagggttttagacaaaatcaaaaagaaggaaggtgctcacattccttaaattaaggagtaagtctgccagcattatgaaagtgaatcttacttttgtaaaactttatggtttgtggaaaacaaatgtttttgaacatttaaaaagttcagatgttagaaagttgaaaggttaatgtaaaacaatcaatattaaagaattttgatgccaaaactattagataaaaggttaatctacatccctactagaattctcatacttaactggttggttgtgtggaagaaacatactttcacaataaagagctttaggatatgatgccattttatatcactagtaggcagaccagcagacttttttttattgtgatatgggataacctaggcatactgcactgtacactctgacatatgaagtgctctagtcaagtttaactggtgtccacagaggacatggtttaactggaattcgtcaagcctctggttctaatttctcatttgcaggaaatgctggcatagagcagcactaaatgacaccactaaagaaacgatcagacagatctggaatgtgaagcgttatagaagataactggcctcatttcttcaaaatatcaagtgttgggaaagaaaaaaggaagtggaatgggtaactcttcttgattaaaagttatgtaataaccaaatgcaatgtgaaatattttactggactctattttgaaaaaccatctgtaaaagactgaggtgggggtgggaggccagcacggtggtgaggcagttgagaaaatttgaatgtggattagattttgaatgatattggataattattggtaattttatgagctgtgagaagggtgttgtagtttataaaagactgtcttaatttgcatacttaagcatttaggaatgaagtgttagagtgtcttaaaatgtttcaaatggtttaacaaaatgtatgtgaggcgtatgtgcccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 38 - DNA, 8139 bp, Artificial Sequence
  Synthetic polynucleotide
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttccatggattacaaggatgacgatgacaagggggtacctgccccaaaaaaaaaacgcaaagtggaggacccagtaccaggatctagaggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggatccaggcgatatccggaagaattcaggtagttactgcacctttctttgttccatctctccacctctgctgtgaataaatcgcgggtcggtgtgtcctgtgcctttccctgcttgggaaacgctttcctttcattctttcacttctctgctgctttttgcgctctccccatcctgctgtgccaacctgctctcagttctgtgctttctgtcttccatcccaacacacccctgggttgctgtcttctttctcctttcttcctctcttgctgtgggaccaaacgtctcctgcaggacctgcgggctctgacagaggactctcgtgggggtactgctccctccagtggaaaaatgctccagcagtgtcatgcaggagatttatgccatacagttttgctctctgctgcatggaggggagcagcagaagtcgatctcccccactctggggtccccctcgaggggggcacagctggggagggaacaagggacaaaaccaggagggggctccgagtccttggatttattccccctcatccatgccttaccttcaggtaagggcctgaacagagccctttacttcctgcttctttctcccatagctccctctccttcgggtctcctggactcagtgccacggttgtcccattctgggggtctgtagggagccagcaggagctgcggccgtcctactgaccctgtccttattgcacaggtcaggaggatcaggaggacgaggaggaagaggagaccggtgtgcgctcctccaagaacgtcatcaaggagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagaccatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggccgccaccacctgttcctgtagaccgcggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 39 - DNA, 20 bp, Artificial Sequence
  Synthetic polynucleotide
gctaacgcagtcagtgcttc

SEQ ID NO: 40 - DNA, 21 bp, Artificial Sequence
  Synthetic polynucleotide
gtatcttatcatgtctgctcg

SEQ ID NO: 41 - DNA, 24 bp, Artificial Sequence
  Synthetic polynucleotide
atggattacaaggatgacgatgac

SEQ ID NO: 42 - DNA, 21 bp, Artificial Sequence
  Synthetic polynucleotide
gcgcatgaactccttgatgac

SEQ ID NO: 43 - protein, 889 aa, Artificial Sequence
  Synthetic polypeptide
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 44 - protein, 602 aa, Artificial Sequence
  Synthetic polypeptide
MIKIATRKYLGKQNVYDIGVERDHNFALKNGFIASNCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 45 - protein, 700 aa, Artificial Sequence
  Synthetic polypeptide
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 46 - protein, 792 aa, Artificial Sequence
  Synthetic polypeptide
MIKIATRKYLGKQNVYDIGVERDHNFALKNGFIASNCAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 47 - protein, 734 aa, Artificial Sequence
  Synthetic polypeptide
MNCEREQLRGNQEAAAAPDTMAQPYASAQFAPPQNGIPAEYTAPHPHPAPEYTGQTTVPEHTLNLYPPAQTHSEQSPADTSAQTVSGTATQTDDAAPTDGQPQTQPSENTENKSQPKGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 48 - protein, 758 aa, Artificial Sequence
  Synthetic polypeptide
MIKIATRKYLGKQNVYDIGVERDHNFALKNGFIASNCFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPANATARVMTNKKTVNPYTNGWKLNPVVGAVYSPEFYAGTVLLCQANQEGSSMYSAPSSLVYTSAMPGFPYPAATAAAAYRGAHLRGRGRTVYNTFRAAAPPPPIPAYGGVVYQDGFYGADIYGGYAAYRYAQPTPATAAAYSDSYGRVYAADPYHHALAPAPTYGVGAMNAFAPLTDAKTRSHADDVGLVLSSLQASIYRGGYNRFAPY

SEQ ID NO: 49 - protein, 1261 aa, Artificial Sequence
  Synthetic polypeptide
MPKFYCDYCDTYLTHDSPSVRKTHCSGRKHKENVKDYYQKWMEEQAQSLIDKTTAAFQQGKIPPTPFSAPPPAGAMIPPPPSLPGPPRPGMMPAPHMGGPPMMPMMGPPPPGMMPVGPAPGMRPPMGGHMPMMPGPPMMRPPARPMMVPTRPGMTRPDRNVIDGGGGSDPKKKRKVDPKKKRKVDPKKKRKVGSTGSRNDGGGGSGGGGSGGGGSGRASPKKKRKVEASIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLAHWVVANNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFANKAVALEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNSGSGPKKKRKVAAAYPYDVPDYAGGRGGGGSGGGGSGGGGSGPAMDYKDHDGDYKDHDIDYKDDDDK

SEQ ID NO: 50 - protein, 1435 aa, Artificial Sequence
  Synthetic polypeptide
MDYKDHDGDYKDHDIDYKDDDDKIDGGGGSDPKKKRKVDPKKKRKVDPKKKRKVGSTGSRNDGGGGSGGGGSGGGGSGRAAAFKPNPINYILGLAIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIAAALPFSRTWDDSFNNKVLVLGSEAQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVRGSTSGSPKKKRKVGGGRGGGGSGGGGSGGGGSGPAMLLQPAPCAPSAGFPRPLAAPGAMHGSQKDTTFTKIFVGGLPYHTTDASLRKYFEGFGDIEEAVVITDRQTGKSRGYGFVTMADRAAAERACKDPNPIIDGRKANVNLAYLGAKPRSLQTGFAIGVQQLHPTLIQRTYGLTPHYIYPPAIVQPSVVIPAAPVPSLSSPYIEYTPASPAYAQYPPATYDQYPYAASPATAASFVGYSYPAAVPQALSAAAPAGTTFVQYQAPQLQPDRMQ

SEQ ID NO: 51 - DNA, 153 bp, Artificial Sequence
  Synthetic polynucleotide
gatatcgcctggatcctgagccaggttgtagctccctttctcatttcggaaacgaaatgagaaccgttgctacaataaggccgtctgaaaagatgtgccgcaacgctctgccccttaaagcttctgctttaaggggcatcgtttaattttttt

SEQ ID NO: 52 - DNA, 154 bp, Artificial Sequence
  Synthetic polynucleotide
gttacaaaagtaagattcactttcagttgtagctccctttctcatttcggaaacgaaatgagaaccgttgctacaataaggccgtctgaaaagatgtgccgcaacgctctgccccttaaagcttctgctttaaggggcatcgtttaattttttt

SEQ ID NO: 53 - DNA, 153 bp, Artificial Sequence
  Synthetic polynucleotide
gagaattctagtagggatgtagatgttgtagctccctttctcatttcggaaacgaaatgagaaccgttgctacaataaggccgtctgaaaagatgtgccgcaacgctctgccccttaaagcttctgctttaaggggcatcgtttaattttttt

SEQ ID NO: 54 - DNA, 153 bp, Artificial Sequence
  Synthetic polynucleotide
gtttcttccacacaaccaaccagtgttgtagctccctttctcatttcggaaacgaaatgagaaccgttgctacaataaggccgtctgaaaagatgtgccgcaacgctctgccccttaaagcttctgctttaaggggcatcgtttaattttttt

SEQ ID NO: 55 - DNA, 19 bp, Artificial Sequence
  Synthetic polynucleotide
ataattcccccaccacctc

SEQ ID NO: 56 - DNA, 35 bp, Artificial Sequence
  Synthetic polynucleotide
cttctttttgattttgtctaaaacccatataatag

SEQ ID NO: 57 - DNA, 19 bp, Artificial Sequence
  Synthetic polynucleotide
ataattcccccaccacctc

SEQ ID NO: 58 - DNA, 27 bp, Artificial Sequence
  Synthetic polynucleotide
ctctatgccagcatttccatataatag

(19) United States
(12) Patent Application Publication
(10) Pub. No.: US 2021/0388351 A1
(43) Pub. Date: Dec. 16, 2021

(54) ARTIFICIAL RNA-GUIDED SPLICING FACTORS

(71) Applicant: The Jackson Laboratory, Bar Harbor, ME (US)

(72) Inventors: Albert Cheng, Bar Harbor, ME (US); Nathaniel Jillette, Bar Harbor, ME (US)

(73) Assignee: The Jackson Laboratory, Bar Harbor, ME (US)

(21) Appl. No.: 17/279,667

(22) PCT Filed: Sep. 27, 2019
(86) PCT No.: PCT/US2019/053482

§ 371 Date: Mar. 25, 2021

Related U.S. Application Data
(60) Provisional application No. 62/738,838, filed on Sep. 28, 2018.

(51) Int. Cl. C12N 15/11; C12N 15/86; C12N 9/22; C12P 19/34

(52) U.S. Cl. CPC C12N 15/111; C12P 19/34; C12N 9/22; C12N 15/86

(57) ABSTRACT

Provided herein, in some aspects, are compositions and methods for artificially modulating alternative splicing, for example, inducing exon inclusion and/or exon exclusion events. In some embodiments, a catalytically inactive programmable nuclease, such as dCasRx, is fused to an RNA-binding protein (or fragment or isoform thereof) and, when guided to a target of interest by a specific guide RNA (gRNA), can regulate alternative splicing in eukaryotic cells.

### RELATED APPLICATION

This application claims the benefit under 35 U.S.C. § 119(e) of U.S. provisional application No. 62/738,838, filed Sep. 28, 2018, which is incorporated by reference herein in its entirety.

### BACKGROUND

RNA, located at the center of the central dogma of molecular biology, regulates diverse biological processes and is itself subject to multiple layers of regulation effected by intricate networks of regulators^{1, 2}. Dysregulation of RNA processes underlies a plethora of diseases³. Tethering of RNA effector domains from natural RNA processing enzymes by heterologous RNA binding proteins (e.g., Pumilio and MS2)^{4, 5}, have allowed artificial regulation of RNA processes, and may enable targeted RNA therapeutics. These artificial RNA effectors require either protein engineering or insertion of artificial tags to target RNA, and depend on short recognition sequences, thus affording only limited targeting flexibility or specificity.

### SUMMARY

Provided herein, in some aspects, are compositions and methods for artificially regulating alternative splicing of mRNA, for example, by inducing exon inclusion and exclusion events. In some embodiments, a catalytically inactive programmable nuclease, such as dCasRx, is fused to an RNA-binding protein (or fragment or isoform thereof) and, when guided to a target of interest by a specific guide RNA (gRNA), can regulate alternative splicing in eukaryotic cells. This versatile, artificial RNA-guided splicing factor can be used, as demonstrated herein, to induce exon inclusion and/or exclusion events at precise locations within a target gene or other genomic locus of interest.

The discovery of RNA-guided RNA nucleases from bacterial CRISPR systems and their adaptation to mammalian cells have enabled programmable RNA degradation as well as RNA-guided regulation of endogenous RNAs (e.g., mRNAs). CasRx is a type IV-D CRISPR-Cas ribonuclease isolated from Ruminococcus flavefaciens XPD3002 with robust activity in degrading target RNAs matching designed gRNA sequences⁸. The data provided herein demonstrates that programmable nucleases (e.g., dCasRx with a mutated nuclease domain (R239A/H244A/R858A/H863A)⁸) can be guided by gRNAs to bind splicing elements to induce exon exclusion and/or inclusion events.

Thus, provided herein, in some aspects, are artificial RNA-guided splicing factors comprising an RNA splicing factor (e.g., RBFOX1 or RBM38) linked to a catalytically inactive programmable nuclease (e.g., dCasRx). In some embodiments, the artificial RNA-guided splicing factor is complexed with a gRNA.

In other aspects, provided herein are compositions comprising a splicing factor (e.g., RBFox1 or RBM38) modified to replace the RNA-binding domain with a first binding partner molecule, a gRNA modified to include a second binding partner molecule that is capable of binding to (e.g., binds to) the first binding partner molecule, and a catalytically inactive programmable nuclease (e.g., dCasRx).

Further provided herein are methods and compositions for modulating RNA splicing. In some embodiments, the methods comprise contacting a cell comprising a gene of interest with the artificial RNA-guided splicing factor of the present disclosure and a gRNA that targets RNA encoded by the gene of interest, and inducing an exon inclusion and/or exclusion event in RNA encoded by the gene of interest.

Also provided herein are methods and compositions for inducing an exon inclusion event. In some embodiments, the methods comprise contacting a cell that expresses a gene of interest with the artificial RNA-guided splicing factor of the present disclosure and a gRNA that targets an intron adjacent to an exon of interest within RNA encoded by the gene of interest, and inducing inclusion of the exon in the RNA encoded by the gene of interest. In other embodiments, the methods comprise a contacting a cell that expresses a gene of interest with (a) a first interaction domain fused to a catalytically inactive programmable nuclease, (b) a second interaction domain fused to a splicing factor, and (c) a gRNA, wherein the first interaction domain and the second interaction domain bind to an inducer agent, and wherein the gRNA targets RNA encoded by a gene of interest; and inducing an exon inclusion and/or exon exclusion event in the RNA encoded by the gene of interest.

The present disclosure also provides, in some aspects, nucleic acids encoding artificial RNA-guided splicing factors.

The present disclosure further provides nucleic acids encoding an RNA splicing factor linked to an N-terminal fragment of a catalytically inactive programmable nuclease linked to an N-terminal fragment of an intein and/or an RNA splicing factor linked to a C-terminal fragment of a catalytically inactive programmable nuclease linked to a C-terminal fragment of an intein.

Also provided herein, in some aspects, are recombinant viral genomes (e.g., AAV genome) comprising the nucleic acids described herein. Further provided herein are viral particles comprising the recombinant viral genomes.

### BRIEF DESCRIPTION OF THE DRAWINGS

FIGS. 1A-1C. Activation of SMN2-E7 by RBFOX1N-dCasRx-C. (FIG. 1A) Schematic of the artificial splicing factor RBFOX1N-dCasRx-C and SMN2 minigene. The RNA binding domain of RBFOX1 was substituted by dCasRx to create an RNA-guided artificial splicing factor RBFOX1N-dCasRx-C that can be guided by guide RNAs (gRNA) to localize RBFOX1 splicing activity to a desired target. The SMN2 minigene on plasmid pCI-SMN2 contains exons 6 (E6) and 8 (E8), which are constitutively spliced, exon 7 (E7), which is alternatively spliced, and the intervening introns, driven by the CMV promoter (pCMV). Two designed target sites for the RBFOX1N-dCasRx-C are indicated by numbered boxes 1 through 4 within the intron between E7 and E8. pCI-F and pCI-R indicate primers used for semi-quantitative RT-PCR assays. (FIG. 1B) Gel image of semi-quantitative splicing RT-PCR using primers pCI-F and pCI-R on SMN2 minigene transcripts in cells co-transfected with control GFP plasmid (pmaxGFP), unfused dCasRx, or RBFOX1N-dCasRx-C, and the indicated guide RNAs (gRNAs). gRNA numbers correspond to those in FIG. 1A with dash indicating the range of gRNAs used. “C” indicates a control gRNA without matching SMN2 minigene sequence. Upper band and the lower band correspond to the exon 7-included and -excluded transcripts, respectively. (FIG. 1C) Column plots showing inc/exc ratio fold changes from quantitative RT-PCR (qRT-PCR) using primer pairs recognizing SMN2 E7-inclusion or exclusion isoforms.

FIGS. 2A-2B. Activation of SMN2-E7 by RBM38-dCasRx and dCasRx-RBM38. (FIG. 2A) Schematic of the artificial splicing factors RBM38-dCasRx, dCasRx-RBM38 and SMN2 minigene. The RNA splicing factor RBM38 was fused N- or C-terminally to dCasRx, to create artificial splicing factors RBM38-dCasRx and dCasRx-RBM38, respectively. The artificial splicing factors were guided to target site 2 by gRNAs with complementary sequence. pCI-F and pCI-R indicate primers used for semi-quantitative RT-PCR assays. (FIG. 2B) Gel image of semi-quantitative splicing RT-PCR using primers pCI-F and pCI-R on SMN2 minigene transcripts in cells co-transfected with RBM38-dCasRx or dCasRx-RBM38, and the indicated gRNAs. “C” indicates a control gRNA without matching SMN2 minigene sequence. Upper band and the lower band correspond to the exon 7-included and -excluded transcripts, respectively.

FIGS. 3A-3B. Activation and repression of SMN2-E7 by differential positioning of RBFOX1N-dCasRx-C, RBM38-dCasRx or dCasRx-RBM38 targeting. (FIG. 3A) Schematic of the artificial splicing factors RBFOX1N-dCasRx-C, RBM38-dCasRx, dCasRx-RBM38 and SMN2 minigene. Sets of three target sites (DN) target downstream of E7 and one target site (EX) targets within E7. (FIG. 3B) Gel image of semi-quantitative splicing RT-PCR using primers pCI-F and pCI-R on SMN2 minigene transcripts in cells co-transfected with dCasRx, RBFOX1N-dCasRx-C, RBM38-dCasRx or dCasRx-RBM38, and the indicated gRNAs. “C” indicates a control gRNA without matching SMN2 minigene sequence; “DN” indicates a pool of three gRNAs targeting downstream of E7; “EX” indicates a gRNA targeting within E7. Upper band and the lower band correspond to the exon 7-included and -excluded transcripts, respectively.

FIGS. 4A-4B. Simultaneous activation and repression of two independent exons by RBFOX1N-dCasRx-C. (FIG. 4A) Schematic of the artificial splicing factor RBFOX1N-dCasRx-C, RBM38-dCasRx and the RG6 as well as SMN2 minigenes. The RG6 contains artificial upstream exon (UX: Upstream eXon), chicken TnT (cTnT) intron 4, an artificial cassette exon (CX: Cassette eXon), cTnT intron 5, and 35nt of cTnT exon 6 (DX: Downstream eXon), driven by CMV promoter (pCMV) [doi:10.1093/nar/gk1967]. A gRNA (RG-SA) was designed to target splice acceptor site of CX. Primer pairs RG6-F and RG6-R can be used to detect isoforms of RG6 transcripts by RT-PCR. A pool of gRNA (DN) target downstream of E7. Primer pairs pCI-F and pCI-R detect isoforms of SMN2. (FIG. 4B) Gel image of semi-quantitative splicing RT-PCR of RG6 and SMN2 minigene transcripts in cells co-transfected with the two minigene plasmids, RBFOX1N-dCasRx-C and the indicated gRNAs. Upper bands and the lower bands for the indicated transcripts correspond to the respective inclusion and exclusion isoforms.

FIGS. 5A-5B. Activation of SMN2-E7 by a three-component two-peptide artificial splicing factor dCasRx/RBFOX1N-MCP-C. (FIG. 5A) Schematic of the artificial splicing factor dCasRx/RBFOX1N-MCP-C and SMN2 minigene. The effector component (RBFOX1N-MCP-C), formed by replacing RNA binding domain of RBFOX1 with MS2 coat protein (MCP) is encoded as a separate peptide from the dCasRx protein but are bridged by a modified gRNA. The modified gRNA was extended on the 3′ end with one or more MS2 hairpins, that can recruit RBFOX1N-MCP-C to the dCasRx ribonucleoprotein complex. The artificial splicing factor was guided to target site 2 by guide RNAs (gRNAs) with complementary sequence. pCI-F and pCI-R indicate primers used for semi-quantitative RT-PCR assays. (FIG. 5B) Gel image of semi-quantitative splicing RT-PCR using primers pCI-F and pCI-R on SMN2 minigene transcripts in cells co-transfected with dCasRx, RBFOX1N-MCP-C, and the indicated gRNAs. “C” indicates a control gRNA without matching SMN2 minigene sequence. 1×MS2 and 5×MS2 indicate gRNA targeting site 2 within the SMN2 intron with one or five MS2 hairpins appended 3′, respectively. Upper band and the lower band correspond to the exon 7-included and -excluded transcripts, respectively.

FIGS. 6A-6B. Simultaneous activation and repression of two independent exons by RBFOX1N-dCasRx-C directed by a polycistronic pre-gRNA. (FIG. 6A) Schematic of the artificial splicing factor RBFOX1N-dCasRx-C, various gRNA architectures, as well as the RG6 and SMN2 minigenes. SMN2-DN gRNAs is a pool of three gRNAs, each expressed by a separate plasmid, targeting the corresponding numbered locations on the SMN2 minigene. RG6-SA targets splice acceptor of RG6 cassette exon (CX). DR-SMN2-2-DR is SMN2 target 2 gRNA flanked by two direct repeats (DR). DR-RG6-SA-DR contains spacer against RG6-CX splice acceptor flanked by two DRs. SMN2-DN-RG6-SA is a polycistronic pre-gRNA with spacers targeting three DN sites on SMN2 downstream intron and RG6-CX splice acceptors intervened by DRs. (FIG. 6B) Gel image of semi-quantitative splicing RT-PCR of RG6 and SMN2 minigene transcripts in cells co-transfected with the two minigene plasmids, RBFOX1N-dCasRx-C and the indicated gRNAs. Upper bands and the lower bands for the indicated transcripts correspond to the respective inclusion and exclusion isoforms.

FIGS. 7A-7B. Exon inclusion induced by dCasRx-DAZAP1(191-407). (FIG. 7A) Schematic of the CRISPR artificial splicing factor dCasRx-DAZAP1(191-407) and SMN2 minigene. Catalytic domain of splicing factor DAZAP1 amino acids 191-407 was fused to the C-terminus of dCasRx, to create CRISPR artificial splicing factor dCasRx-DAZAP1(191-407). To affect splicing, dCasRx-DAZAP1(191-407) was guided to target sites 1, 2 and 3 by gRNAs with complementary sequences. pCI-F and pCI-R indicate primers used for semi-quantitative RT-PCR assays. (FIG. 7B) Gel image of semi-quantitative splicing RT-PCR using primers pCI-F and pCI-R on SMN2 minigene transcripts in cells co-transfected with dCasRx-DAZAP1(191-407), and the indicated gRNAs. “C” indicates a control gRNA without matching SMN2 minigene sequence. Upper band and the lower band correspond to the exon 7-included and -excluded transcripts, respectively.

FIGS. 8A-8B. Exon exclusion induced by binding of dCasRx-tethered U2 auxiliary factor (U2AF) subunits to downstream intron. (FIG. 8A) Schematic of CRISPR artificial splicing factors (CASFx) U2AF65-dCasRx, U2AF35-dCasRx, dCasRx-U2AF65, dCasRx-U2AF35 and SMN2 minigene. To affect splicing, these CASFx were guided to target sites 1, 2 and 3 by gRNAs with complementary sequences. (FIG. 8B) Gel image of semi-quantitative splicing RT-PCR using primers pCI-F and pCI-R on SMN2 minigene transcripts in cells co-transfected with U2AF CASFx, and the indicated gRNAs. “C” indicates a control gRNA without matching SMN2 minigene sequence. Upper band and the lower band correspond to the exon 7-included and -excluded transcripts, respectively.

FIGS. 9A-9B. Exon inclusion induced by binding of dCasRx-U2AF35 to upstream intron. (FIG. 9A) Schematic of the CRISPR artificial splicing factor dCasRx-U2AF35 and SMN2 minigene. To affect splicing, dCasRx-U2AF35 was guided to target sites 1, 2 and 3 downstream of SMN2-E7 or to UP1 target site within the upstream intron. (FIG. 9B) Gel image of semi-quantitative splicing RT-PCR using primers pCI-F and pCI-R on SMN2 minigene transcripts in cells co-transfected with dCasRx-U2AF35, and the indicated gRNAs. “C” indicates a control gRNA without matching SMN2 minigene sequence. Upper band and the lower band correspond to the exon 7-included and -excluded transcripts, respectively.

FIGS. 10A-10B. Chemical-inducible exon activation by three-component two-peptide iCASFx (FIG. 10A) Schematic of the two-peptide artificial splicing factors inducible by rapamycin. The RNA binding module (FKBP-dCasRx or dCasRx-FKBP) and effector module (RBFOX1N-FRB-C, RBM38-FRB, or FRB-RBM38) containing the splicing activator domain are expressed separately as two peptides, fused to FKBP or FRB, respectively. FKBP and FRB can be induced to interact by rapamycin, bringing together the RNA binding module and the splicing activator module, and when guided by gRNAs, assemble at the target to activate exon inclusion.

(FIG. 10B) Gel image of semi-quantitative RT-PCR using primers pCI-F and pCI-R on SMN2 minigene transcripts in cells co-transfected with the indicated constructs, and cultured (“+”) or without (“−”) rapamycin. Upper band and the lower band correspond to the exon 7-included and − excluded transcripts, respectively.

FIGS. 11A-11C. SMN2-E7 induction by RBFOX1N-dCasRx-C in GM03813 SMA Type2 patient fibroblast cells. (FIG. 11A) Plasmids carrying RBFOX1N-dCasRx-C and gRNA targeting a downstream intron were transiently transfected into GM03813 patient fibroblast cells. The splicing of endogenous SMN2 was detected by both (FIG. 11B) semi-quantitative RT-PCR (upper gel image) as well as (FIG. 11C) quantitative RT-PCR (qRT-PCR, lower column plot).

FIGS. 12A-12B. Split CASFx (RBFOX1N-dCasRx-C) architecture. (FIG. 12A) To reduce the size of CASFx to fit the limited payload of AAV vectors, we split CASFx (RBFOX1N-dCasRx-C) within the CasRx coding sequence using NpuDnaE intein trans-splicing elements. The N-split fragment was cloned into an AAV vector creating AAV-CAG-CASFx-N, The C-split CASFx fragment and the gRNA targeting SMN2 (SMN2-DN) were cloned into a separate AAV vector creating AAV-CAG-CASFx-C. These two vectors were co-transfected into HEK293T cells with pCI-SMN2 minigene. Inside cells, the split CASFx reconstituted into full-length CASFx through intein-mediated protein transplicing. (FIG. 12B) Gel image showing splicing induction of SMN2-E7 in samples transfected with three split designs with their split positions indicated.

FIGS. 13A-13B. Exon inclusion induced by binding of SNRPC-dCasRx to downstream intron. (FIG. 13A) Schematic of the CRISPR artificial splicing factor SNRPC-dCasRx and SMN2 minigene. To affect splicing, SNRPC-dCasRx was guided to target sites 1, 2 and 3 downstream of SMN2-E7 within the downstream intron. (FIG. 13B) Gel image of semi-quantitative splicing RT-PCR using primers pCI-F and pCI-R on SMN2 minigene transcripts in cells co-transfected with SNRPC-dCasRx, and the indicated gRNAs. “C” indicates a control gRNA without matching SMN2 minigene sequence. Upper band and the lower band correspond to the exon 7-included and -excluded transcripts, respectively.

FIGS. 14A-14B. Exon inclusion induced by binding of dNMCas9-RBM38 to downstream intron. (FIG. 14A) Schematic of the CRISPR artificial splicing factor dNMCas9-RBM38 and SMN2 minigene. To affect splicing, dNMCas9-RBM38 was guided to target sites 1, 2 or 3 downstream of SMN2-E7 within the downstream intron. (FIG. 14B) Gel image of semi-quantitative splicing RT-PCR using primers pCI-F and pCI-R on SMN2 minigene transcripts in cells co-transfected with dNMCas9-RBM38, and the indicated gRNAs. “C” indicates a control gRNA without matching SMN2 minigene sequence. Upper band and the lower band correspond to the exon 7-included and -excluded transcripts, respectively.

### DETAILED DESCRIPTION

The present disclosure provides methods and compositions for modulating RNA splicing. In eukaryotes and some prokaryotes, transcribed RNA comprises exons, which encode proteins, and intervening intron sequences, which do not encode proteins. Splicing is the process of removing the intron sequences and joining the remaining exon sequences to produce a mature messenger RNA (mRNA).

Alternative splicing occurs when a single gene codes for multiple proteins because one or more exons are included or excluded from the mature mRNA. The production of alternatively spliced mRNAs is regulated by trans-activating proteins (splicing factors) that bind to cis-activating sites on the mRNA transcript (splice acceptor sites). The proteins translated from alternatively spliced mRNAs have different amino acid sequences, which often translate into differences in biological function.

### Splicing Factors

Splicing is the process of removing introns from a pre-mRNA molecule and joining the remaining exons in a mRNA molecule. Some aspects of the present disclosure provide artificial RNA-guided splicing factors that comprise an RNA splicing factor. An RNA splicing factor is a protein involved in the removal of introns, and in some instances, exons, from transcribed pre-messenger RNA (pre-mRNA). The resulting processed mRNA includes mostly exons, which are nucleotide sequences within a gene that encode part of the processed mRNA, as opposed to introns, which are nucleotide sequences within a gene that are removed by mRNA splicing.

An RNA splicing factor comprises an RNA-binding domain and a splicing domain. An RNA-binding domain (also referred to in the art as an RNA recognition motif) binds to RNA (e.g., single-stranded RNA or a secondary structure). A splicing domain of an RNA splicing factor is a catalytic domain. Binding of the splicing factor to RNA through the RNA-binding domain enables exertion of its function as a splicing factor. In some embodiments, as discussed elsewhere herein, an RNA-binding domain of a splicing factor is replaced with a catalytically inactive RNA-guided programmable nuclease. In some embodiments, an RNA splicing factor comprises a functional fragment (e.g., catalytic domain) of a splicing factor. In other embodiments, the RNA splicing factor comprises both the binding domain and the splicing domain (or functional fragments thereof). In yet other embodiments, the RNA splicing factor comprises a full-length functional splicing factor, which includes the entire amino acid sequence encoded by the splicing factor gene. It should be understood that an RNA splicing factor as used herein, when isolated as a fragment of a full length splicing factor, retains its function/activity (e.g., RNA-binding and/or splicing).

Non-limiting examples of splicing factors that may be used as provided herein include 9G8, CUG-BP1, DAZAP1, ESRP1, ESRP2, ETR-3, FMRP, Fox-1, Fox-2, hnRNP A0, hnRNP A1, hnRNP A2/B1, hnRNP A3, hnRNP C, hnRNP C1, hnRNP C2, hnRNP D, hnRNP D0, hnRNP DL, hnRNP E1, hnRNP E2, hnRNP F, hnRNP G, hnRNP H1, hnRNP H2, hnRNP H3, hnRNP I (PTB), hnRNP J, hnRNP K, hnRNP L, hnRNP LL, hnRNP M, hnRNP P (TLS), hnRNP Q, hnRNP U, HTra2α, HTra2β1, HuB, HuC, HuD, HuR, KSRP, MBNL1, Nova-1, Nova-2, nPTB, PSF, QKI, RBM25, RBM4, RBM5, Sam68, SAP155, SC35, SF1, SF2/ASF, SLM-1, SLM-2, SRm160, SRp20, SRp30c, SRp38, SRp40, SRp54, SRp55, SRp75, TDP43, TIA-1, TIAL1, YB-1, and ZRANB2 (see, e.g., Giulietti M et al. Nucleic Acids Res 2013; 41:D125-131). In some embodiments, the splicing factor is selected from RBFOX1, RBM38, DAZAP1, U2AF65, U2AF35, HNRNPH1, TRA2A, TRA2B, SYMPK, CPSF2, SRSF1, 9G8, PTB1/2, MBNL1/2/3, ESRP1, NOVA1, NOVA2, CELF4, SRM160, and SNRPC (U1C). In some embodiments, the splicing factor is selected from RBFOX1 and RBM38.

The RNA binding fox-1 homolog 1 (RBFOX1) gene (Gene ID: 54715) encodes the RBFOX1 protein (also known as FOX1 or A2BP1), which regulates alternative splicing of a variety of RNA transcripts that are critical for neuronal function. Abnormalities in RBFOX1 that cause aberrant RBFOX1 activity are associated with autism and other neurodevelopmental and neuropsychiatric disorders, including intellectual disability, epilepsy, attention deficit hyperactivity disorder, schizophrenia, and Alzheimer disease. In some embodiments, an RNA splicing factor comprises RBFOX1. In some embodiments, an RNA splicing factor of the present disclosure comprises a catalytic domain of RBFOX1.

The RNA binding motif protein 38 (RBM38) gene (Gene ID: 55544) encodes the RBM38 protein, which regulates alternative splicing during late erythroid differentiation, where it regulates the translation of p53 and PTEN tumors. Loss of RBM38 enhances p53 expression and decreases PTEN expression, thereby promoting lymphomagenesis. In some embodiments, an RNA splicing factor comprises RBM38. In some embodiments, an RNA splicing factor of the present disclosure comprises a catalytic domain of RBM38.

The DAZ associated protein 1 (DAZAP1) gene (Gene ID: 26528) encodes the DAZAP1 RNA-binding protein, which is involved in mammalian development and spermatogenesis. DAZAP1 promotes inclusion of weak exons and neutralizes splicing inhibitors when recruited to RNA. In some embodiments, an RNA splicing factor comprises DAZAP1. In some embodiments, an RNA splicing factor of the present disclosure comprises a catalytic domain of DAZAP1.

U2AF65 (Gene ID: 11338), together with U2AF35 (Gene ID: 7307), forms the U2 small nuclear ribonucleoprotein auxiliary factor (U2AF) complex, a component of splicing machinery. The large subunit (U2AF65) of the complex binds to the polypyrimidine tract of introns early in spliceosome assembly and also includes a protein-protein interaction domain that binds and recruits other splicing factors. The small subunit (U2AF35) is required for constitutive RNA splicing and also functions as a mediator of enhancer-dependent splicing, where it binds to an enhancer and acts as a bridge to recruit U2AF65 to an adjacent intron. In some embodiments, an RNA splicing factor comprises U2AF65. In some embodiments, an RNA splicing factor comprises U2AF35. In some embodiments, an RNA splicing factor of the present disclosure comprises a catalytic domain of U2AF35.

The heterogeneous nuclear ribonucleoprotein H1 (HNRNPH1) gene (Gene ID: 3187) encodes a member of a subfamily of ubiquitously expressed heterogeneous nuclear ribonucleoproteins (hnRNPS) including additional family members HNRNPA1 and PTBP1. HnRNPs are a family of RNA binding protein that bind heterogeneous nuclear RNA and are associated with pre-mRNA processing and other aspects of mRNA metabolism and transport. In some embodiments, an RNA splicing factor comprises HNRNPH1. In some embodiments, an RNA splicing factor of the present disclosure comprises a catalytic domain of HNRNPH1.

The transformer 2 alpha homolog (TRA2A) gene (Gene ID: 29896) encodes the TRA2A protein. TRA2A is a sequence-specific RNA-binding protein that participates in the control of pre-mRNA splicing. In some embodiments, an RNA splicing factor comprises TRA2A. In some embodiments, an RNA splicing factor of the present disclosure comprises a catalytic domain of TRA2A.

The transformer 2 beta homolog (TRA2B) gene (Gene ID: 6434) encodes the TRA2B protein. TRA2B is a splicing regulator that plays a role in pre-mRNA processing, splicing patterns, and gene expression. It is involved in spermatogenesis and neurologic disease through regulation of nuclear autoantigenic sperm protein (NASP), microtubule associated protein tau (MAPT), and survival motor neurons (SMN) genes. In some embodiments, an RNA splicing factor comprises TRA2B. In some embodiments, an RNA splicing factor of the present disclosure comprises a catalytic domain of TRA2B.

The symplekin (SYMPK) gene (Gene ID: 8189) encodes the SYMPK protein. SYMPK regulates polyadenylation and promotes gene expression as part of a polyadenylation protein complex. The SYMPK protein is thought to serves as a scaffold for recruiting other members of the polyadenylation complex. In some embodiments, an RNA splicing factor comprises SYMPK. In some embodiments, an RNA splicing factor of the present disclosure comprises a catalytic domain of SYMPK.

The cleavage and polyadenylation specific factor 2 (CPSF2) gene (Gene ID: 53981) encodes the CPSF2 protein, a component of the CPSF complex. The CPSF complex regulates pre-mRNA 3-end formation and processing by recognizing the AAUAAA signal sequence and recruiting other factors that promote cleavage and polyadenylation. In some embodiments, an RNA splicing factor comprises CPSF2. In some embodiments, an RNA splicing factor of the present disclosure comprises a catalytic domain of CPSF2.

The serine and arginine rich splicing factor 1 (SRSF1) gene (Gene ID: 6426) encodes the SRSF1 protein, which activates or represses splicing depending on its phosphorylation state and its interaction partners. SRSF1 promotes spliceosome assembly, constitutive pre-mRNA splicing, and regulates alternative splicing. In some embodiments, an RNA splicing factor comprises SRSF1. In some embodiments, an RNA splicing factor of the present disclosure comprises a catalytic domain of SRSF1.

The serine and arginine rich splicing factor 7 (SRSF7) gene (Gene ID: 6432) encodes the SRSF7 (9G8) protein. The 9G8 protein promotes spliceosome assembly and constitutive pre-mRNA splicing and regulates mRNA export from the nucleus. In some embodiments, an RNA splicing factor comprises 9G8. In some embodiments, an RNA splicing factor of the present disclosure comprises a catalytic domain of 9G8.

The polypyrimidine tract binding protein 1 (PTBP1) gene (Gene ID: 5725) encodes the PTB1 protein. The PTB1 protein is a negative regulator of alternative splicing, causing exon-skipping in numerous pre-mRNAs. PTB1 also regulators 3′-end processing of mRNA and mRNA stability. In some embodiments, an RNA splicing factor comprises PTB1. In some embodiments, an RNA splicing factor of the present disclosure comprises a catalytic domain of PTB1.

The polypyrimidine tract binding protein 2 (PTBP2) gene (Gene ID: 58155) encodes the PTB2 protein. The PTB2 protein regulates pre-mRNA splicing in neurons and germ cells. PTB2 also regulates 3′-end processing of mRNA and mRNA stability. In some embodiments, an RNA splicing factor comprises PTB2. In some embodiments, an RNA splicing factor of the present disclosure comprises a catalytic domain of PTB2.

The muscleblind like splicing regulator 1 (MBNL1) gene (Gene ID: 4154) encodes the MBNL1 protein. The MBNL1 protein is a sequence-specific pre-mRNA splicing factor that binds RNA through pairs of highly conserved zinc fingers. It is predominantly expressed in skeletal muscles, neuronal tissues, thymus, liver, and kidney tissues, and it is important for the terminal differentiation of myocytes and neurons. MBNL1 transcripts are alternatively splicing to generate a variety of protein isoforms, and inclusion of exon 5 is critical for differentiation of hear and muscle. Perturbation of MBNL1 activity is associated with myotonic dystrophy. In some embodiments, an RNA splicing factor comprises MBNL1. In some embodiments, an RNA splicing factor of the present disclosure comprises a catalytic domain of MBNL1.

The muscleblind like splicing regulator 2 (MBNL2) gene (Gene ID: 10150) encodes the MBNL2 protein. The MBNL2 protein is a sequence-specific pre-mRNA splicing factor that binds RNA through pairs of highly conserved zinc fingers. MBNL2 acts as either an activator or repressor of splicing on specific pre-mRNA targets, including cardiac troponin-T, insulin receptor, and CELF proteins. Perturbation of MBNL2 activity is associated with myotonic dystrophy. In some embodiments, an RNA splicing factor comprises MBNL2. In some embodiments, an RNA splicing factor of the present disclosure comprises a catalytic domain of MBNL2.

The muscleblind like splicing regulator 3 (MBNL3) gene (Gene ID: 55796) encodes the MBNL3 protein. The MBNL3 protein is a sequence-specific pre-mRNA splicing factor that binds RNA through a pair of highly-conserved zinc fingers. MBNL3 may function in the regulator of alternative splicing and may play a role in the pathophysiology of myotonic dystrophy. In some embodiments, an RNA splicing factor comprises MBNL3. In some embodiments, an RNA splicing factor of the present disclosure comprises a catalytic domain of MBNL3.

The epithelial splicing regulatory protein 1 (ESRP1) gene (Gene ID: 54845) encodes the ESPR1 splicing regulator protein. The ESPR1 protein is a regulator of alternative splicing in epithelial cells whose expression is down-regulated during the epithelial-mesenchymal transition, a fundamental development process that is abnormally activated in cancer metastasis. ESPR1 is upregulated in numerous cancers, including ovarian and cervical cancers. In some embodiments, an RNA splicing factor comprises ESPR1. In some embodiments, an RNA splicing factor of the present disclosure comprises a catalytic domain of ESPR1.

The epithelial splicing regulator protein 2 (ESPR2) gene (Gene ID: 80004) encodes the ESPR2 splicing regulator protein. The ESPR2 protein is a regulator of alternative splicing in epithelial cells whose expression is down-regulated during the epithelial-mesenchymal transition. ESPR2 is upregulated in numerous cancers, including ovarian and cervical cancers. In some embodiments, an RNA splicing factor comprises ESPR2. In some embodiments, an RNA splicing factor of the present disclosure comprises a catalytic domain of ESPR2.

The NOVA alternative splicing regulator 1 (NOVA1) gene (Gene ID: 4857) encodes the NOVA1 protein. The NOVA1 protein is a neuron-specific RNA-binding protein, a member of paraneoplastic disease antigens that is recognized and inhibited by paraneoplastic antibodies. These antibodies are found in the sera of patients with paraneoplastic opsoclonus-ataxia, breast cancer, and small cell lung cancer. In some embodiments, an RNA splicing factor comprises NOVA1. In some embodiments, an RNA splicing factor of the present disclosure comprises a catalytic domain of NOVA1.

The NOVA alternative splicing regulator 2 (NOVA2) gene (Gene ID: 4858) encodes the NOVA2 protein. The NOVA2 protein is a neuron-specific RNA-binding protein that regulates splicing in a series of RNA molecules that guide axons to the correct location in developing brains. In some embodiments, an RNA splicing factor comprises NOVA2. In some embodiments, an RNA splicing factor of the present disclosure comprises a catalytic domain of NOVA2.

The CUGBP Elav-like family member 4 (CELF4) gene (Gene ID: 56853) encodes the CELF4 protein. The CELF4 protein regulates pre-mRNA alternative splicing and may also be involved in mRNA editing and translation. CELF4 is primarily expressed at axons in neuronal tissue and deficits in CELF4 function are associated with brain disorders such as epilepsy. In some embodiments, an RNA splicing factor comprises CELF4. In some embodiments, an RNA splicing factor of the present disclosure comprises a catalytic domain of CELF4.

The serine and arginine repetitive matrix 1 (SRRM1) gene (Gene ID: 10250) encodes the SRM160 protein. The SRM160 protein contains an RNA recognition motif (RRM) and forms a splicing coactivator heterodimer with the SRM300 protein, a complex that promotes interactions between splicing factors bound to pre-mRNA. In some embodiments, an RNA splicing factor comprises SRM160. In some embodiments, an RNA splicing factor of the present disclosure comprises a catalytic domain of SRM160.

The U1 small nuclear ribonucleoprotein C (SNRPC; aka U1C) gene (Gene ID: 6631) encodes one of the specific protein components of the U1 small nuclear ribonucleoprotein (snRNP) particle required for the formation of the spliceosome. The encoded protein participates in the processing of nuclear precursor messenger RNA splicing. In some embodiments, an RNA splicing factor comprises SNRPC. In some embodiments, an RNA splicing factor of the present disclosure comprises a catalytic domain of SNRPC.

Provided herein, in some embodiments, are methods and compositions for modulating RNA splicing. Modulation of RNA splicing may include inducing an exon inclusion event (whereby a particular exon is included in the processed mRNA) and/or inducing an exon exclusion event (whereby a particular exon is excluded from the processed mRNA).

In some embodiments, the methods comprise contacting a cell comprising a gene of interest with the artificial RNA-guided splicing factor and a guide RNA (gRNA) that targets RNA encoded by the gene of interest, and inducing an exon inclusion event or an exclusion event in RNA encoded by the gene of interest. In some embodiments, the methods comprise inducing an exon inclusion event and an exclusion event in RNA encoded by the gene of interest. An exon inclusion event is a form of alternative splicing in which an exon otherwise excluded from processed mRNA is included (present) in the processed mRNA. An exon exclusion event is a form of alternative splicing in which an exon otherwise included in processed mRNA is excluded from (absent) in the processed mRNA.

In some embodiments, the present disclosure provides methods and compositions for modulating RNA splicing comprising contacting a cell comprising two genes of interest with the artificial RNA-guided splicing factor and two separate (independent) gRNAs or a concatemer of tandem gRNAs, wherein one of the gRNAs (e.g., a first gRNA) targets RNA encoded by one of the genes of interest (e.g., a first gene of interest) and the other of the gRNAs (e.g., a second gRNA) targets RNA encoded by the other gene of interest (e.g., a second gene of interest), and inducing an exon inclusion even in RNA encoded by one of the genes of interest (e.g., the first gene of interest) and inducing an exon exclusion event in RNA encoded by the other gene of interest (e.g., the second gene of interest). As used herein, a concatemer is a long, contiguous nucleic acid molecule that comprises multiple discrete nucleic acid sequences (e.g., each encoding a gRNA) arranged in tandem. In some embodiments, the nucleic acid sequences arranged in tandem encode gRNAs. In some embodiments, the concatemer comprises nucleic acid sequences that encode two gRNAs, three gRNAs, four gRNAs, five gRNAs, six gRNAs, seven gRNAs, eight gRNAs, nine gRNAs, or ten gRNAs.

In some embodiments, the present disclosure provides methods and compositions for inducing an exon inclusion event. In some embodiments, the methods comprise contacting a cell that expresses a gene of interest with the artificial RNA-guided splicing factor and a gRNA that targets an intron adjacent to (e.g., downstream from or upstream from) an exon of interest within RNA encoded by the gene of interest, and inducing inclusion of the exon in the RNA encoded by the gene of interest.

In some embodiments, the present disclosure provides methods and compositions for inducing an exon inclusion event. In some embodiments the methods comprise contacting a cell that expresses a gene of interest with the artificial RNA-guided splicing factor and a gRNA or a concatemer of tandem gRNAs that target(s) an intron adjacent to the exon of interest within RNA encoded by the gene of interest, and inducing inclusion of the exon in the RNA encoded by the gene of interest.

In some embodiments, a method of the present disclosure results in a change in the ratio of inclusion of the exon to exclusion of the exon. In some embodiments, the ratio of inclusion of the exon to exclusion of the exon is increased by at least 1.5 fold, at least 2 fold, at least 5 fold, at least 10 fold, or at least 20 fold relative to a control. In some embodiments, the ratio of inclusion of the exon to exclusion of the exon is increased by at least 1.1 fold, 1.2 fold, 1.3 fold, 1.4 fold, 1.5 fold, 1.6 fold, 1.7 fold, 1.8 fold, or 1.9 fold relative to a control.

In some aspects, the present disclosure provides compositions comprising the artificial RNA-guided splicing factor and a gRNA or a concatemer of tandem gRNAs. In some embodiments, the present disclosure provides compositions comprising an artificial RNA-guided splicing factor. In some embodiments, the compositions further comprise a carrier. As used herein, a carrier refers to an organic or inorganic ingredient, natural or synthetic, with which the active ingredient is combined to facilitate an intended use. Active ingredients (e.g., RNA splicing factor, gRNA or concatemer gRNAs, catalytically inactive programmable nuclease) may be admixed or compounded with any conventional pharmaceutical carrier or excipient.

### Programmable Nucleases

RNA splicing factors of the present disclosure, in some embodiments, are linked to a catalytically inactive programmable nuclease. Programmable nuclease are nucleases that can be targeted to a specific site (e.g., nucleotide or sequence of nucleotides) within a nucleic acid (e.g., within a gene (or genome) and/or a gene transcript). Examples of the most common programmable nucleases include zinc-finger nucleases (ZFNs), transcription activator-like effector nucleases (TALENs) and RNA-guided engineered nucleases (RGENs) derived from the bacterial clustered regularly interspaced short palindromic repeat (CRISPR)-Cas (CRISPR-associated) system. Programmable nucleases include both deoxyribonucleases, which catalyze cleavage of DNA, and ribonucleases, which catalyze cleavage of RNA. Several known programmable nucleases, such as Cas nucleases, have been shown to function as both a deoxyribonuclease and a ribonuclease. In some embodiments, a programmable nuclease of the present disclosure is a programmable deoxyribonuclease. In other embodiments, a programmable nuclease of the present disclosure is a programmable ribonuclease.

Non-limiting examples of programmable nucleases include Cas nucleases, such as type VI-D CRISPR-Cas ribonucleases, Leptotrichia wadei C2c2/Cas13a ribonucleases (see, e.g., Abudayyeh O O et al. Science 2016; 353(6299):aaf5573; and Abudayyeh O O et al. Nature 2017; 550:280-284), Cas13b ribonucleases (see, e.g., Cox D B T et al. Science 2017; 358(6366):1019-1027), Cas13d ribonucleases (see e.g., Zhang et al., Cell 2018 175(1), 212-223 e217 and Neisseria meningitidis Cas9 endonuclease (see, e.g., Lee C M et al. Mol Ther 2016; 24(3):645-654). In some embodiments, the programmable ribonuclease is a type VI-D CRISPR-Cas ribonuclease is dCasRx (Konermann, S et al. Cell 2018; 173:665-676). Other programmable nucleases may be used, in some embodiments, including Staphylococcus aureus Cas9, Streptococcus pyogenes Cas9, Campylobacter jejuni Cas9, and Neisseria meningitides Cas9, each of which have been shown to be capable of targeting both DNA and RNA (see, e.g., Strutt S C et al. eLife 2018; 7:e32724; Dugar et al., Molecular Cell 2018; 69(5), 893-905 e897; and Rousseau B A et al. Molecular Cell 2018; 69(5):P906-914). In some embodiments, the programmable nuclease is selected from catalytically inactive type VI-D CRISPR-Cas ribonucleases, C2c2/Cas13a ribonucleases, Cas13b ribonucleases, and Cas13d ribonucleases. In some embodiments, the programmable nuclease is a Neisseria meningitides Cas9 protein. Programmable nucleases are rendered inactive, in some embodiments, through mutation of the naturally-occurring enzymes.

The dCasRx catalytically inactive programmable ribonuclease is a ribonuclease effector protein derived from the Ruminococcus flavefaciens strain XPD3002. CasRx is a class 2 CRISPR-Cas ribonuclease protein that comprises two HEPN (RxxxxH) ribonuclease motifs. Point mutations (i.e., R295A, H300A, R849A, H854A) of catalytic residues in the HEPN motifs of the CasRx protein results in inactivation of ribonuclease activity without inhibiting the targeting of dCasRx to the coding portion of the mRNA.

In some embodiments, an RNA splicing factor is fused to a catalytically inactive programmable nuclease. A fusion protein comprises a two or more linked polypeptides that are encoded by a single or separate nucleic acid sequences (e.g., two or more separate nucleic acid sequences). Fusion proteins are typically recombinantly produced, wherein the polynucleotides that encode the fusion protein are in a system that supports the expression of the two or more linked polynucleotides, for example, and the translation of the resulting polynucleotides into recombinant polypeptides. Fusion proteins (or other fusion polypeptides) may be configured in multiple arrangements. An RNA splicing factor, in some embodiments, is fused to the amino terminus (N terminus) of a catalytically inactive programmable nuclease. In other embodiments, an RNA splicing factor is fused to the carboxy terminus (C terminus) of a catalytically inactive programmable nuclease.

In some embodiments, the catalytically inactive programmable nuclease is in a “split” form, whereby the coding sequence of the nuclease is split, creating two fragments that can be encoded separately (e.g., encoded on separate nucleic acids and/or vectors) but joined together once expressed to render an active artificial RNA-guided splicing factor. Such a split form allows, e.g., for the packaging of the active artificial RNA-guided splicing factor in two or more vectors, such as viral vectors including AAV. In some embodiments, the two fragments each comprise a fragment of an intein which can be (self-) spliced together. For example, in some embodiments the artificial RNA-guided splicing factor comprises an N-terminal fragment of a catalytically inactive programmable nuclease linked to an N-terminal fragment of an intein and a C-terminal fragment of a catalytically inactive programmable nuclease linked to a C-terminal fragment of an intein, wherein the N-terminal fragment and the C-terminal fragment of the intein catalyze joining of the N-terminal and C-terminal fragments of the catalytically inactive programmable nuclease to produce the full-length artificial RNA-guided splicing factor. In some embodiments the intein utilized is the Npu DnaE intein (see e.g., Zettler et al., FEBS Lett. 2009 Mar. 4; 583(5):909-14). Inteins suitable for use in embodiments described herein are well known in the art, and include those provided in International Publication No. WO 2019/075200, the contents of which are hereby incorporated in their entirety.

### Guide RNA

Compositions of the present disclosure, in some embodiments, comprise an artificial RNA-guided splicing factor and a guide RNA (gRNA). A gRNA is a short RNA (e.g., synthetic RNA) composed of a scaffold sequence used for programmable nuclease (e.g., Cas) binding and a ˜20-25 nucleotide spacer that defines a nucleic acid target. In some embodiments, a spacer is 15 to 30 nucleotides. In some embodiments, the spacer is 15, 16, 17, 18, 19, 29, 21, 22, 23, 24, 25, 26, 27, 28, 29, or 30 nucleotides. In some embodiments, a spacer is 22 nucleotides.

In some embodiments, a composition comprises an artificial RNA-guided splicing factor and a concatemer (two or more, for example, three, four, or five) of tandem (e.g., adjacent) gRNAs (also referred to as a pre-gRNA molecule). In some embodiments, an artificial RNA-guided splicing factor is complexed with (e.g., non-covalently bound to) a gRNA. In some embodiments, a composition comprises a gRNA that targets a first gene of interest. In some embodiments, a composition further comprises an additional RNA (e.g., 1, 2, 3, 4, or more) that targets a second gene of interest.

### Genes of Interest

SMN2 Gene

In some embodiments, a gRNA targets the survival of motor neuron 2 SMN2 gene (Gene ID: 6607), which encodes the survival of motor neuron (SMN) protein. A C840T mutation in Exon 7 of the SMN2 gene creates an exonic splicing suppressor (ESS) that leads to exclusion of Exon 7 during pre-mRNA splicing. The exclusion of Exon 7 results in roughly 90% truncated, non-functional SMN protein, which is rapidly degraded. Subjects with SMN2 exon exclusion have approximately only 10% of functional SMN protein, which is insufficient to sustain survival of spinal motor neurons in the CNS, resulting in spinal muscular atrophy (SMA).

Spinal muscular atrophies (OMIM: 253300, 253550, 253400, and 271150) are a rare, debilitating family of autosomal recessive neuromuscular diseases characterized by motor neuron degeneration and loss of muscle strength. Four types of SMA (I-IV) are recognized depending upon the age of onset, the maximum muscular activity achieved, and survival. In individuals with SMA, degeneration of motor neurons in the spinal cord results in skeletal muscular atrophy and weakness most commonly involving the limbs.

Thus, in some embodiments, provided herein are methods and compositions for treating a subject (e.g., a human subject) having (e.g., diagnosed with) SMA. In some embodiments, the methods comprise administering to the subject an artificial RNA-guided splicing factor as provided herein and a gRNA that targets the SMN2 gene, e.g., an intron adjacent to Exon 7. In some embodiments, the artificial RNA-guided splicing factor and gRNA are formulated in a lipid nanoparticle, such as a cationic lipid nanoparticle.

The SMN1 gene (Gene ID: 6606) is a homolog of SMN2. The sequence difference between SMN1 and SMN2 is a single nucleotide in exon 7 (+6 position), which is a “C” (cytosine) in SMN1 and a “T” (thymine) in SMN2. This thymine creates an exonic splicing silencer (ESS) in SMN2, which results in inefficient splicing and inclusion of Exon 7 (see, e.g., Kashima, T. and Manley, J. L. Nature Genetics, 2003 34(4): 460-463).

In some embodiments, the exon subjected to an exon inclusion event is Exon 7 of SMN2. In some embodiments, Exon 7 comprises a thymine “T” at the +6 position of Exon 7. In some embodiments, Exon 7 comprises a cytosine “C” at the +6 position of Exon 7. In some embodiments, a gRNA targets an intron between Exon 7 and Exon 8 of SMN2. In some embodiments, a gRNA targets an intron between Exon 6 and Exon 7 of SMN2. In some embodiments, a gRNA targets Exon 7. In some embodiments, the gRNA has a sequence as set forth in SEQ ID NOs: 2-6, 8, or 10.

RG6 Minigene

In some embodiments, a gene of interest is a RG6 minigene. In some embodiments, the additional gRNA targets a splice acceptor site of the RG6 minigene (Orengo, J. et al. Nucleic Acids Research 2006; 34(22):e148). The RG6 minigene is a biochromatic alternative splicing reporter for cardiac troponin T upstream of dsRED and EGFP fluorescent reporter proteins. Alternative splicing of a 28 nucleotide cassette exon shifts the reading frame between the dsRED and EGFP reporter proteins.

### Artificial RNA-Guided Splicing Factor Complexes

Also provided herein are artificial RNA-guided splicing factor complexes that modulate RNA splicing. In some embodiments, an artificial RNA-guided splicing factor complex comprises an RNA splicing factor and a catalytically inactive programmable nuclease that are separately recruited to form a complex with (to bind directly or indirectly to) a gRNA targeting a gene of interest (e.g., targeting mRNA encoded by a gene of interest).

Also provided herein, in some aspects, are compositions comprising a splicing factor (e.g., any one of the splicing factors described herein) modified to replace the RNA-binding domain with a first binding partner molecule (e.g., MS2 bacteriophage coat protein), a guide RNA modified to include a second binding partner molecule that binds to the first binding partner molecule (e.g., a stem-loop structure from the MS2 bacteriophage genome), and a catalytically inactive programmable nuclease (e.g., dCasRx). Thus, in some embodiments, a splicing factor comprises a binding partner molecule instead of an RNA-binding domain.

Binding partner molecules may be any two molecules that bind to each other (e.g., transiently or stably). In some embodiments, the binding partner molecules are proteins (e.g., ligand/receptor pairs). In some embodiments, the binding partner molecules are nucleic acids (e.g., complementary nucleic acids). In some embodiments, one binding partner molecule is a protein and the other binding partner molecule is a nucleic acid (e.g., MS2 bacteriophage coat protein and a stem-loop structure from the MS2 bacteriophage genome).

In some embodiments, the first binding partner molecule is a MS2 bacteriophage coat protein (see, e.g., Johansson H E et al. Sem Virol. 1997; 8(3):176-185). In some embodiments, the second binding partner molecule is a stem-loop structure from the MS2 bacteriophage genome. In some embodiments, a modified gRNA comprises at least two (e.g., 2, 3, 4, or 5) copies of the second binding partner molecule.

In some embodiments, the catalytically inactive programmable nuclease is a type VI-D CRISPR-Cas ribonuclease. In some embodiments, the type VI-D CRISPR-Cas ribonuclease is dCasRx. Other catalytically inactive programmable nuclease may be used and are described elsewhere herein.

Further provided herein, in some aspects are methods of modulating RNA splicing, the methods comprising contacting a cell comprising a gene of interest with (a) a splicing factor modified to replace the RNA-binding domain with a first binding partner molecule (e.g., MS2 bacteriophage coat protein), (b) a guide RNA modified to include a second binding partner molecule that is capable of binding to the first binding partner molecule (e.g., a stem-loop structure from the MS2 bacteriophage genome), and (c) a catalytically inactive programmable nuclease (e.g., dCasRx), wherein the gRNA targets RNA encoded by the gene of interest and inducing an exon inclusion and/or exclusion event in the RNA encoded by the gene of interest.

In some embodiments, the methods comprise contacting a cell that expresses a gene of interest with (a) a splicing factor modified to replace the RNA-binding domain with a first binding partner molecule (e.g., MS2 bacteriophage coat protein), (b) a guide RNA (gRNA) modified to include a second binding partner molecule that is capable of binding to the first binding partner molecule (e.g., a stem-loop structure from the MS2 bacteriophage genome), and (c) a catalytically inactive programmable nuclease (e.g., dCasRx), wherein the gRNA targets an intron adjacent to an exon of interest within RNA encoded by the gene of interest, and inducing inclusion of the exon in the RNA encoded by the gene of interest.

In some embodiments, the present disclosure provides methods of modulating RNA splicing comprising contacting a cell comprising a gene of interest with (a) a splicing factor modified to replace the RNA-binding domain with a first binding partner molecule, (b) a guide RNA modified to include a second binding partner molecule that is capable of binding to the first binding partner molecule, and (c) a catalytically inactive programmable nuclease, wherein the guide RNA targets RNA encoded by the gene of interest and, inducing an exon inclusion and/or exclusion event in the RNA encoded by the gene of interest.

In some embodiments, the present disclosure provides methods of inducing an exon inclusion event comprising contacting a cell that expresses a gene of interest with (a) a splicing factor modified to replace the RNA-binding domain with a first binding partner molecule, (b) a guide RNA (gRNA) molecule modified to include a second binding partner that is capable of binding to the first binding partner molecule, and (c) a catalytically inactive programmable nuclease, wherein the gRNA targets an intron adjacent to an exon of interest within RNA encoded by the gene of interest, and inducing inclusion of the exon in the RNA encoded by the gene of interest. In some aspects, the present disclosure provides compositions comprising an artificial RNA-guided splicing factor and a gRNA.

iCASFx

Also provided herein, in some aspects, are methods and compositions for exon inclusion comprising a two-peptide, inducible CRISPR Artificial Splicing Factors (iCASFx) system. In some embodiments, the iCASFx system comprises a first interaction domain fused to a catalytically inactive programmable nuclease, a second interaction domain fused to splicing factor, wherein the first interaction domain and the second interaction domain dimerize in the presence of an inducer agent, and a guide RNA. Interaction domains are molecules (e.g., proteins) that can binds to each other or can bind to an inducer agent, such as a chemical agent. A non-limiting example of a pair of interaction domains (a first and second interaction domain) includes FRB protein and FKBP protein. The FK506 binding protein 1A (FKBP1A) (Gene ID: 2280) gene encodes the FKBP protein. The FKBP protein is a cis-trans prolyl isomerase enzyme that plays a role in immunoregulation and basic cellular processes involving protein folding and trafficking. FKBP also binds the immunosuppressants FK506 (tacrolimus) and rapamycin. The FKBP-rapamycin-binding (FRB) domain is the portion of the mTOR protein that interaction with rapamycin. Rapamycin binds the FRB domain of mTOR and inhibits its kinase activity.

Other non-limiting examples of interaction domains include GyrB, GAI, Calcineurin A, CyP-Fas, mTOR, Fab, BCL-xL, eDHFR, CRY2, LOV, PHYB, PIF, FKF1, GI, and Snap-Tag, and their corresponding binding partners, as well as those disclosed in Luker, K E et al. Proc Natl Acad Sci 2004 101(33): 12288-12293; Liang, F S, et al. Sci Signal 2011 4(164): rs2; Miyamoto, T, et al. Nat Chem Biol 2012 8: 465-470; Kennedy, M J, et al. Nat Methods 2012 7(12): 973-975; Yazawa, M, et al. Nat Biotechnol 2009 27(10): 941-945; Levskaya, A, et al., Nature 2009 461: 997-1001, the contents of which are incorporated herein in their entirety.

The iCASFx system enables greater control over splicing events by introducing an inducible component to the artificial RNA-guided splicing factors of the present disclosure. An inducer agent is an agent that promotes binding of two interaction domains to each other, or binding of two interaction domains to a third molecule, thereby bringing the two interaction domains into close proximity relative to each other. Non-limiting examples of agents which may be utilized in this system include chemicals (e.g., rapamycin, Coumermycin, or Gibberellin), light, and heat.

In some embodiments, an RNA splicing factor is fused to one interaction domain, and a catalytically inactive programmable nuclease is fused to another interaction domain. In some embodiments, an RNA splicing factor is fused to FRB, and a catalytically inactive programmable nuclease is fused to FKBP. In other embodiments, an RNA splicing factor is fused to FKBP, and a catalytically inactive programmable nuclease is fused to FRB.

The interaction domain may be used to the N-terminus or the C-terminus of the RNA splicing factor or the catalytically inactive programmable nuclease. In some embodiments, FRB is fused to the N-terminus of RBFOX1 or RBM38. In some embodiments, FRB is fused to the C-terminus of RBFOX1 or RBM38. In some embodiments, FRB is fused to the N-terminus of the catalytically inactive programmable nuclease. In some embodiments, FRB is fused to the C-terminus of the catalytically inactive programmable nuclease. In some embodiments, FKBP is fused to the N-terminus of RBFOX1 or RBM38. In some embodiments, FKBP is fused to the C-terminus of RBFOX1 or RBM38. In some embodiments, FKBP is fused to the N-terminus of the catalytically inactive programmable nuclease. In some embodiments, FKBP is fused to the C-terminus of the catalytically inactive programmable nuclease.

### Nucleic Acids and Vectors

Also provided are nucleic acids and vectors encoding any of the artificial RNA-guided splicing factors, complexes, or components thereof, as described herein. In some embodiments, the nucleic acid is DNA (e.g., in the form of a plasmid) or RNA (e.g., in the form of mRNA). As used herein, “vector” means a nucleic acid of any transmissible agent (e.g., plasmid or virus) into which nucleic acids encoding any of the artificial RNA-guided splicing factors, complexes, or components thereof can be spliced in order to introduce the nucleic acids(s) into host cells to promote its (their) replication and/or transcription.

In some embodiments, viral genomes comprising any of the foregoing nucleic acids (or sequences thereof) are provided. In some embodiments, the viral genome is in the form of an AAV genome (e.g., comprising inverted terminal repeats). In some embodiments, the viral genome (e.g., the AAV genome) is packaged in a viral particle (e.g., an AAV particle) capable of infecting/transducing a cell. Other forms of viral genomes and particles suitable for delivering the artificial RNA-guided splicing factors, complexes, or components thereof described herein are well known, and include, for example, adenovirus, AAV, HSV, Retroviruses (e.g., MMSV, MSCV), and Lentiviruses (e.g., HIV-1, HIV-2) (See e.g., Lundstrom, Diseases. 2018 June; 6(2): 42; the entire contents of which are hereby incorporated by reference).

SEQUENCES

>SEQ ID NO: 1, CUG (CONTROL GRNA)
GAACCCCUACCAACUGGUCGGGGUUUGAAACAGCAGCAGCAGCAGCAGCAGCAUUUUUUU
>SEQ ID NO: 2, SMN2-DN1 GRNA
GAACCCCUACCAACUGGUCGGGGUUUGAAACACAAAAGUAAGAUUCACUUUCAUUUUUUU
>SEQ ID NO: 3, SMN2-DN2 GRNA
GAACCCCUACCAACUGGUCGGGGUUUGAAACGAGAAUUCUAGUAGGGAUGUAGUUUUUUU
>SEQ ID NO: 4, SMN2-DN3 GRNA
GAACCCCUACCAACUGGUCGGGGUUUGAAACUUUCUUCCACACAACCAACCAGUUUUUUU
>SEQ ID NO: 5 SMN2-EX GRNA
GAACCCCUACCAACUGGUCGGGGUUUGAAACAAUGUGAGCACCUUCCUUCUUUUUUUUUU
>SEQ ID NO: 6 SMN2-UP1 GRNA
GAACCCCUACCAACUGGUCGGGGUUUGAAACGGCUGCAGUUAAGGUUUUCUUGUUUUUUU
>SEQ ID NO: 7 RG6-SA GRNA
GAACCCCUACCAACUGGUCGGGGUUUGAAACAUAUCGCCUGGAUCCUGAGCCAUUUUUUU
>SEQ ID NO: 8 DR-SMN2-2DR GRNA
GAACCCCUACCAACUGGUCGGGGUUUGAAACGAGAAUUCUAGUAGGGAUGUAGCAAGUAAACCCCUA
CCAACUGGUCGGGGUUUGAAACUUUUUUU
>SEQ ID NO: 9 DR-RG6-SA-DR
GAACCCCUACCAACUGGUCGGGGUUUGAAACAUAUCGCCUGGAUCCUGAGCCACAAGUAAACCCCUA
CCAACUGGUCGGGGUUUGAAACUUUUUUU
>SEQ ID NO: 10 SMN2-DN-RG6-SA
GAACCCCUACCAACUGGUCGGGGUUUGAAACACAAAAGUAAGAUUCACUUUCACAAGUAAACCCCUA
CCAACUGGUCGGGGUUUGAAACGAGAAUUCUAGUAGGGAUGUAGCAAGUAAACCCCUACCAACUGGU
CGGGGUUUGAAACUUUCUUCCACACAACCAACCAGCAAGUAAACCCCUACCAACUGGUCGGGGUUUG
AAACAUAUCGCCUGGAUCCUGAGCCAUUUUUUU
>SEQ ID NO: 11 SMN2-DN2-1XMS2
GAACCCCUACCAACUGGUCGGGGUUUGAAACGAGAAUUCUAGUAGGGAUGUAGCGAAUACGAGGGUC
UCCAGAUGGCCAACAUGAGGAUCACCCAUGUCUGCAGGGCCAGAUCUCGUAUUCGUUUUUUUU
>SEQ ID NO 12: SMN2-DN2-5XMS2B
GAACCCCUACCAACUGGUCGGGGUUUGAAACGAGAAUUCUAGUAGGGAUGUAGCGAAUACGAGGGUC
UCCAGAUGCGUACACCAUCAGGGUACGCAGAUGCGUACACCAUCAGGGUACGCAGAUGCGUACACCAU
CAGGGUACGCAGAUGCGUACACCAUCAGGGUACGCAGAUGCGUACACCAUCAGGGUACGCAGAUCUCG
UAUUCGUUUUUUUU
>SEQ ID NO: 13 DCASRX
MSPKKKRKVEASIEKKKSFAKGMGVKSTLVSGSKVYMTTFAEGSDARLEKIVEGDSIRSVNEGEAFSAEMAD
KNAGYKIGNAKFSHPKGYAVVANNPLYTGPVQQDMLGLKETLEKRYFGESADGNDNICIQVIHNILDIEKILAE
YITNAAYAVNNISGLDKDIIGFGKFSTVYTYDEFKDPEHHRAAFNNNDKLINAIKAQYDEFDNFLDNPRLGYFG
QAFFSKEGRNYIINYGNECYDILALLSGLAHWVVANNEEESRISRTWLYNLDKNLDNEYISTLNYLYDRITNEL
TNSFSKNSAANVNYIAETLGINPAEFAEQYFRFSIMKEQKNLGFNITKLREVMLDRKDMSEIRKNHKVFDSIRT
KVYTMMDFVIYRYYIEEDAKVAAANKSLPDNEKSLSEKDIFVINLRGSFNDDQKDALYYDEANRIWRKLENIM
HNIKEFRGNKTREYKKKDAPRLPRILPAGRDVSAFSKLMYALTMFLDGKEINDLLTTLINKFDNIQSFLKVMPLI
GVNAKFVEEYAFFKDSAKIADELRLIKSFARMGEPIADARRAMYIDAIRILGTNLSYDELKALADTFSLDENGN
KLKKGKHGMRNFIINNVISNKRFHYLIRYGDPAHLHEIAKNEAVVKFVLGRIADIQKKQGQNGKNQIDRYYET
CIGKDKGKSVSEKVDALTKIITGMNYDQFDKKRSVIEDTGRENAEREKFKKIISLYLTVIYHILKNIVNINARYVI
GFHCVERDAQLYKEKGYDINLKKLEEKGFSSVTKLCAGIDETAPDKRKDVEKEMAERAKESIDSLESANPKLY
ANYIKYSDEKKAEEFTRQINREKAKTALNAYLRNTKWNVIIREDLLRIDNKTCTLFANKAVALEVARYVHAYI
NDIAEVNSYFQLYHYIMQRIIMNERYEKSSGKVSEYFDAVNDEKKYNDRLLKLLCVPFGYCIPRFKNLSIEALF
DRNEAAKFDKEKKKVSGNSGSGPKKKRKVAAAYPYDVPDYA
>SEQ ID NO: 14 SV40NLS
PKKKRKV
>SEQ ID NO: 15 3XNLS
DPKKKRKVDPKKKRKVDPKKKRKV
>SEQ ID NO: 16 GGGGS LINKER
GGGGS
>SEQ ID NO: 17 GGGGS3XLINKER
GGGGSGGGGSGGGGS
>SEQ ID NO: 18 3XFLAG
MDYKDHDGDYKDHDIDYKDDDDK
>SEQ ID NO: 19 HA-TAG
YPYDVPDYA
>SEQ ID NO: 20 RBFOX1N-DCASRX-C [NP_061193.2(1-117) + DCASRX + NP_061193.2(190-397)]
MNCEREQLRGNQEAAAAPDTMAQPYASAQFAPPQNGIPAEYTAPHPHPAPEYTGQTTVPEHTLNLYPPAQTHS
EQSPADTSAQTVSGTATQTDDAAPTDGQPQTQPSENTENKSQPKGGGGSGRASPKKKRKVEASIEKKKSFAKG
MGVKSTLVSGSKVYMTTFAEGSDARLEKIVEGDSIRSVNEGEAFSAEMADKNAGYKIGNAKFSHPKGYAVVA
NNPLYTGPVQQDMLGLKETLEKRYFGESADGNDNICIQVIHNILDIEKILAEYITNAAYAVNNISGLDKDIIGFG
KFSTVYTYDEFKDPEHHRAAFNNNDKLINAIKAQYDEFDNFLDNPRLGYFGQAFFSKEGRNYIINYGNECYDIL
ALLSGLAHWVVANNEEESRISRTWLYNLDKNLDNEYISTLNYLYDRITNELTNSFSKNSAANVNYIAETLGINP
AEFAEQYFRFSIMKEQKNLGFNITKLREVMLDRKDMSEIRKNHKVFDSIRTKVYTMMDFVIYRYYIEEDAKVA
AANKSLPDNEKSLSEKDIFVINLRGSFNDDQKDALYYDEANRIWRKLENIMHNIKEFRGNKTREYKKKDAPRL
PRILPAGRDVSAFSKLMYALTMFLDGKEINDLLTTLINKFDNIQSFLKVMPLIGVNAKFVEEYAFFKDSAKIADE
LRLIKSFARMGEPIADARRAMYIDAIRILGTNLSYDELKALADTFSLDENGNKLKKGKHGMRNFIINNVISNKRF
HYLIRYGDPAHLHEIAKNEAVVKFVLGRIADIQKKQGQNGKNQIDRYYETCIGKDKGKSVSEKVDALTKIITG
MNYDQFDKKRSVIEDTGRENAEREKFKKIISLYLTVIYHILKNIVNINARYVIGFHCVERDAQLYKEKGYDINLK
KLEEKGFSSVTKLCAGIDETAPDKRKDVEKEMAERAKESIDSLESANPKLYANYIKYSDEKKAEEFTRQINREK
AKTALNAYLRNTKWNVIIREDLLRIDNKTCTLFANKAVALEVARYVHAYINDIAEVNSYFQLYHYIMQRIIMN
ERYEKSSGKVSEYFDAVNDEKKYNDRLLKLLCVPFGYCIPRFKNLSIEALFDRNEAAKFDKEKKKVSGNSGSG
PKKKRKVAAAYPYDVPDYAGGRGGGGSGGGGSGGGGSGPANATARVMTNKKTVNPYTNGWKLNPVVGAV
YSPEFYAGTVLLCQANQEGSSMYSAPSSLVYTSAMPGFPYPAATAAAAYRGAHLRGRGRTVYNTFRAAAPPP
PIPAYGGVVYQDGFYGADIYGGYAAYRYAQPTPATAAAYSDSYGRVYAADPYHHALAPAPTYGVGAMNAF
APLTDAKTRSHADDVGLVLSSLQASIYRGGYNRFAPY
>SEQ ID NO: 21 RBM38-DCASRX [NP_059965.2(1-239) + 3XNLS + GGGGS3XLINKER + DCASRX +
GGGGS3XLINKER + 3XFLAG]
MLLQPAPCAPSAGFPRPLAAPGAMHGSQKDTTFTKIFVGGLPYHTTDASLRKYFEGFGDIEEAVVITDRQTGKS
RGYGFVTMADRAAAERACKDPNPIIDGRKANVNLAYLGAKPRSLQTGFAIGVQQLHPTLIQRTYGLTPHYIYP
PAIVQPSVVIPAAPVPSLSSPYIEYTPASPAYAQYPPATYDQYPYAASPATAASFVGYSYPAAVPQALSAAAPAG
TTFVQYQAPQLQPDRMQNVIDGGGGSDPKKKRKVDPKKKRKVDPKKKRKVGSTGSRNDGGGGSGGGGSGG
GGSGRASPKKKRKVEASIEKKKSFAKGMGVKSTLVSGSKVYMTTFAEGSDARLEKIVEGDSIRSVNEGEAFSA
EMADKNAGYKIGNAKFSHPKGYAVVANNPLYTGPVQQDMLGLKETLEKRYFGESADGNDNICIQVIHNILDIE
KILAEYITNAAYAVNNISGLDKDIIGFGKFSTVYTYDEFKDPEHHRAAFNNNDKLINAIKAQYDEFDNFLDNPR
LGYFGQAFFSKEGRNYIINYGNECYDILALLSGLAHWVVANNEEESRISRTWLYNLDKNLDNEYISTLNYLYD
RITNELTNSFSKNSAANVNYIAETLGINPAEFAEQYFRFSIMKEQKNLGFNITKLREVMLDRKDMSEIRKNHKV
FDSIRTKVYTMMDFVIYRYYIEEDAKVAAANKSLPDNEKSLSEKDIFVINLRGSFNDDQKDALYYDEANRIWR
KLENIMHNIKEFRGNKTREYKKKDAPRLPRILPAGRDVSAFSKLMYALTMFLDGKEINDLLTTLINKFDNIQSFL
KVMPLIGVNAKFVEEYAFFKDSAKIADELRLIKSFARMGEPIADARRAMYIDAIRILGTNLSYDELKALADTFSL
DENGNKLKKGKHGMRNFIINNVISNKRFHYLIRYGDPAHLHEIAKNEAVVKFVLGRIADIQKKQGQNGKNQID
RYYETCIGKDKGKSVSEKVDALTKIITGMNYDQFDKKRSVIEDTGRENAEREKFKKIISLYLTVIYHILKNIVNIN
ARYVIGFHCVERDAQLYKEKGYDINLKKLEEKGFSSVTKLCAGIDETAPDKRKDVEKEMAERAKESIDSLESA
NPKLYANYIKYSDEKKAEEFTRQINREKAKTALNAYLRNTKWNVIIREDLLRIDNKTCTLFANKAVALEVARY
VHAYINDIAEVNSYFQLYHYIMQRIIMNERYEKSSGKVSEYFDAVNDEKKYNDRLLKLLCVPFGYCIPRFKNLS
IEALFDRNEAAKFDKEKKKVSGNSGSGPKKKRKVAAAYPYDVPDYAGGRGGGGSGGGGSGGGGSGPAMDY
KDHDGDYKDHDIDYKDDDDK
>SEQ ID NO: 22 DCASRX-RBM38 [3XFLAG + 3XNLS + GGGGS3XLINKER + DCASRX + GGGGS3XLINKER +
NP_059965.2(1-239)]
MDYKDHDGDYKDHDIDYKDDDDKIDGGGGSDPKKKRKVDPKKKRKVDPKKKRKVGSTGSRNDGGGGSGG
GGSGGGGSGRASPKKKRKVEASIEKKKSFAKGMGVKSTLVSGSKVYMTTFAEGSDARLEKIVEGDSIRSVNEG
EAFSAEMADKNAGYKIGNAKFSHPKGYAVVANNPLYTGPVQQDMLGLKETLEKRYFGESADGNDNICIQVIH
NILDIEKILAEYITNAAYAVNNISGLDKDIIGFGKFSTVYTYDEFKDPEHHRAAFNNNDKLINAIKAQYDEFDNF
LDNPRLGYFGQAFFSKEGRNYIINYGNECYDILALLSGLAHWVVANNEEESRISRTWLYNLDKNLDNEYISTLN
YLYDRITNELTNSFSKNSAANVNYIAETLGINPAEFAEQYFRFSIMKEQKNLGFNITKLREVMLDRKDMSEIRK
NHKVFDSIRTKVYTMMDFVIYRYYIEEDAKVAAANKSLPDNEKSLSEKDIFVINLRGSFNDDQKDALYYDEAN
RIWRKLENIMHNIKEFRGNKTREYKKKDAPRLPRILPAGRDVSAFSKLMYALTMFLDGKEINDLLTTLINKFDN
IQSFLKVMPLIGVNAKFVEEYAFFKDSAKIADELRLIKSFARMGEPIADARRAMYIDAIRILGTNLSYDELKALA
DTFSLDENGNKLKKGKHGMRNFIINNVISNKRFHYLIRYGDPAHLHEIAKNEAVVKFVLGRIADIQKKQGQNG
KNQIDRYYETCIGKDKGKSVSEKVDALTKIITGMNYDQFDKKRSVIEDTGRENAEREKFKKIISLYLTVIYHILK
NIVNINARYVIGFHCVERDAQLYKEKGYDINLKKLEEKGFSSVTKLCAGIDETAPDKRKDVEKEMAERAKESI
DSLESANPKLYANYIKYSDEKKAEEFTRQINREKAKTALNAYLRNTKWNVIIREDLLRIDNKTCTLFANKAVA
LEVARYVHAYINDIAEVNSYFQLYHYIMQRIIMNERYEKSSGKVSEYFDAVNDEKKYNDRLLKLLCVPFGYCI
PRFKNLSIEALFDRNEAAKFDKEKKKVSGNSGSGPKKKRKVAAAYPYDVPDYAGGRGGGGSGGGGSGGGGS
GPAMLLQPAPCAPSAGFPRPLAAPGAMHGSQKDTTFTKIFVGGLPYHTTDASLRKYFEGFGDIEEAVVITDRQT
GKSRGYGFVTMADRAAAERACKDPNPIIDGRKANVNLAYLGAKPRSLQTGFAIGVQQLHPTLIQRTYGLTPHY
IYPPAIVQPSVVIPAAPVPSLSSPYIEYTPASPAYAQYPPATYDQYPYAASPATAASFVGYSYPAAVPQALSAAA
PAGTTFVQYQAPQLQPDRMQ
>SEQ ID NO: 23 RBFOX1N-MCP-C [NP_061193.2(1-117) + MCP + NP_061193.2(190-397)]
MNCEREQLRGNQEAAAAPDTMAQPYASAQFAPPQNGIPAEYTAPHPHPAPEYTGQTTVPEHTLNLYPPAQTHS
EQSPADTSAQTVSGTATQTDDAAPTDGQPQTQPSENTENKSQPKGGGGSGRAMASNFTQFVLVDNGGTGDVT
VAPSNFANGVAEWISSNSRSQAYKVTCSVRQSSAQKRKYTIKVEVPKVATQTVGGVELPVAAWRSYLNMELT
IPIFATNSDCELIVKAMQGLLKDGNPIPSAIAANSGIYSAGGRGGGGSGGGGSGGGGSGPANATARVMTNKKT
VNPYTNGWKLNPVVGAVYSPEFYAGTVLLCQANQEGSSMYSAPSSLVYTSAMPGFPYPAATAAAAYRGAHL
RGRGRTVYNTFRAAAPPPPIPAYGGVVYQDGFYGADIYGGYAAYRYAQPTPATAAAYSDSYGRVYAADPYH
HALAPAPTYGVGAMNAFAPLTDAKTRSHADDVGLVLSSLQASIYRGGYNRFAPY
>SEQ ID NO: 24 DCASRX-DAZAP1(191-407)
[3XFLAG + 3XNLS + GGGGS3XLINKER + DCASRX + GGGGS3XLINKER + AAF78364.1(191-407)]
MDYKDHDGDYKDHDIDYKDDDDKIDGGGGSDPKKKRKVDPKKKRKVDPKKKRKVGSTGSRNDGGGGSGG
GGSGGGGSGRASPKKKRKVEASIEKKKSFAKGMGVKSTLVSGSKVYMTTFAEGSDARLEKIVEGDSIRSVNEG
EAFSAEMADKNAGYKIGNAKFSHPKGYAVVANNPLYTGPVQQDMLGLKETLEKRYFGESADGNDNICIQVIH
NILDIEKILAEYITNAAYAVNNISGLDKDIIGFGKFSTVYTYDEFKDPEHHRAAFNNNDKLINAIKAQYDEFDNF
LDNPRLGYFGQAFFSKEGRNYIINYGNECYDILALLSGLAHWVVANNEEESRISRTWLYNLDKNLDNEYISTLN
YLYDRITNELTNSFSKNSAANVNYIAETLGINPAEFAEQYFRFSIMKEQKNLGFNITKLREVMLDRKDMSEIRK
NHKVFDSIRTKVYTMMDFVIYRYYIEEDAKVAAANKSLPDNEKSLSEKDIFVINLRGSFNDDQKDALYYDEAN
RIWRKLENIMHNIKEFRGNKTREYKKKDAPRLPRILPAGRDVSAFSKLMYALTMFLDGKEINDLLTTLINKFDN
IQSFLKVMPLIGVNAKFVEEYAFFKDSAKIADELRLIKSFARMGEPIADARRAMYIDAIRILGTNLSYDELKALA
DTFSLDENGNKLKKGKHGMRNFIINNVISNKRFHYLIRYGDPAHLHEIAKNEAVVKFVLGRIADIQKKQGQNG
KNQIDRYYETCIGKDKGKSVSEKVDALTKIITGMNYDQFDKKRSVIEDTGRENAEREKFKKIISLYLTVIYHILK
NIVNINARYVIGFHCVERDAQLYKEKGYDINLKKLEEKGFSSVTKLCAGIDETAPDKRKDVEKEMAERAKESI
DSLESANPKLYANYIKYSDEKKAEEFTRQINREKAKTALNAYLRNTKWNVIIREDLLRIDNKTCTLFANKAVA
LEVARYVHAYINDIAEVNSYFQLYHYIMQRIIMNERYEKSSGKVSEYFDAVNDEKKYNDRLLKLLCVPFGYCI
PRFKNLSIEALFDRNEAAKFDKEKKKVSGNSGSGPKKKRKVAAAYPYDVPDYAGGRGGGGSGGGGSGGGGS
GPARDSKSQAPGQPGASQWGSRVVPNAANGWAGQPPPTWQQGYGPQGMWVPAGQAIGGYGPPPAGRGAPP
PPPPFTSYIVSTPPGGFPPPQGFPQGYGAPPQFSFGYGPPPPPPDQFAPPGVPPPPATPGAAPLAFPPPPSQAAPDM
SKPPTAQPDFPYGQYAGYGQDLSGFGQGFSDPSQQPPSYGGPSVPGSGGPPAGGSGFGRGQNHNVQGFHPYRR
>SEQ ID NO: 25 U2AF65-DCASRX [NP_001012496.1(1-471) + 3XNLS + GGGGS3XLINKER + DCASRX +
GGGGS3XLINKER + 3XFLAG]
MGMSDFDEFERQLNENKQERDKENRHRKRSHSRSRSRDRKRRSRSRDRRNRDQRSASRDRRRRSKPLTRGAK
EEHGGLIRSPRHEKKKKVRKYWDVPPPGFEHITPMQYKAMQAAGQIPATALLPTMTPDGLAVTPTPVPVVGSQ
MTRQARRLYVGNIPFGITEEAMMDFFNAQMRLGGLTQAPGNPVLAVQINQDKNFAFLEFRSVDETTQAMAFD
GIIFQGQSLKIRRPHDYQPLPGMSENPSVYVPGVVSTVVPDSAHKLFIGGLPNYLNDDQVKELLTSFGPLKAFNL
VKDSATGLSKGYAFCEYVDINVTDQAIAGLNGMQLGDKKLLVQRASVGAKNATLSTINQTPVTLQVPGLMSS
QVQMGGHPTEVLCLMNMVLPEELLDDEEYEEIVEDVRDECSKYGLVKSIEIPRPVDGVEVPGCGKIFVEFTSVF
DCQKAMQGLTGRKFANRVVVTKYCDPDSYHRRDFWNVIDGGGGSDPKKKRKVDPKKKRKVDPKKKRKVGS
TGSRNDGGGGSGGGGSGGGGSGRASPKKKRKVEASIEKKKSFAKGMGVKSTLVSGSKVYMTTFAEGSDARL
EKIVEGDSIRSVNEGEAFSAEMADKNAGYKIGNAKFSHPKGYAVVANNPLYTGPVQQDMLGLKETLEKRYFG
ESADGNDNICIQVIHNILDIEKILAEYITNAAYAVNNISGLDKDIIGFGKFSTVYTYDEFKDPEHHRAAFNNNDKL
INAIKAQYDEFDNFLDNPRLGYFGQAFFSKEGRNYIINYGNECYDILALLSGLAHWVVANNEEESRISRTWLYN
LDKNLDNEYISTLNYLYDRITNELTNSFSKNSAANVNYIAETLGINPAEFAEQYFRFSIMKEQKNLGFNITKLRE
VMLDRKDMSEIRKNHKVFDSIRTKVYTMMDFVIYRYYIEEDAKVAAANKSLPDNEKSLSEKDIFVINLRGSFN
DDQKDALYYDEANRIWRKLENIMHNIKEFRGNKTREYKKKDAPRLPRILPAGRDVSAFSKLMYALTMFLDGK
EINDLLTTLINKFDNIQSFLKVMPLIGVNAKFVEEYAFFKDSAKIADELRLIKSFARMGEPIADARRAMYIDAIRI
LGTNLSYDELKALADTFSLDENGNKLKKGKHGMRNFIINNVISNKRFHYLIRYGDPAHLHEIAKNEAVVKFVL
GRIADIQKKQGQNGKNQIDRYYETCIGKDKGKSVSEKVDALTKIITGMNYDQFDKKRSVIEDTGRENAEREKF
KKIISLYLTVIYHILKNIVNINARYVIGFHCVERDAQLYKEKGYDINLKKLEEKGFSSVTKLCAGIDETAPDKRK
DVEKEMAERAKESIDSLESANPKLYANYIKYSDEKKAEEFTRQINREKAKTALNAYLRNTKWNVIIREDLLRID
NKTCTLFANKAVALEVARYVHAYINDIAEVNSYFQLYHYIMQRIIMNERYEKSSGKVSEYFDAVNDEKKYND
RLLKLLCVPFGYCIPRFKNLSIEALFDRNEAAKFDKEKKKVSGNSGSGPKKKRKVAAAYPYDVPDYAGGRGG
GGSGGGGSGGGGSGPAMDYKDHDGDYKDHDIDYKDDDDK
>SEQ ID NO: 26 U2AF35A-DCASRX [NP_006749.1(1-240; L140I) + 3XNLS + GGGGS3XLINKER +
DCASRX + GGGGS3XLINKER + 3XFLAG]
MAEYLASIFGTEKDKVNCSFYFKIGACRHGDRCSRLHNKPTFSQTIALLNIYRNPQNSSQSADGLRCAVSDVEM
QEHYDEFFEEVFTEMEEKYGEVEEMNVCDNLGDHLVGNVYVKFRREEDAEKAVIDLNNRWFNGQPLHAELS
PVTDFREACCRQYEMGECTRGGFCNFMHLKPISRELRRELYGRRRKKHRSRSRSRERRSRSRDRGRGGGGGG
GGGGGGRERDRRRSRDRERSGRFNVIDGGGGSDPKKKRKVDPKKKRKVDPKKKRKVGSTGSRNDGGGGSGG
GGSGGGGSGRASPKKKRKVEASIEKKKSFAKGMGVKSTLVSGSKVYMTTFAEGSDARLEKIVEGDSIRSVNEG
EAFSAEMADKNAGYKIGNAKFSHPKGYAVVANNPLYTGPVQQDMLGLKETLEKRYFGESADGNDNICIQVIH
NILDIEKILAEYITNAAYAVNNISGLDKDIIGFGKFSTVYTYDEFKDPEHHRAAFNNNDKLINAIKAQYDEFDNF
LDNPRLGYFGQAFFSKEGRNYIINYGNECYDILALLSGLAHWVVANNEEESRISRTWLYNLDKNLDNEYISTLN
YLYDRITNELTNSFSKNSAANVNYIAETLGINPAEFAEQYFRFSIMKEQKNLGFNITKLREVMLDRKDMSEIRK
NHKVFDSIRTKVYTMMDFVIYRYYIEEDAKVAAANKSLPDNEKSLSEKDIFVINLRGSFNDDQKDALYYDEAN
RIWRKLENIMHNIKEFRGNKTREYKKKDAPRLPRILPAGRDVSAFSKLMYALTMFLDGKEINDLLTTLINKFDN
IQSFLKVMPLIGVNAKFVEEYAFFKDSAKIADELRLIKSFARMGEPIADARRAMYIDAIRILGTNLSYDELKALA
DTFSLDENGNKLKKGKHGMRNFIINNVISNKRFHYLIRYGDPAHLHEIAKNEAVVKFVLGRIADIQKKQGQNG
KNQIDRYYETCIGKDKGKSVSEKVDALTKIITGMNYDQFDKKRSVIEDTGRENAEREKFKKIISLYLTVIYHILK
NIVNINARYVIGFHCVERDAQLYKEKGYDINLKKLEEKGFSSVTKLCAGIDETAPDKRKDVEKEMAERAKESI
DSLESANPKLYANYIKYSDEKKAEEFTRQINREKAKTALNAYLRNTKWNVIIREDLLRIDNKTCTLFANKAVA
LEVARYVHAYINDIAEVNSYFQLYHYIMQRIIMNERYEKSSGKVSEYFDAVNDEKKYNDRLLKLLCVPFGYCI
PRFKNLSIEALFDRNEAAKFDKEKKKVSGNSGSGPKKKRKVAAAYPYDVPDYAGGRGGGGSGGGGSGGGGS
GPAMDYKDHDGDYKDHDIDYKDDDDK
>SEQ ID NO: 27 DCASRX-U2AF65
[3XFLAG + 3XNLS + GGGGS3XLINKER + DCASRX + GGGGS3XLINKER + NP_001012496.1(1-471; T350M)
MDYKDHDGDYKDHDIDYKDDDDKIDGGGGSDPKKKRKVDPKKKRKVDPKKKRKVGSTGSRNDGGGGSGG
GGSGGGGSGRASPKKKRKVEASIEKKKSFAKGMGVKSTLVSGSKVYMTTFAEGSDARLEKIVEGDSIRSVNEG
EAFSAEMADKNAGYKIGNAKFSHPKGYAVVANNPLYTGPVQQDMLGLKETLEKRYFGESADGNDNICIQVIH
NILDIEKILAEYITNAAYAVNNISGLDKDIIGFGKFSTVYTYDEFKDPEHHRAAFNNNDKLINAIKAQYDEFDNF
LDNPRLGYFGQAFFSKEGRNYIINYGNECYDILALLSGLAHWVVANNEEESRISRTWLYNLDKNLDNEYISTLN
YLYDRITNELTNSFSKNSAANVNYIAETLGINPAEFAEQYFRFSIMKEQKNLGFNITKLREVMLDRKDMSEIRK
NHKVFDSIRTKVYTMMDFVIYRYYIEEDAKVAAANKSLPDNEKSLSEKDIFVINLRGSFNDDQKDALYYDEAN
RIWRKLENIMHNIKEFRGNKTREYKKKDAPRLPRILPAGRDVSAFSKLMYALTMFLDGKEINDLLTTLINKFDN
IQSFLKVMPLIGVNAKFVEEYAFFKDSAKIADELRLIKSFARMGEPIADARRAMYIDAIRILGTNLSYDELKALA
DTFSLDENGNKLKKGKHGMRNFIINNVISNKRFHYLIRYGDPAHLHEIAKNEAVVKFVLGRIADIQKKQGQNG
KNQIDRYYETCIGKDKGKSVSEKVDALTKIITGMNYDQFDKKRSVIEDTGRENAEREKFKKIISLYLTVIYHILK
NIVNINARYVIGFHCVERDAQLYKEKGYDINLKKLEEKGFSSVTKLCAGIDETAPDKRKDVEKEMAERAKESI
DSLESANPKLYANYIKYSDEKKAEEFTRQINREKAKTALNAYLRNTKWNVIIREDLLRIDNKTCTLFANKAVA
LEVARYVHAYINDIAEVNSYFQLYHYIMQRIIMNERYEKSSGKVSEYFDAVNDEKKYNDRLLKLLCVPFGYCI
PRFKNLSIEALFDRNEAAKFDKEKKKVSGNSGSGPKKKRKVAAAYPYDVPDYAGGRGGGGSGGGGSGGGGS
GPAMSDFDEFERQLNENKQERDKENRHRKRSHSRSRSRDRKRRSRSRDRRNRDQRSASRDRRRRSKPLTRGA
KEEHGGLIRSPRHEKKKKVRKYWDVPPPGFEHITPMQYKAMQAAGQIPATALLPTMTPDGLAVTPTPVPVVGS
QMTRQARRLYVGNIPFGITEEAMMDFFNAQMRLGGLTQAPGNPVLAVQINQDKNFAFLEFRSVDETTQAMAF
DGIIFQGQSLKIRRPHDYQPLPGMSENPSVYVPGVVSTVVPDSAHKLFIGGLPNYLNDDQVKELLTSFGPLKAF
NLVKDSATGLSKGYAFCEYVDINVTDQAIAGLNGMQLGDKKLLVQRASVGAKNATLSTINQMPVTLQVPGL
MSSQVQMGGHPTEVLCLMNMVLPEELLDDEEYEEIVEDVRDECSKYGLVKSIEIPRPVDGVEVPGCGKIFVEFT
SVFDCQKAMQGLTGRKFANRVVVTKYCDPDSYHRRDFW
>SEQ ID NO: 28 DCASRX-U2AF35B [3XFLAG + 3XNLS + GGGGS3XLINKER + DCASRX +
GGGGS3XLINKER + NP_001020374.1(1-240)]
MDYKDHDGDYKDHDIDYKDDDDKIDGGGGSDPKKKRKVDPKKKRKVDPKKKRKVGSTGSRNDGGGGSGG
GGSGGGGSGRASPKKKRKVEASIEKKKSFAKGMGVKSTLVSGSKVYMTTFAEGSDARLEKIVEGDSIRSVNEG
EAFSAEMADKNAGYKIGNAKFSHPKGYAVVANNPLYTGPVQQDMLGLKETLEKRYFGESADGNDNICIQVIH
NILDIEKILAEYITNAAYAVNNISGLDKDIIGFGKFSTVYTYDEFKDPEHHRAAFNNNDKLINAIKAQYDEFDNF
LDNPRLGYFGQAFFSKEGRNYIINYGNECYDILALLSGLAHWVVANNEEESRISRTWLYNLDKNLDNEYISTLN
YLYDRITNELTNSFSKNSAANVNYIAETLGINPAEFAEQYFRFSIMKEQKNLGFNITKLREVMLDRKDMSEIRK
NHKVFDSIRTKVYTMMDFVIYRYYIEEDAKVAAANKSLPDNEKSLSEKDIFVINLRGSFNDDQKDALYYDEAN
RIWRKLENIMHNIKEFRGNKTREYKKKDAPRLPRILPAGRDVSAFSKLMYALTMFLDGKEINDLLTTLINKFDN
IQSFLKVMPLIGVNAKFVEEYAFFKDSAKIADELRLIKSFARMGEPIADARRAMYIDAIRILGTNLSYDELKALA
DTFSLDENGNKLKKGKHGMRNFIINNVISNKRFHYLIRYGDPAHLHEIAKNEAVVKFVLGRIADIQKKQGQNG
KNQIDRYYETCIGKDKGKSVSEKVDALTKIITGMNYDQFDKKRSVIEDTGRENAEREKFKKIISLYLTVIYHILK
NIVNINARYVIGFHCVERDAQLYKEKGYDINLKKLEEKGFSSVTKLCAGIDETAPDKRKDVEKEMAERAKESI
DSLESANPKLYANYIKYSDEKKAEEFTRQINREKAKTALNAYLRNTKWNVIIREDLLRIDNKTCTLFANKAVA
LEVARYVHAYINDIAEVNSYFQLYHYIMQRIIMNERYEKSSGKVSEYFDAVNDEKKYNDRLLKLLCVPFGYCI
PRFKNLSIEALFDRNEAAKFDKEKKKVSGNSGSGPKKKRKVAAAYPYDVPDYAGGRGGGGSGGGGSGGGGS
GPAMAEYLASIFGTEKDKVNCSFYFKIGACRHGDRCSRLHNKPTFSQTILIQNIYRNPQNSAQTADGSHCAVSD
VEMQEHYDEFFEEVFTEMEEKYGEVEEMNVCDNLGDHLVGNVYVKFRREEDAEKAVIDLNNRWFNGQPIHA
ELSPVTDFREACCRQYEMGECTRGGFCNFMHLKPISRELRRELYGRRRKKHRSRSRSRERRSRSRDRGRGGGG
GGGGGGGGRERDRRRSRDRERSGRF
>SEQ ID NO: 29 FKBP-DCASRX [FKBP + 3XNLS + GGGGS3XLINKER + DCASRX + GGGGS3XLINKER +
3XFLAG]
MGGGSSGGGQISYASRGGVQVETISPGDGRTFPKRGQTCVVHYTGMLEDGKKFDSSRDRNKPFKFMLGKQEV
IRGWEEGVAQMSVGQRAKLTISPDYAYGATGHPGIIPPHATLVFDVELLKLENVIDGGGGSDPKKKRKVDPKK
KRKVDPKKKRKVGSTGSRNDGGGGSGGGGSGGGGSGRASPKKKRKVEASIEKKKSFAKGMGVKSTLVSGSK
VYMTTFAEGSDARLEKIVEGDSIRSVNEGEAFSAEMADKNAGYKIGNAKFSHPKGYAVVANNPLYTGPVQQD
MLGLKETLEKRYFGESADGNDNICIQVIHNILDIEKILAEYITNAAYAVNNISGLDKDIIGFGKFSTVYTYDEFKD
PEHHRAAFNNNDKLINAIKAQYDEFDNFLDNPRLGYFGQAFFSKEGRNYIINYGNECYDILALLSGLAHWVVA
NNEEESRISRTWLYNLDKNLDNEYISTLNYLYDRITNELTNSFSKNSAANVNYIAETLGINPAEFAEQYFRFSIM
KEQKNLGFNITKLREVMLDRKDMSEIRKNHKVFDSIRTKVYTMMDFVIYRYYIEEDAKVAAANKSLPDNEKS
LSEKDIFVINLRGSFNDDQKDALYYDEANRIWRKLENIMHNIKEFRGNKTREYKKKDAPRLPRILPAGRDVSAF
SKLMYALTMFLDGKEINDLLTTLINKFDNIQSFLKVMPLIGVNAKFVEEYAFFKDSAKIADELRLIKSFARMGEP
IADARRAMYIDAIRILGTNLSYDELKALADTFSLDENGNKLKKGKHGMRNFIINNVISNKRFHYLIRYGDPAHL
HEIAKNEAVVKFVLGRIADIQKKQGQNGKNQIDRYYETCIGKDKGKSVSEKVDALTKIITGMNYDQFDKKRSV
IEDTGRENAEREKFKKIISLYLTVIYHILKNIVNINARYVIGFHCVERDAQLYKEKGYDINLKKLEEKGFSSVTKL
CAGIDETAPDKRKDVEKEMAERAKESIDSLESANPKLYANYIKYSDEKKAEEFTRQINREKAKTALNAYLRNT
KWNVIIREDLLRIDNKTCTLFANKAVALEVARYVHAYINDIAEVNSYFQLYHYIMQRIIMNERYEKSSGKVSEY
FDAVNDEKKYNDRLLKLLCVPFGYCIPRFKNLSIEALFDRNEAAKFDKEKKKVSGNSGSGPKKKRKVAAAYP
YDVPDYAGGRGGGGSGGGGSGGGGSGPAMDYKDHDGDYKDHDIDYKDDDDK
>SEQ ID NO: 30 DCASRX-FKBP [3XFLAG + 3XNLS + GGGGS3XLINKER + DCASRX + GGGGS3XLINKER +
FKBP]
MDYKDHDGDYKDHDIDYKDDDDKIDGGGGSDPKKKRKVDPKKKRKVDPKKKRKVGSTGSRNDGGGGSGG
GGSGGGGSGRASPKKKRKVEASIEKKKSFAKGMGVKSTLVSGSKVYMTTFAEGSDARLEKIVEGDSIRSVNEG
EAFSAEMADKNAGYKIGNAKFSHPKGYAVVANNPLYTGPVQQDMLGLKETLEKRYFGESADGNDNICIQVIH
NILDIEKILAEYITNAAYAVNNISGLDKDIIGFGKFSTVYTYDEFKDPEHHRAAFNNNDKLINAIKAQYDEFDNF
LDNPRLGYFGQAFFSKEGRNYIINYGNECYDILALLSGLAHWVVANNEEESRISRTWLYNLDKNLDNEYISTLN
YLYDRITNELTNSFSKNSAANVNYIAETLGINPAEFAEQYFRFSIMKEQKNLGFNITKLREVMLDRKDMSEIRK
NHKVFDSIRTKVYTMMDFVIYRYYIEEDAKVAAANKSLPDNEKSLSEKDIFVINLRGSFNDDQKDALYYDEAN
RIWRKLENIMHNIKEFRGNKTREYKKKDAPRLPRILPAGRDVSAFSKLMYALTMFLDGKEINDLLTTLINKFDN
IQSFLKVMPLIGVNAKFVEEYAFFKDSAKIADELRLIKSFARMGEPIADARRAMYIDAIRILGTNLSYDELKALA
DTFSLDENGNKLKKGKHGMRNFIINNVISNKRFHYLIRYGDPAHLHEIAKNEAVVKFVLGRIADIQKKQGQNG
KNQIDRYYETCIGKDKGKSVSEKVDALTKIITGMNYDQFDKKRSVIEDTGRENAEREKFKKIISLYLTVIYHILK
NIVNINARYVIGFHCVERDAQLYKEKGYDINLKKLEEKGFSSVTKLCAGIDETAPDKRKDVEKEMAERAKESI
DSLESANPKLYANYIKYSDEKKAEEFTRQINREKAKTALNAYLRNTKWNVIIREDLLRIDNKTCTLFANKAVA
LEVARYVHAYINDIAEVNSYFQLYHYIMQRIIMNERYEKSSGKVSEYFDAVNDEKKYNDRLLKLLCVPFGYCI
PRFKNLSIEALFDRNEAAKFDKEKKKVSGNSGSGPKKKRKVAAAYPYDVPDYAGGRGGGGSGGGGSGGGGS
GPAGGGSSGGGQISYASRGGVQVETISPGDGRTFPKRGQTCVVHYTGMLEDGKKFDSSRDRNKPFKFMLGKQ
EVIRGWEEGVAQMSVGQRAKLTISPDYAYGATGHPGIIPPHATLVFDVELLKLE
>SEQ ID NO: 31 RBFOX1N-FRB-C [NP_061193.2(1-117) + FRB + NP_061193.2(190-397)]
MNCEREQLRGNQEAAAAPDTMAQPYASAQFAPPQNGIPAEYTAPHPHPAPEYTGQTTVPEHTLNLYPPAQTHS
EQSPADTSAQTVSGTATQTDDAAPTDGQPQTQPSENTENKSQPKGGGGSGRAMEMWHEGLEEASRLYFGERN
VKGMFEVLEPLHAMMERGPQTLKETSFNQAYGRDLMEAQEWCRKYMKSGNVKDLTQAWDLYYHVFRRISK
QQISYASRGGGSSGGGGGGGSGGGGSGGGGSGPANATARVMTNKKTVNPYTNGWKLNPVVGAVYSPEFYA
GTVLLCQANQEGSSMYSAPSSLVYTSAMPGFPYPAATAAAAYRGAHLRGRGRTVYNTFRAAAPPPPIPAYGG
VVYQDGFYGADIYGGYAAYRYAQPTPATAAAYSDSYGRVYAADPYHHALAPAPTYGVGAMNAFAPLTDAK
TRSHADDVGLVLSSLQASIYRGGYNRFAPY
>SEQ ID NO: 32 RBM38-FRB [NP_059965.2(1-239) + 3XNLS + GGGGS3XLINKER + FRB +
GGGGS3XLINKER + 3XFLAG]
MLLQPAPCAPSAGFPRPLAAPGAMHGSQKDTTFTKIFVGGLPYHTTDASLRKYFEGFGDIEEAVVITDRQTGKS
RGYGFVTMADRAAAERACKDPNPIIDGRKANVNLAYLGAKPRSLQTGFAIGVQQLHPTLIQRTYGLTPHYIYP
PAIVQPSVVIPAAPVPSLSSPYIEYTPASPAYAQYPPATYDQYPYAASPATAASFVGYSYPAAVPQALSAAAPAG
TTFVQYQAPQLQPDRMQNVIDGGGGSDPKKKRKVDPKKKRKVDPKKKRKVGSTGSRNDGGGGSGGGGSGG
GGSGRAMEMWHEGLEEASRLYFGERNVKGMFEVLEPLHAMMERGPQTLKETSFNQAYGRDLMEAQEWCRK
YMKSGNVKDLTQAWDLYYHVFRRISKQQISYASRGGGSSGGGGGGGSGGGGSGGGGSGPAMDYKDHDGDY
KDHDIDYKDDDDK
>SEQ ID NO: 33 FRB-RBM38 [3XFLAG + 3XNLS + GGGGS3XLINKER + FRB + GGGGS3XLINKER +
NP_059965.2(1-239)]
MDYKDHDGDYKDHDIDYKDDDDKIDGGGGSDPKKKRKVDPKKKRKVDPKKKRKVGSTGSRNDGGGGSGG
GGSGGGGSGRAMEMWHEGLEEASRLYFGERNVKGMFEVLEPLHAMMERGPQTLKETSFNQAYGRDLMEAQ
EWCRKYMKSGNVKDLTQAWDLYYHVFRRISKQQISYASRGGGSSGGGGGGGSGGGGSGGGGSGPAMLLQP
APCAPSAGFPRPLAAPGAMHGSQKDTTFTKIFVGGLPYHTTDASLRKYFEGFGDIEEAVVITDRQTGKSRGYGF
VTMADRAAAERACKDPNPIIDGRKANVNLAYLGAKPRSLQTGFAIGVQQLHPTLIQRTYGLTPHYIYPPAIVQP
SVVIPAAPVPSLSSPYIEYTPASPAYAQYPPATYDQYPYAASPATAASFVGYSYPAAVPQALSAAAPAGTTFVQ
YQAPQLQPDRMQ
>SEQ ID NO: 34 PCR8-SGCASRX GRNA CLONING PLASMID
CTTTCCTGCGTTATCCCCTGATTCTGTGGATAACCGTATTACCGCCTTTGAGTGAGCTGATACCGCTCGCCG
CAGCCGAACGACCGAGCGCAGCGAGTCAGTGAGCGAGGAAGCGGAAGAGCGCCCAATACGCAAACCGC
CTCTCCCCGCGCGTTGGCCGATTCATTAATGCAGCTGGCACGACAGGTTTCCCGACTGGAAAGCGGGCAG
TGAGCGCAACGCAATTAATACGCGTACCGCTAGCCAGGAAGAGTTTGTAGAAACGCAAAAAGGCCATCC
GTCAGGATGGCCTTCTGCTTAGTTTGATGCCTGGCAGTTTATGGCGGGCGTCCTGCCCGCCACCCTCCGGG
CCGTTGCTTCACAACGATCAAATCCGCTCCCGGCGGATTTGTCCTACTCAGGAGAGCGTTCACCGACAAA
CAACAGATAAAACGAAAGGCCCAGTATTCCGACTGAGCCTTTCGTTTTATTTGATGCCTGGCAGTTCCCTA
CTCTCGCGTTAACGCTAGCATGGATGTTTTCCCAGTCACGACGTTGTAAAACGACGGCCAGTCTTAAGCTC
GGGCCCCAAATAATGATTTTATTTTGACTGATAGTGACCTGTTCGTTGCAACAAATTGATGAGCAATGCTT
TTTTATAATGCCAACTTTGTACAAAAAAGCAGGCTCCGAATTCACCGGTGAGGGCCTATTTCCCATGATTC
CTTCATATTTGCATATACGATACAAGGCTGTTAGAGAGATAATTGGAATTAATTTGACTGTAAACACAAA
GATATTAGTACAAAATACGTGACGTAGAAAGTAATAATTTCTTGGGTAGTTTGCAGTTTTAAAATTATGTT
TTAAAATGGACTATCATATGCTTACCGTAACTTGAAAGTATTTCGATTTCTTGGCTTTATATATCTTGTGGA
AAGGACGAAACACCGAACCCCTACCAACTGGTCGGGGTTTGAAACGGGTCTTCTCGACCTGCAGACTGGC
TGTGTATAAGGGAGCCTGACATTTATATTCCCCAGAACATCAGGTTAATGGCGTTTTTGATGTCATTTTCG
CGGTGGCTGAGATCAGCCACTTCTTCCCCGATAACGGACACCGGCACACTGGCCATATCGGTGGTCATCA
TGCGCCAGCTTTCATCCCCGATATGCACCACCGGGTAAAGTTCACGGGAGACTTTATCTGACAGCAGACG
TGCACTGGCCAGGGGGATCACCATCCGTCGCCCGGGCGTGTCAATAATATCACTCTGTACATCCACAAAC
AGACGATAACGGCTCTCTCTTTTATAGGTGTAAACCTTAAACTGCATTTCACCAGCCCCTGTTCTCGTCAG
CAAAAGAGCCGTTCATTTCAATAAACCGGGCGACCTCAGCCATCCCTTCCTGATTTTCCGCTTTCCAGCGT
TCGGCACGCAGACGACGGGCTTCATTCTGCATGGTTGTGCTTACCAGACCGGAGATATTGACATCATATAT
GCCTTGAGCAACTGATAGCTGTCGCTGTCAACTGTCACTGTAATACGCTGCTTCATAGCATACCTCTTTTT
GACATACTTCGGGTATACATATCAGTATATATTCTTATACCGCAAAAATCAGCGCGCAAATACGCATACT
GTTATCTGGCTTTTAGTAAGCCGGATCCAGATCTTTACGCCCCGCCCTGCCACTCATCGCAGTACTGTTGT
AATTCATTAAGCATTCTGCCGACATGGAAGCCATCACAAACGGCATGATGAACCTGAATCGCCAGCGGCA
TCAGCACCTTGTCGCCTTGCGTATAATATTTGCCCATGGTGAAAACGGGGGCGAAGAAGTTGTCCATATTG
GCCACGTTTAAATCAAAACTGGTGAAACTCACCCAGGGATTGGCTGACACGAAAAACATATTCTCAATAA
ACCCTTTAGGGAAATAGGCCAGGTTTTCACCGTAACACGCCACATCTTGCGAATATATGTGTAGAAACTG
CCGGAAATCGTCGTGGTATTCACTCCAGAGCGATGAAAAGGTTTCAGTTTGCTCATGGAAAACGGTGTAA
CAAGGGTGAACACTATCCCATATCACCAGCTCACCGTCTTTCATTGCCATACGGAATTCCGGATGAGCATT
CATCAGGCGGGCAAGAATGTGAATAAAGGCCGGATAAAACTTGTGCTTATTTTTCTTTACGGTCTTTAAAA
AGGCCGTAATATCCAGCTGAACGGTCTGGTTATAGGTACATTGAGCAACTGACTGAAATGCCTCAAAATG
TTCTTTACGATGCCATTGGGATATATCAACGGTGGTATATCCAGTGATTTTTTTCTCCATTTTAGCTTCCTT
AGCTCCTGAAAATCTCGACGGATCCTAACTCAAAATCCACACATTATACGAGCCGGAAGCATAAAGTGTA
AAGCCTGGGGTGCCTAATGCGGCCGCGAAGACCTTTTTTTTGGCGCGCCTTAATTAAGAATTCGACCCAGC
TTTCTTGTACAAAGTTGGCATTATAAAAAATAATTGCTCATCAATTTGTTGCAACGAACAGGTCACTATCA
GTCAAAATAAAATCATTATTTGCCATCCAGCTGATATCCCCTATAGTGAGTCGTATTACATGGTCATAGCT
GTTTCCTGGCAGCTCTGGCCCGTGTCTCAAAATCTCTGATGTTACATTGCACAAGATAAAAATATATCATC
ATGCCTCCTCTAGACCAGCCAGGACAGAAATGCCTCGACTTCGCTGCTGCCCAAGGTTGCCGGGTGACGC
ACACCGTGGAAACGGATGAAGGCACGAACCCAGTGGACATAAGCCTGTTCGGTTCGTAAGCTGTAATGCA
AGTAGCGTATGCGCTCACGCAACTGGTCCAGAACCTTGACCGAACGCAGCGGTGGTAACGGCGCAGTGGC
GGTTTTCATGGCTTGTTATGACTGTTTTTTTGGGGTACAGTCTATGCCTCGGGCATCCAAGCAGCAAGCGC
GTTACGCCGTGGGTCGATGTTTGATGTTATGGAGCAGCAACGATGTTACGCAGCAGGGCAGTCGCCCTAA
AACAAAGTTAAACATCATGAGGGAAGCGGTGATCGCCGAAGTATCGACTCAACTATCAGAGGTAGTTGG
CGTCATCGAGCGCCATCTCGAACCGACGTTGCTGGCCGTACATTTGTACGGCTCCGCAGTGGATGGCGGC
CTGAAGCCACACAGTGATATTGATTTGCTGGTTACGGTGACCGTAAGGCTTGATGAAACAACGCGGCGAG
CTTTGATCAACGACCTTTTGGAAACTTCGGCTTCCCCTGGAGAGAGCGAGATTCTCCGCGCTGTAGAAGTC
ACCATTGTTGTGCACGACGACATCATTCCGTGGCGTTATCCAGCTAAGCGCGAACTGCAATTTGGAGAAT
GGCAGCGCAATGACATTCTTGCAGGTATCTTCGAGCCAGCCACGATCGACATTGATCTGGCTATCTTGCTG
ACAAAAGCAAGAGAACATAGCGTTGCCTTGGTAGGTCCAGCGGCGGAGGAACTCTTTGATCCGGTTCCTG
AACAGGATCTATTTGAGGCGCTAAATGAAACCTTAACGCTATGGAACTCGCCGCCCGACTGGGCTGGCGA
TGAGCGAAATGTAGTGCTTACGTTGTCCCGCATTTGGTACAGCGCAGTAACCGGCAAAATCGCGCCGAAG
GATGTCGCTGCCGACTGGGCAATGGAGCGCCTGCCGGCCCAGTATCAGCCCGTCATACTTGAAGCTAGAC
AGGCTTATCTTGGACAAGAAGAAGATCGCTTGGCCTCGCGCGCAGATCAGTTGGAAGAATTTGTCCACTA
CGTGAAAGGCGAGATCACCAAGGTAGTCGGCAAATAACCCTCGAGCCACCCATGACCAAAATCCCTTAAC
GTGAGTTACGCGTCGTTCCACTGAGCGTCAGACCCCGTAGAAAAGATCAAAGGATCTTCTTGAGATCCTTT
TTTTCTGCGCGTAATCTGCTGCTTGCAAACAAAAAAACCACCGCTACCAGCGGTGGTTTGTTTGCCGGATC
AAGAGCTACCAACTCTTTTTCCGAAGGTAACTGGCTTCAGCAGAGCGCAGATACCAAATACTGTCCTTCTA
GTGTAGCCGTAGTTAGGCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAGACGATAGTTACCG
GATAAGGCGCAGCGGTCGGGCTGAACGGGGGGTTCGTGCACACAGCCCAGCTTGGAGCGAACGACCTAC
ACCGAACTGAGATACCTACAGCGTGAGCATTGAGAAAGCGCCACGCTTCCCGAAGGGAGAAAGGCGGAC
AGGTATCCGGTAAGCGGCAGGGTCGGAACAGGAGAGCGCACGAGGGAGCTTCCAGGGGGAAACGCCTGG
TATCTTTATAGTCCTGTCGGGTTTCGCCACCTCTGACTTGAGCGTCGATTTTTGTGATGCTCGTCAGGGGGG
CGGAGCCTATGGAAAAACGCCAGCAACGCGGCCTTTTTACGGTTCCTGGCCTTTTGCTGGCCTTTTGCTCA
CATGTT
>SEQ ID NO: 35 PUC19-SGCASRX-1XMS2 GRNA CLONING PLASMID
ATTGATTTAAAACTTCATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTTTGTTTG
CCGGATCAAGAGCTACCAACTCTTTTTCCGAAGGTAACTGGCTTCAGCAGAGCGCAGATACCAAATACTG
TTCTTCTAGTGTAGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGACTCAAGACGATA
GTTACCGGATAAGGCGCAGCGGTCGGGCTGAACGGGGGGTTCGTGCACACAGCCCAGCTTGGAGCGAAC
GACCTACACCGAACTGAGATACCTACAGCGTGAGCTATGAGAAAGCGCCACGCTTCCCGAAGGGAGAAA
GGCGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTGGCCTTTTGCTGGCC
TTTTGCTCAGCTAGCGAGGGCCTATTTCCCATGATTCCTTCATATTTGCATATACGATACAAGGCTGTTAG
AGAGATAATTGGAATTAATTTGACTGTAAACACAAAGATATTAGTACAAAATACGTGACGTAGAAAGTAA
TAATTTCTTGGGTAGTTTGCAGTTTTAAAATTATGTTTTAAAATGGACTATCATATGCTTACCGTAACTTGA
AAGTATTTCGATTTCTTGGCTTTATATATCTTGTGGAAAGGACGAAACACCGAACCCCTACCAACTGGTCG
GGGTTTGAAACGGGTCTTCTCGACCTGCAGACTGGCTGTGTATAAGGGAGCCTGACATTTATATTCCCCAG
AACATCAGGTTAATGGCGTTTTTGATGTCATTTTCGCGGTGGCTGAGATCAGCCACTTCTTCCCCGATAAC
GGACACCGGCACACTGGCCATATCGGTGGTCATCATGCGCCAGCTTTCATCCCCGATATGCACCACCGGG
TAAAGTTCACGGGAGACTTTATCTGACAGCAGACGTGCACTGGCCAGGGGGATCACCATCCGTCGCCCGG
GCGTGTCAATAATATCACTCTGTACATCCACAAACAGACGATAACGGCTCTCTCTTTTATAGGTGTAAACC
TTAAACTGCATTTCACCAGCCCCTGTTCTCGTCAGCAAAAGAGCCGTTCATTTCAATAAACCGGGCGACCT
CAGCCATCCCTTCCTGATTTTCCGCTTTCCAGCGTTCGGCACGCAGACGACGGGCTTCATTCTGCATGGTT
GTGCTTACCAGACCGGAGATATTGACATCATATATGCCTTGAGCAACTGATAGCTGTCGCTGTCAACTGTC
ACTGTAATACGCTGCTTCATAGCATACCTCTTTTTGACATACTTCGGGTATACATATCAGTATATATTCTTA
TACCGCAAAAATCAGCGCGCAAATACGCATACTGTTATCTGGCTTTTAGTAAGCCGGATCCAGATCTTTAC
GCCCCGCCCTGCCACTCATCGCAGTACTGTTGTAATTCATTAAGCATTCTGCCGACATGGAAGCCATCACA
AACGGCATGATGAACCTGAATCGCCAGCGGCATCAGCACCTTGTCGCCTTGCGTATAATATTTGCCCATG
GTGAAAACGGGGGCGAAGAAGTTGTCCATATTGGCCACGTTTAAATCAAAACTGGTGAAACTCACCCAGG
GATTGGCTGACACGAAAAACATATTCTCAATAAACCCTTTAGGGAAATAGGCCAGGTTTTCACCGTAACA
CGCCACATCTTGCGAATATATGTGTAGAAACTGCCGGAAATCGTCGTGGTATTCACTCCAGAGCGATGAA
AAGGTTTCAGTTTGCTCATGGAAAACGGTGTAACAAGGGTGAACACTATCCCATATCACCAGCTCACCGT
CTTTCATTGCCATACGGAATTCCGGATGAGCATTCATCAGGCGGGCAAGAATGTGAATAAAGGCCGGATA
AAACTTGTGCTTATTTTTCTTTACGGTCTTTAAAAAGGCCGTAATATCCAGCTGAACGGTCTGGTTATAGG
TACATTGAGCAACTGACTGAAATGCCTCAAAATGTTCTTTACGATGCCATTGGGATATATCAACGGTGGTA
TATCCAGTGATTTTTTTCTCCATTTTAGCTTCCTTAGCTCCTGAAAATCTCGACGGATCCTAACTCAAAATC
CACACATTATACGAGCCGGAAGCATAAAGTGTAAAGCCTGGGGTGCCTAATGCGGCCGCGAAGACAACG
AATACGAGGGTCTCCAGATGGCCAACATGAGGATCACCCATGTCTGCAGGGCCAGATCTCGTATTCGTTT
TTTTTGGCGCGCCGAATTCTGATGCGGTATTTTCTCCTTACGCATCTGTGCGGTATTTCACACCGCATACGT
CAAAGCAACCATAGTACGCGCCCTGTAGCGGCGCATTAAGCGCGGCGGGTGTGGTGGTTACGCGCAGCGT
GACCGCTACACTTGCCAGCGCCTTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCTTTACGGCACCTCG
ACCCCAAAAAACTTGATTTGGGTGATGGTTCACGTAGTGGGCCATCGCCCTGATAGACGGTTTTTCGCCCT
TTGACGTTGGAGTCCACGTTCTTTAATAGTGGACTCTTGTTCCAAACTGGAACAACACTCAACTCTATCTC
GGGCTATTCTTTTGATTTATAAGGGATTTTGCCGATTTCGGTCTATTGGTTAAAAAATGAGCTGATTTAAC
AAAAATTTAACGCGAATTTTAACAAAATATTAACGTTTACAATTTTATGGTGCACTCTCAGTACAATCTGC
TCTGATGCCGCATAGTTAAGCCAGCCCCGACACCCGCCAACACCCGCTGACGCGCCCTGACGGGCTTGTC
TGCTCCCGGCATCCGCTTACAGACAAGCTGTGACCGTCTCCGGGAGCTGCATGTGTCAGAGGTTTTCACCG
TCATCACCGAAACGCGCGAGACGAAAGGGCCTCGTGATACGCCTATTTTTATAGGTTAATGTCATGATAA
TAATGGTTTCTTAGACGTCAGGTGGCACTTTTCGGGGAAATGTGCGCGGAACCCCTATTTGTTTATTTTTCT
AAATACATTCAAATATGTATCCGCTCATGAGACAATAACCCTGATAAATGCTTCAATAATATTGAAAAAG
GAAGAGTATGAGTATTCAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCACGAGTGGGTTACATC
GAACTGGATCTCAACAGCGGTAAGATCCTTGAGAGTTTTCGCCCCGAAGAACGTTTTCCAATGATGAGCA
CTTTTAAAGTTCTGCTATGTGGCGCGGTATTATCCCGTATTGACGCCGGGCAAGAGCAACTCGGTCGCCGC
ATACACTATTCTCAGAATGACTTGGTTGAGTACTCACCAGTCACAGAAAAGCATCTTACGGATGGCATGA
CAGTAAGAGAATTATGCAGTGCTGCCATAACCATGAGTGATAACACTGCGGCCAACTTACTTCTGACAAC
GATCGGAGGACCGAAGGAGCTAACCGCTTTTTTGCACAACATGGGGGATCATGTAACTCGCCTTGATCGT
TGGGAACCGGAGCTGAATGAAGCCATACCAAACGACGAGCGTGACACCACGATGCCTGTAGCAATGGCA
ACAACGTTGCGCAAACTATTAACTGGCGAACTACTTACTCTAGCTTCCCGGCAACAATTAATAGACTGGA
TGGAGGCGGATAAAGTTGCAGGACCACTTCTGCGCTCGGCCCTTCCGGCTGGCTGGTTTATTGCTGATAAA
TCTGGAGCCGGTGAGCGTGGAAGCCGCGGTATCATTGCAGCACTGGGGCCAGATGGTAAGCCCTCCCGTA
TCGTAGTTATCTACACGACGGGGAGTCAGGCAACTATGGATGAACGAAATAGACAGATCGCTGAGATAG
GTGCCTCACTGATTAAGCATTGGTAACTGTCAGACCAAGTTTACTCATATATACTTTAG
>SEQ ID NO: 36 PUC19-SGCASRX-5XMS2 GRNA CLONING PLASMID
ATTGATTTAAAACTTCATTTTTAATTTAAAAGGATCTAGGTGAAGATCCTTTTTGATAATCTCATGACCAA
AATCCCTTAACGTGAGTTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTTTGTTTG
CCGGATCAAGAGCTACCAACTCTTTTTCCGAAGGTAACTGGCTTCAGCAGAGCGCAGATACCAAATACTG
TTCTTCTAGTGTAGCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGGACTCAAGACGATA
GTTACCGGATAAGGCGCAGCGGTCGGGCTGAACGGGGGGTTCGTGCACACAGCCCAGCTTGGAGCGAAC
GACCTACACCGAACTGAGATACCTACAGCGTGAGCTATGAGAAAGCGCCACGCTTCCCGAAGGGAGAAA
GGCGGACAGGTATCCGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTGGCCTTTTGCTGGCC
TTTTGCTCAGCTAGCGAGGGCCTATTTCCCATGATTCCTTCATATTTGCATATACGATACAAGGCTGTTAG
AGAGATAATTGGAATTAATTTGACTGTAAACACAAAGATATTAGTACAAAATACGTGACGTAGAAAGTAA
TAATTTCTTGGGTAGTTTGCAGTTTTAAAATTATGTTTTAAAATGGACTATCATATGCTTACCGTAACTTGA
AAGTATTTCGATTTCTTGGCTTTATATATCTTGTGGAAAGGACGAAACACCGAACCCCTACCAACTGGTCG
GGGTTTGAAACGGGTCTTCTCGACCTGCAGACTGGCTGTGTATAAGGGAGCCTGACATTTATATTCCCCAG
AACATCAGGTTAATGGCGTTTTTGATGTCATTTTCGCGGTGGCTGAGATCAGCCACTTCTTCCCCGATAAC
GGACACCGGCACACTGGCCATATCGGTGGTCATCATGCGCCAGCTTTCATCCCCGATATGCACCACCGGG
TAAAGTTCACGGGAGACTTTATCTGACAGCAGACGTGCACTGGCCAGGGGGATCACCATCCGTCGCCCGG
GCGTGTCAATAATATCACTCTGTACATCCACAAACAGACGATAACGGCTCTCTCTTTTATAGGTGTAAACC
TTAAACTGCATTTCACCAGCCCCTGTTCTCGTCAGCAAAAGAGCCGTTCATTTCAATAAACCGGGCGACCT
CAGCCATCCCTTCCTGATTTTCCGCTTTCCAGCGTTCGGCACGCAGACGACGGGCTTCATTCTGCATGGTT
GTGCTTACCAGACCGGAGATATTGACATCATATATGCCTTGAGCAACTGATAGCTGTCGCTGTCAACTGTC
ACTGTAATACGCTGCTTCATAGCATACCTCTTTTTGACATACTTCGGGTATACATATCAGTATATATTCTTA
TACCGCAAAAATCAGCGCGCAAATACGCATACTGTTATCTGGCTTTTAGTAAGCCGGATCCAGATCTTTAC
GCCCCGCCCTGCCACTCATCGCAGTACTGTTGTAATTCATTAAGCATTCTGCCGACATGGAAGCCATCACA
AACGGCATGATGAACCTGAATCGCCAGCGGCATCAGCACCTTGTCGCCTTGCGTATAATATTTGCCCATG
GTGAAAACGGGGGCGAAGAAGTTGTCCATATTGGCCACGTTTAAATCAAAACTGGTGAAACTCACCCAGG
GATTGGCTGACACGAAAAACATATTCTCAATAAACCCTTTAGGGAAATAGGCCAGGTTTTCACCGTAACA
CGCCACATCTTGCGAATATATGTGTAGAAACTGCCGGAAATCGTCGTGGTATTCACTCCAGAGCGATGAA
AAGGTTTCAGTTTGCTCATGGAAAACGGTGTAACAAGGGTGAACACTATCCCATATCACCAGCTCACCGT
CTTTCATTGCCATACGGAATTCCGGATGAGCATTCATCAGGCGGGCAAGAATGTGAATAAAGGCCGGATA
AAACTTGTGCTTATTTTTCTTTACGGTCTTTAAAAAGGCCGTAATATCCAGCTGAACGGTCTGGTTATAGG
TACATTGAGCAACTGACTGAAATGCCTCAAAATGTTCTTTACGATGCCATTGGGATATATCAACGGTGGTA
TATCCAGTGATTTTTTTCTCCATTTTAGCTTCCTTAGCTCCTGAAAATCTCGACGGATCCTAACTCAAAATC
CACACATTATACGAGCCGGAAGCATAAAGTGTAAAGCCTGGGGTGCCTAATGCGGCCGCGAAGACAACG
AATACGAGGGTCTCCAGATGCGTACACCATCAGGGTACGCAGATGCGTACACCATCAGGGTACGCAGATG
CGTACACCATCAGGGTACGCAGATGCGTACACCATCAGGGTACGCAGATGCGTACACCATCAGGGTACGC
AGATCTCGTATTCGTTTTTTTTGGCGCGCCGAATTCTGATGCGGTATTTTCTCCTTACGCATCTGTGCGGTA
TTTCACACCGCATACGTCAAAGCAACCATAGTACGCGCCCTGTAGCGGCGCATTAAGCGCGGCGGGTGTG
GTGGTTACGCGCAGCGTGACCGCTACACTTGCCAGCGCCTTAGCGCCCGCTCCTTTCGCTTTCTTCCCTTCC
TTTCTCGCCACGTTCGCCGGCTTTCCCCGTCAAGCTCTAAATCGGGGGCTCCCTTTAGGGTTCCGATTTAGT
GCTTTACGGCACCTCGACCCCAAAAAACTTGATTTGGGTGATGGTTCACGTAGTGGGCCATCGCCCTGATA
GACGGTTTTTCGCCCTTTGACGTTGGAGTCCACGTTCTTTAATAGTGGACTCTTGTTCCAAACTGGAACAA
CACTCAACTCTATCTCGGGCTATTCTTTTGATTTATAAGGGATTTTGCCGATTTCGGTCTATTGGTTAAAAA
ATGAGCTGATTTAACAAAAATTTAACGCGAATTTTAACAAAATATTAACGTTTACAATTTTATGGTGCACT
CTCAGTACAATCTGCTCTGATGCCGCATAGTTAAGCCAGCCCCGACACCCGCCAACACCCGCTGACGCGC
CCTGACGGGCTTGTCTGCTCCCGGCATCCGCTTACAGACAAGCTGTGACCGTCTCCGGGAGCTGCATGTGT
CAGAGGTTTTCACCGTCATCACCGAAACGCGCGAGACGAAAGGGCCTCGTGATACGCCTATTTTTATAGG
TTAATGTCATGATAATAATGGTTTCTTAGACGTCAGGTGGCACTTTTCGGGGAAATGTGCGCGGAACCCCT
ATTTGTTTATTTTTCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCCCTTTTTTGCGGCAT
TTTGCCTTCCTGTTTTTGCTCACCCAGAAACGCTGGTGAAAGTAAAAGATGCTGAAGATCAGTTGGGTGCA
CGAGTGGGTTACATCGAACTGGATCTCAACAGCGGTAAGATCCTTGAGAGTTTTCGCCCCGAAGAACGTT
TTCCAATGATGAGCACTTTTAAAGTTCTGCTATGTGGCGCGGTATTATCCCGTATTGACGCCGGGCAAGAG
CAACTCGGTCGCCGCATACACTATTCTCAGAATGACTTGGTTGAGTACTCACCAGTCACAGAAAAGCATC
TTACGGATGGCATGACAGTAAGAGAATTATGCAGTGCTGCCATAACCATGAGTGATAACACTGCGGCCAA
CTTACTTCTGACAACGATCGGAGGACCGAAGGAGCTAACCGCTTTTTTGCACAACATGGGGGATCATGTA
ACTCGCCTTGATCGTTGGGAACCGGAGCTGAATGAAGCCATACCAAACGACGAGCGTGACACCACGATGC
CTGTAGCAATGGCAACAACGTTGCGCAAACTATTAACTGGCGAACTACTTACTCTAGCTTCCCGGCAACA
ATTAATAGACTGGATGGAGGCGGATAAAGTTGCAGGACCACTTCTGCGCTCGGCCCTTCCGGCTGGCTGG
TTTATTGCTGATAAATCTGGAGCCGGTGAGCGTGGAAGCCGCGGTATCATTGCAGCACTGGGGCCAGATG
GTAAGCCCTCCCGTATCGTAGTTATCTACACGACGGGGAGTCAGGCAACTATGGATGAACGAAATAGACA
GATCGCTGAGATAGGTGCCTCACTGATTAAGCATTGGTAACTGTCAGACCAAGTTTACTCATATATACTTT
AG
>SEQ ID NO: 37 PCI-SMN2 PLASMID (HTTPS://WWW.ADDGENE.ORG/72287/)
TCAATATTGGCCATTAGCCATATTATTCATTGGTTATATAGCATAAATCAATATTGGCTATTGGCCATTGC
ATACGTTGTATCTATATCATAATATGTACATTTATATTGGCTCATGTCCAATATGACCGCCATGTTGGCATT
GATTATTGACTAGTTATTAATAGTAATCAATTACGGGGTCATTAGTTCATAGCCCATATATGGAGTTCCGC
GTTACATAACTTACGGTAAATGGCCCGCCTGGCTGACCGCCCAACGACCCCCGCCCATTGACGTCAATAA
TGACGTATGTTCCCATAGTAACGCCAATAGGGACTTTCCATTGACGTCAATGGGTGGAGTATTTACGGTAA
ACTGCCCACTTGGCAGTACATCAAGTGTATCATATGCCAAGTCCGCCCCCTATTGACGTCAATGACGGTAA
ATGGCCCGCCTGGCATTATGCCCAGTACATGACCTTACGGGACTTTCCTACTTGGCAGTACATCTACGTAT
TAGTCATCGCTATTACCATGGTGATGCGGTTTTGGCAGTACACCAATGGGCGTGGATAGCGGTTTGACTCA
CGGGGATTTCCAAGTCTCCACCCCATTGACGTCAATGGGAGTTTGTTTTGGCACCAAAATCAACGGGACTT
TCCAAAATGTCGTAATAACCCCGCCCCGTTGACGCAAATGGGCGGTAGGCGTGTACGGTGGGAGGTCTAT
ATAAGCAGAGCTCGTTTAGTGAACCGTCAGATCACTAGAAGCTTTATTGCGGTAGTTTATCACAGTTAAAT
TGCTAACGCAGTCAGTGCTTCTGACACAACAGTCTCGAACTTAAGCTGCAGAAGTTGGTCGTGAGGCACT
GGGCAGGTAAGTATCAAGGTTACAAGACAGGTTTAAGGAGACCAATAGAAACTGGGCTTGTCGAGACAG
AGAAGACTCTTGCGTTTCTGATAGGCACCTATTGGTCTTACTGACATCCACTTTGCCTTTCTCTCCACAGGT
GTCCACTCCCAGTTCAATTACAGCTCTTAAGGCTAGAGTACTTAATACGACTCACTATAGGCTAGCCTCGA
GATAATTCCCCCACCACCTCCCATATGTCCAGATTCTCTTGATGATGCTGATGCTTTGGGAAGTATGTTAA
TTTCATGGTACATGAGTGGCTATCATACTGGCTATTATATGGTAAGTAATCACTCAGCATCTTTTCCTGAC
AATTTTTTTGTAGTTATGTGACTTTGTTTTGTAAATTTATAAAATACTACTTGCTTCTCTCTTTATATTACTA
AAAAATAAAAATAAAAAAATACAACTGTCTGAGGCTTAAATTACTCTTGCATTGTCCCTAAGTATAATTTT
AGTTAATTTTAAAAAGCTTTCATGCTATTGTTAGATTATTTTGATTATACACTTTTGAATTGAAATTATACT
TTTTCTAAATAATGTTTTAATCTCTGATTTGAAATTGATTGTAGGGAATGGAAAAGATGGGATAATTTTTC
ATAAATGAAAAATGAAATTCTTTTTTTTTTTTTTTTTTTTTTGAGACGGAGTCTTGCTCTGTTGCCCAGGCT
GGAGTGCAATGGCGTGATCTTGGCTCACAGCAAGCTCTGCCTCCTGGATTCACGCCATTCTCCTGCCTCAG
CCTCAGAGGTAGCTGGGACTACAGGTGCCTGCCACCACGCCTGTCTAATTTTTTGTATTTTTTTGTAAAGA
CAGGGTTTCACTGTGTTAGCCAGGATGGTCTCAATCTCCTGACCCCGTGATCCACCCGCCTCGGCCTTCCA
AGAGAAATGAAATTTTTTTAATGCACAAAGATCTGGGGTAATGTGTACCACATTGAACCTTGGGGAGTAT
GGCTTCAAACTTGTCACTTTATACGTTAGTCTCCTACGGACATGTTCTATTGTATTTTAGTCAGAACATTTA
AAATTATTTTATTTTATTTTATTTTTTTTTTTTTTTTGAGACGGAGTCTCGCTCTGTCACCCAGGCTGGAGTA
CAGTGGCGCAGTCTCGGCTCACTGCAAGCTCCGCCTCCCGGGTTCACGCCATTCTCCTGCCTCAGCCTCTC
CGAGTAGCTGGGACTACAGGCGCCCGCCACCACGCCCGGCTAATTTTTTTTTATTTTTAGTAGAGACGGGG
TTTCACCGTGGTCTCGATCTCCTGACCTCGTGATCCACCCGCCTCGGCCTCCCAAAGTGCTGGGATTACAA
GCGTGAGCCACCGCGCCCGGCCTAAAATTATTTTTAAAAGTAAGCTCTTGTGCCCTGCTAAAATTATGATG
TGATATTGTAGGCACTTGTATTTTTAGTAAATTAATATAGAAGAAACAACTGACTTAAAGGTGTATGTTTT
TAAATGTATCATCTGTGTGTGCCCCCATTAATATTCTTATTTAAAAGTTAAGGCCAGACATGGTGGCTTAC
AACTGTAATCCCAACAGTTTGTGAGGCCGAGGCAGGCAGATCACTTGAGGTCAGGAGTTTGAGACCAGCC
TGGCCAACATGATGAAACCTTGTCTCTACTAAAAATACCAAAAAAAATTTAGCCAGGCATGGTGGCACAT
GCCTGTAATCCGAGCTACTTGGGAGGCTGTGGCAGGAAAATTGCTTTAATCTGGGAGGCAGAGGTTGCAG
TGAGTTGAGATTGTGCCACTGCACTCCACCCTTGGTGACAGAGTGAGATTCCATCTCAAAAAAAGAAAAA
GGCCTGGCACGGTGGCTCACACCTATAATCCCAGTACTTTGGGAGGTAGAGGCAGGTGGATCACTTGAGG
TTAGGAGTTCAGGACCAGCCTGGCCAACATGGTGACTACTCCATTTCTACTAAATACACAAAACTTAGCC
CAGTGGCGGGCAGTTGTAATCCCAGCTACTTGAGAGGTTGAGGCAGGAGAATCACTTGAACCTGGGAGGC
AGAGGTTGCAGTGAGCCGAGATCACACCGCTGCACTCTAGCCTGGCCAACAGAGTGAGAATTTGCGGAG
GGAAAAAAAAGTCACGCTTCAGTTGTTGTAGTATAACCTTGGTATATTGTATGTATCATGAATTCCTCATT
TTAATGACCAAAAAGTAATAAATCAACAGCTTGTAATTTGTTTTGAGATCAGTTATCTGACTGTAACACTG
TAGGCTTTTGTGTTTTTTAAATTATGAAATATTTGAAAAAAATACATAATGTATATATAAAGTATTGGTAT
AATTTATGTTCTAAATAACTTTCTTGAGAAATAATTCACATGGTGTGCAGTTTACCTTTGAAAGTATACAA
GTTGGCTGGGCACAATGGCTCACGCCTGTAATCCCAGCACTTTGGGAGGCCAGGGCAGGTGGATCACGAG
GTCAGGAGATCGAGACCATCCTGGCTAACATGGTGAAACCCCGTCTCTACTAAAAGTACAAAAACAAATT
AGCCGGGCATGTTGGCGGGCACCTTTTGTCCCAGCTGCTCGGGAGGCTGAGGCAGGAGAGTGGCGTGAAC
CCAGGAGGTGGAGCTTGCAGTGAGCCGAGATTGTGCCAGTGCACTCCAGCCTGGGCGACAGAGCGAGAC
TCTGTCTCAAAAAATAAAATAAAAAAGAAAGTATACAAGTCAGTGGTTTTGGTTTTCAGTTATGCAACCA
TCACTACAATTTAAGAACATTTTCATCACCCCAAAAAGAAACCCTGTTACCTTCATTTTCCCCAGCCCTAG
GCAGTCAGTACACTTTCTGTCTCTATGAATTTGTCTATTTTAGATATTATATATAAACGGAATTATACGATA
TGTGGTCTTTTGTGTCTGGCTTCTTTCACTTAGCATGCTATTTTCAAGATTCATCCATGCTGTAGAATGCAC
CAGTACTGCATTCCTTCTTATTGCTGAATATTCTGTTGTTTGGTTATATCACATTTTATCCATTCATCAGTTC
ATGGACATTTAGGTTGTTTTTATTTTTGGGCTATAATGAATAATGTTGCTATGAACATTCGTTTGTGTTCTT
TTTGTTTTTTTGGTTTTTTGGGTTTTTTTTGTTTTGTTTTTGTTTTTGAGACAGTCTTGCTCTGTCTCCTAAGC
TGGAGTGCAGTGGCATGATCTTGGCTTACTGCAAGCTCTGCCTCCCGGGTTCACACCATTCTCCTGCCTCA
GCCCGACAAGTAGCTGGGACTACAGGCGTGTGCCACCATGCACGGCTAATTTTTTGTATTTTTAGTAGAGA
TGGGGTTTCACCGTGTTAGCCAGGATGGTCTCGATCTCCTGACCTCGTGATCTGCCTGCCTAGGCCTCCCA
AAGTGCTGGGATTACAGGCGTGAGCCACTGCACCTGGCCTTAAGTGTTTTTAATACGTCATTGCCTTAAGC
TAACAATTCTTAACCTTTGTTCTACTGAAGCCACGTGGTTGAGATAGGCTCTGAGTCTAGCTTTTAACCTCT
ATCTTTTTGTCTTAGAAATCTAAGCAGAATGCAAATGACTAAGAATAATGTTGTTGAAATAACATAAAAT
AGGTTATAACTTTGATACTCATTAGTAACAAATCTTTCAATACATCTTACGGTCTGTTAGGTGTAGATTAG
TAATGAAGTGGGAAGCCACTGCAAGCTAGTATACATGTAGGGAAAGATAGAAAGCATTGAAGCCAGAAG
AGAGACAGAGGACATTTGGGCTAGATCTGACAAGAAAAACAAATGTTTTAGTATTAATTTTTGACTTTAA
ATTTTTTTTTTATTTAGTGAATACTGGTGTTTAATGGTCTCATTTTAATAAGTATGACACAGGTAGTTTAAG
GTCATATATTTTATTTGATGAAAATAAGGTATAGGCCGGGCACGGTGGCTCACACCTGTAATCCCAGCACT
TTGGGAGGCCGAGGCAGGCGGATCACCTGAGGTCGGGAGTTAGAGACTAGCCTCAACATGGAGAAACCC
CGTCTCTACTAAAAAAAATACAAAATTAGGCGGGCGTGGTGGTGCATGCCTGTAATCCCAGCTACTCAGG
AGGCTGAGGCAGGAGAATTGCTTGAACCTGGGAGGTGGAGGTTGCGGTGAGCCGAGATCACCTCATTGC
ACTCCAGCCTGGGCAACAAGAGCAAAACTCCATCTCAAAAAAAAAAAAATAAGGTATAAGCGGGCTCAG
GAACATCATTGGACATACTGAAAGAAGAAAAATCAGCTGGGCGCAGTGGCTCACGCCGGTAATCCCAAC
ACTTTGGGAGGCCAAGGCAGGCGAATCACCTGAAGTCGGGAGTTCCAGATCAGCCTGACCAACATGGAG
AAACCCTGTCTCTACTAAAAATACAAAACTAGCCGGGCATGGTGGCGCATGCCTGTAATCCCAGCTACTT
GGGAGGCTGAGGCAGGAGAATTGCTTGAACCGAGAAGGCGGAGGTTGCGGTGAGCCAAGATTGCACCAT
TGCACTCCAGCCTGGGCAACAAGAGCGAAACTCCGTCTCAAAAAAAAAAGGAAGAAAAATATTTTTTTAA
ATTAATTAGTTTATTTATTTTTTAAGATGGAGTTTTGCCCTGTCACCCAGGCTGGGGTGCAATGGTGCAAT
CTCGGCTCACTGCAACCTCCGCCTCCTGGGTTCAAGTGATTCTCCTGCCTCAGCTTCCCGAGTAGCTGTGA
TTACAGCCATATGCCACCACGCCCAGCCAGTTTTGTGTTTTGTTTTGTTTTTTGTTTTTTTTTTTTGAGAGGG
TGTCTTGCTCTGTCCCCCAAGCTGGAGTGCAGCGGCGCGATCTTGGCTCACTGCAAGCTCTGCCTCCCAGG
TTCACACCATTCTCTTGCCTCAGCCTCCCGAGTAGCTGGGACTACAGGTGCCCGCCACCACACCCGGCTAA
TTTTTTTGTGTTTTTAGTAGAGATGGGGTTTCACTGTGTTAGCCAGGATGGTCTCGATCTCCTGACCTTTTG
ATCCACCCGCCTCAGCCTCCCCAAGTGCTGGGATTATAGGCGTGAGCCACTGTGCCCGGCCTAGTCTTGTA
TTTTTAGTAGAGTCGGGATTTCTCCATGTTGGTCAGGCTGTTCTCCAAATCCGACCTCAGGTGATCCGCCC
GCCTTGGCCTCCAAAAGTGCAAGGCAAGGCATTACAGGCATGAGCCACTGTGACCGGCAATGTTTTTAAA
TTTTTTACATTTAAATTTTATTTTTTAGAGACCAGGTCTCACTCTATTGCTCAGGCTGGAGTGCAAGGGCAC
ATTCACAGCTCACTGCAGCCTTGACCTCCAGGGCTCAAGCAGTCCTCTCACCTCAGTTTCCCGAGTAGCTG
GGACTACAGTGATAATGCCACTGCACCTGGCTAATTTTTATTTTTATTTATTTATTTTTTTTTGAGACAGAG
TCTTGCTCTGTCACCCAGGCTGGAGTGCAGTGGTGTAAATCTCAGCTCACTGCAGCCTCCGCCTCCTGGGT
TCAAGTGATTCTCCTGCCTCAACCTCCCAAGTAGCTGGGATTAGAGGTCCCCACCACCATGCCTGGCTAAT
TTTTTGTACTTTCAGTAGAAACGGGGTTTTGCCATGTTGGCCAGGCTGTTCTCGAACTCCTGAGCTCAGGT
GATCCAACTGTCTCGGCCTCCCAAAGTGCTGGGATTACAGGCGTGAGCCACTGTGCCTAGCCTGAGCCAC
CACGCCGGCCTAATTTTTAAATTTTTTGTAGAGACAGGGTCTCATTATGTTGCCCAGGGTGGTGTCAAGCT
CCAGGTCTCAAGTGATCCCCCTACCTCCGCCTCCCAAAGTTGTGGGATTGTAGGCATGAGCCACTGCAAG
AAAACCTTAACTGCAGCCTAATAATTGTTTTCTTTGGGATAACTTTTAAAGTACATTAAAAGACTATCAAC
TTAATTTCTGATCATATTTTGTTGAATAAAATAAGTAAAATGTCTTGTGAAACAAAATGCTTTTTAACATC
CATATAAAGCTATCTATATATAGCTATCTATATCTATATAGCTATTTTTTTTAACTTCCTTTATTTTCCTTAC
AGGGTTTTAGACAAAATCAAAAAGAAGGAAGGTGCTCACATTCCTTAAATTAAGGAGTAAGTCTGCCAGC
ATTATGAAAGTGAATCTTACTTTTGTAAAACTTTATGGTTTGTGGAAAACAAATGTTTTTGAACATTTAAA
AAGTTCAGATGTTAGAAAGTTGAAAGGTTAATGTAAAACAATCAATATTAAAGAATTTTGATGCCAAAAC
TATTAGATAAAAGGTTAATCTACATCCCTACTAGAATTCTCATACTTAACTGGTTGGTTGTGTGGAAGAAA
CATACTTTCACAATAAAGAGCTTTAGGATATGATGCCATTTTATATCACTAGTAGGCAGACCAGCAGACTT
TTTTTTATTGTGATATGGGATAACCTAGGCATACTGCACTGTACACTCTGACATATGAAGTGCTCTAGTCA
AGTTTAACTGGTGTCCACAGAGGACATGGTTTAACTGGAATTCGTCAAGCCTCTGGTTCTAATTTCTCATT
TGCAGGAAATGCTGGCATAGAGCAGCACTAAATGACACCACTAAAGAAACGATCAGACAGATCTGGAAT
GTGAAGCGTTATAGAAGATAACTGGCCTCATTTCTTCAAAATATCAAGTGTTGGGAAAGAAAAAAGGAAG
TGGAATGGGTAACTCTTCTTGATTAAAAGTTATGTAATAACCAAATGCAATGTGAAATATTTTACTGGACT
CTATTTTGAAAAACCATCTGTAAAAGACTGAGGTGGGGGTGGGAGGCCAGCACGGTGGTGAGGCAGTTG
AGAAAATTTGAATGTGGATTAGATTTTGAATGATATTGGATAATTATTGGTAATTTTATGAGCTGTGAGAA
GGGTGTTGTAGTTTATAAAAGACTGTCTTAATTTGCATACTTAAGCATTTAGGAATGAAGTGTTAGAGTGT
CTTAAAATGTTTCAAATGGTTTAACAAAATGTATGTGAGGCGTATGTGCCCGGGCGGCCGCTTCGAGCAG
ACATGATAAGATACATTGATGAGTTTGGACAAACCACAACTAGAATGCAGTGAAAAAAATGCTTTATTTG
TGAAATTTGTGATGCTATTGCTTTATTTGTAACCATTATAAGCTGCAATAAACAAGTTAACAACAACAATT
GCATTCATTTTATGTTTCAGGTTCAGGGGGAGATGTGGGAGGTTTTTTAAAGCAAGTAAAACCTCTACAAA
TGTGGTAAAATCGATAAGGATCCGGGCTGGCGTAATAGCGAAGAGGCCCGCACCGATCGCCCTTCCCAAC
AGTTGCGCAGCCTGAATGGCGAATGGACGCGCCCTGTAGCGGCGCATTAAGCGCGGCGGGTGTGGTGGTT
ACGCGCAGCGTGACCGCTACACTTGCCAGCGCCCTAGCGCCCGCTCCTTTCGCTTTCTTCCCTTCCTTTCTC
GCCACGTTCGCCGGCTTTCCCCGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTGGGCCATCGCCCTGATAGACG
GTTTTTCGCCCTTTGACGTTGGAGTCCACGTTCTTTAATAGTGGACTCTTGTTCCAAACTGGAACAACACTC
AACCCTATCTCGGTCTATTCTTTTGATTTATAAGGGATTTTGCCGATTTCGGCCTATTGGTTAAAAAATGAG
CTGATTTAACAAAAATTTAACGCGAATTTTAACAAAATATTAACGCTTACAATTTCCTGATGCGGTATTTT
CTCCTTACGCATCTGTGCGGTATTTCACACCGCATATGGTGCACTCTCAGTACAATCTGCTCTGATGCCGC
ATAGTTAAGCCAGCCCCGACACCCGCCAACACCCGCTGACGCGCCCTGACGGGCTTGTCTGCTCCCGGCA
TCCGCTTACAGACAAGCTGTGACCGTCTCCGGGAGCTGCATGTGTCAGAGGTTTTCACCGTCATCACCGAA
ACGCGCGAGACGAAAGGGCCTCGTGATACGCCTATTTTTATAGGTTAATGTCATGATAATAATGGTTTCTT
AGACGTCAGGTGGCACTTTTCGGGGAAATGTGCGCGGAACCCCTATTTGTTTATTTTTCTAAATACATTCA
AATATGTATCCGCTCATGAGACAATAACCCTGATAAATGCTTCAATAATATTGAAAAAGGAAGAGTATGA
GTATTCAACATTTCCGTGTCGCCCTTATTCCCTTTTTTGCGGCATTTTGCCTTCCTGTTTTTGCTCACCCAGA
AACGCTGGTGAAAGTAAAAGATGCTGAAGATCAGTTGGGTGCACGAGTGGGTTACATCGAACTGGATCTC
AACAGCGGTAAGATCCTTGAGAGTTTTCGCCCCGAAGAACGTTTTCCAATGATGAGCACTTTTAAAGTTCT
GCTATGTGGCGCGGTATTATCCCGTATTGACGCCGGGCAAGAGCAACTCGGTCGCCGCATACACTATTCTC
AGAATGACTTGGTTGAGTACTCACCAGTCACAGAAAAGCATCTTACGGATGGCATGACAGTAAGAGAATT
ATGCAGTGCTGCCATAACCATGAGTGATAACACTGCGGCCAACTTACTTCTGACAACGATCGGAGGACCG
AAGGAGCTAACCGCTTTTTTGCACAACATGGGGGATCATGTAACTCGCCTTGATCGTTGGGAACCGGAGC
TGAATGAAGCCATACCAAACGACGAGCGTGACACCACGATGCCTGTAGCAATGGCAACAACGTTGCGCA
AACTATTAACTGGCGAACTACTTACTCTAGCTTCCCGGCAACAATTAATAGACTGGATGGAGGCGGATAA
AGTTGCAGGACCACTTCTGCGCTCGGCCCTTCCGGCTGGCTGGTTTATTGCTGATAAATCTGGAGCCGGTG
AGCGTGGGTCTCGCGGTATCATTGCAGCACTGGGGCCAGATGGTAAGCCCTCCCGTATCGTAGTTATCTAC
ACGACGGGGAGTCAGGCAACTATGGATGAACGAAATAGACAGATCGCTGAGATAGGTGCCTCACTGATT
AAGCATTGGTAACTGTCAGACCAAGTTTACTCATATATACTTTAGATTGATTTAAAACTTCATTTTTAATTT
AAAAGGATCTAGGTGAAGATCCTTTTTGATAATCTCATGACCAAAATCCCTTAACGTGAGTTTTCGTTCCA
CTGAGCGTCAGACCCCGTAGAAAAGATCAAAGGATCTTCTTGAGATCCTTTTTTTCTGCGCGTAATCTGCT
GCTTGCAAACAAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTTCTTCTAGTGTAGCCGTAGTTAGGCC
ACCACTTCAAGAACTCTGTAGCACCGCCTACATACCTCGCTCTGCTAATCCTGTTACCAGTGGCTGCTGCC
AGTGGCGATAAGTCGTGTCTTACCGGGTTGGACTCAAGACGATAGTTACCGGATAAGGCGCAGCGGTCGG
GCTGAACGGGGGGTTCGTGCACACAGCCCAGCTTGGAGCGAACGACCTACACCGAACTGAGATACCTAC
AGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGAGCCTATGGAAAAA
CGCCAGCAACGCGGCCTTTTTACGGTTCCTGGCCTTTTGCTGGCCTTTTGCTCACATGGCTCGACAGATCT
>SEQ ID NO: 38 RG6 PLASMID (HTTPS://WWW.ADDGENE.ORG/80167/)
GACGGATCGGGAGATCTCCCGATCCCCTATGGTCGACTCTCAGTACAATCTGCTCTGATGCCGCATAGTTA
AGCCAGTATCTGCTCCCTGCTTGTGTGTTGGAGGTCGCTGAGTAGTGCGCGAGCAAAATTTAAGCTACAA
CAAGGCAAGGCTTGACCGACAATTGCATGAAGAATCTGCTTAGGGTTAGGCGTTTTGCGCTGCTTCGCGA
TGTACGGGCCAGATATACGCGTTGACATTGATTATTGACTAGTTATTAATAGTAATCAATTACGGGGTCAT
TAGTTCATAGCCCATATATGGAGTTCCGCGTTACATAACTTACGGTAAATGGCCCGCCTGGCTGACCGCCC
AACGACCCCCGCCCATTGACGTCAATAATGACGTATGTTCCCATAGTAACGCCAATAGGGACTTTCCATTG
ACGTCAATGGGTGGACTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTACATGACCTTATGGG
ACTTTCCTACTTGGCAGTACATCTACGTATTAGTCATCGCTATTACCATGGTGATGCGGTTTTGGCAGTAC
ATCAATGGGCGTGGATAGCGGTTTGACTCACGGGGATTTCCAAGTCTCCACCCCATTGACGTCAATGGGA
GTTTGTTTTGGCACCAAAATCAACGGGACTTTCCAAAATGTCGTAACAACTCCGCCCCATTGACGCAAATG
GGCGGTAGGCGTGTACGGTGGGAGGTCTATATAAGCAGAGCTCTCTGGCTAACTAGAGAACCCACTGCTT
ACTGGCTTATCGAAATTAATACGACTCACTATAGGGAGACCCAAGCTGGCTAGCGTTTAAACTTAAGCTT
CCATGGATTACAAGGATGACGATGACAAGGGGGTACCTGCCCCAAAAAAAAAACGCAAAGTGGAGGACC
CAGTACCAGGATCTAGAGGTAGGTGATCCTCCTGCTGCTTTGGTTCAGGGTTTTGCTTGAGGGGGGGGGG
TGGTGATTTCCTTGCCATGGGCAGACTGAGCAGAAAAGGCCATTGGGACCATGTTCTGAATGCCTCCACC
TCAACCACCGGCCGGTAGGACCAAAGCCACCCCGTGTTTTCTCAGGATCTCTTTTCCCAGGGAGATCCCTC
GGCCCAAAGAGGGAGATGGCAATGCTGGATGTGTGCACAATAATTCAACAGGCATTGGAACTTCAGCATC
GATGCTGAATGCAATTAACAATGCTCAAGCAGAACCCCCGGCTCCATCAGCACAGTGCAGGACCAAACCC
CATGCTGCAGCAGTGGGGCTGTCTGTACGGGGTGGGCAATGGGAACCGGGGTCTGCTGGGGCTCCTGCTG
CTTCAGTGCTGCCATGCAGCCACACATCCTGAGAGCTGAAAGGGTCGGCGTCCTCACCTGGTGCACACCG
TAGCTCTGCCCCACAGCTTTAAGGCACCTGGCTAACCTCTGCGCTTCTTCCCTTCCCTCCTCCCTGGCTCAG
GATCCAGGCGATATCCGGAAGAATTCAGGTAGTTACTGCACCTTTCTTTGTTCCATCTCTCCACCTCTGCT
GTGAATAAATCGCGGGTCGGTGTGTCCTGTGCCTTTCCCTGCTTGGGAAACGCTTTCCTTTCATTCTTTCAC
TTCTCTGCTGCTTTTTGCGCTCTCCCCATCCTGCTGTGCCAACCTGCTCTCAGTTCTGTGCTTTCTGTCTTCC
ATCCCAACACACCCCTGGGTTGCTGTCTTCTTTCTCCTTTCTTCCTCTCTTGCTGTGGGACCAAACGTCTCC
TGCAGGACCTGCGGGCTCTGACAGAGGACTCTCGTGGGGGTACTGCTCCCTCCAGTGGAAAAATGCTCCA
GCAGTGTCATGCAGGAGATTTATGCCATACAGTTTTGCTCTCTGCTGCATGGAGGGGAGCAGCAGAAGTC
GATCTCCCCCACTCTGGGGTCCCCCTCGAGGGGGGCACAGCTGGGGAGGGAACAAGGGACAAAACCAGG
AGGGGGCTCCGAGTCCTTGGATTTATTCCCCCTCATCCATGCCTTACCTTCAGGTAAGGGCCTGAACAGAG
CCCTTTACTTCCTGCTTCTTTCTCCCATAGCTCCCTCTCCTTCGGGTCTCCTGGACTCAGTGCCACGGTTGTC
CCATTCTGGGGGTCTGTAGGGAGCCAGCAGGAGCTGCGGCCGTCCTACTGACCCTGTCCTTATTGCACAG
GTCAGGAGGATCAGGAGGACGAGGAGGAAGAGGAGACCGGTGTGCGCTCCTCCAAGAACGTCATCAAGG
AGTTCATGCGCTTCAAGGTGCGCATGGAGGGCACCGTGAACGGCCACGAGTTCGAGATCGAGGGCGAGG
GCGAGGGCCGCCCCTACGAGGGCCACAACACCGTGAAGCTGAAGGTGACCAAGGGCGGCCCCCTGCCCT
TCGCCTGGGACATCCTGTCCCCCCAGTTCCAGTACGGCTCCAAGGTGTACGTGAAGCACCCCGCCGACAT
CCCCGACTACAAGAAGCTGTCCTTCCCCGAGGGCTTCAAGTGGGAGCGCGTGATGAACTTCGAGGACGGC
GGCGTGGTGACCGTGACCCAGGACTCCTCCCTGCAGGACGGCTGCTTCATCTACAAGGTGAAGTTCATCG
GCGTGAACTTCCCCTCCGACGGCCCCGTAATGCAGAAGAAGACCATGGGCTGGGAGGCCTCCACCGAGCG
CCTGTACCCCCGCGACGGCGTGCTGAAGGGCGAGATCCACAAGGCCCTGAAGCTGAAGGACGGCGGCCA
CTACCTGGTGGAGTTCAAGTCCATCTACATGGCCAAGAAGCCCGTGCAGCTGCCCGGCTACTACTACGTG
GACTCCAAGCTGGACATCACCTCCCACAACGAGGACTACACCATCGTGGAGCAGTACGAGCGCACCGAG
GGCCGCCACCACCTGTTCCTGTAGACCGCGGTGTGAGCAAGGGCGAGGAGCTGTTCACCGGGGTGGTGCC
CATCCTGGTCGAGCTGGACGGCGACGTAAACGGCCACAAGTTCAGCGTGTCCGGCGAGGGCGAGGGCGA
TGCCACCTACGGCAAGCTGACCCTGAAGTTCATCTGCACCACCGGCAAGCTGCCCGTGCCCTGGCCCACC
CTCGTGACCACCCTGACCTACGGCGTGCAGTGCTTCAGCCGCTACCCCGACCACATGAAGCAGCACGACT
TCTTCAAGTCCGCCATGCCCGAAGGCTACGTCCAGGAGCGCACCATCTTCTTCAAGGACGACGGCAACTA
CAAGACCCGCGCCGAGGTGAAGTTCGAGGGCGACACCCTGGTGAACCGCATCGAGCTGAAGGGCATCGA
CTTCAAGGAGGACGGCAACATCCTGGGGCACAAGCTGGAGTACAACTACAACAGCCACAACGTCTATATC
ATGGCCGACAAGCAGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTGCTGCCCGACAACC
ACTACCTGAGCACCCAGTCCGCCCTGAGCAAAGACCCCAACGAGAAGCGCGATCACATGGTCCTGCTGGA
GTTCGTGACCGCCGCCGGGATCACTCTCGGCATGGACGAGCTGTACAAGTAAGGGCCCGTTTAAACCCGC
TGATCAGCCTCGACTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGAAGACAATAGCAGGCA
TGCTGGGGATGCGGTGGGCTCTATGGCTTCTGAGGCGGAAAGAACCAGCTGGGGCTCTAGGGGGTATCCC
CACGCGCCCTGTAGCGGCGCATTAAGCGCGGCGGGTGTGGTGGTTACGCGCAGCGTGACCGCTACACTTG
CCAGCGCCCTAGCGCCCGCTCCTTTCGCTTTCTTCCCTTCCTTTCTCGCCACGTTCGCCGGCTTTCCCCGTC
AAGCTCTAAATCGGGGCATCCCTTTAGGGTTCCGATTTAGTGCTTTACGGCACCTCGACCCCAAAAAACTT
GATTAGGGTGATGGTTCACGTAGTGGGCCATCGCCCTGATAGACGGTTTTTCGCCCTTTGACGTTGGAGTC
CACGTTCTTTAATAGTGGACTCTTGTTCCAAACTGGAACAACACTCAACCCTATCTCGGTCTATTCTTTTGA
TTTATAAGGGATTTTGGGGATTTCGGCCTATTGGTTAAAAAATGAGCTGATTTAACAAAAATTTAACGCGA
ATTAATTCTGTGGAATGTGTGTCAGTTAGGGTGTGGAAAGTCCCCAGGCTCCCCAGGCAGGCAGAAGTAT
GCAAAGCATGCATCTCAATTAGTCAGCAACCAGGTGTGGAAAGTCCCCAGGCTCCCCAGCAGGCAGAAGT
ATGCAAAGCATGCATCTCAATTAGTCAGCAACCATAGTCCCGCCCCTAACTCCGCCCATCCCGCCCCTAAC
TCCGCCCAGTTCCGCCCATTCTCCGCCCCATGGCTGACTAATTTTTTTTATTTATGCAGAGGCCGAGGCCG
CCTCTGCCTCTGAGCTATTCCAGAAGTAGTGAGGAGGCTTTTTTGGAGGCCTAGGCTTTTGCAAAAAGCTC
CCGGGAGCTTGTATATCCATTTTCGGATCTGATCAAGAGACAGGATGAGGATCGTTTCGCATGATTGAAC
AAGATGGATTGCACGCAGGTTCTCCGGCCGCTTGGGTGGAGAGGCTATTCGGCTATGACTGGGCACAACA
GACAATCGGCTGCTCTGATGCCGCCGTGTTCCGGCTGTCAGCGCAGGGGCGCCCGGTTCTTTTTGTCAAGA
CCGACCTGTCCGGTGCCCTGAATGAACTGCAGGACGAGGCAGCGCGGCTATCGTGGCTGGCCACGACGGG
CGTTCCTTGCGCAGCTGTGCTCGACGTTGTCACTGAAGCGGGAAGGGACTGGCTGCTATTGGGCGAAGTG
CCGGGGCAGGATCTCCTGTCATCTCACCTTGCTCCTGCCGAGAAAGTATCCATCATGGCTGATGCAATGCG
GCGGCTGCATACGCTTGATCCGGCTACCTGCCCATTCGACCACCAAGCGAAACATCGCATCGAGCGAGCA
CGTACTCGGATGGAAGCCGGTCTTGTCGATCAGGATGATCTGGACGAAGAGCATCAGGGGCTCGCGCCAG
CCGAACTGTTCGCCAGGCTCAAGGCGCGCATGCCCGACGGCGAGGATCTCGTCGTGACCCATGGCGATGC
CTGCTTGCCGAATATCATGGTGGAAAATGGCCGCTTTTCTGGATTCATCGACTGTGGCCGGCTGGGTGTGG
CGGACCGCTATCAGGACATAGCGTTGGCTACCCGTGATATTGCTGAAGAGCTTGGCGGCGAATGGGCTGA
CCGCTTCCTCGTGCTTTACGGTATCGCCGCTCCCGATTCGCAGCGCATCGCCTTCTATCGCCTTCTTGACGA
GTTCTTCTGAGCGGGACTCTGGGGTTCGAAATGACCGACCAAGCGACGCCCAACCTGCCATCACGAGATT
TCGATTCCACCGCCGCCTTCTATGAAAGGTTGGGCTTCGGAATCGTTTTCCGGGACGCCGGCTGGATGATC
CTCCAGCGCGGGGATCTCATGCTGGAGTTCTTCGCCCACCCCAACTTGTTTATTGCAGCTTATAATGGTTA
CAAATAAAGCAATAGCATCACAAATTTCACAAATAAAGCATTTTTTTCACTGCATTCTAGTTGTGGTTTGT
CCAAACTCATCAATGTATCTTATCATGTCTGTATACCGTCGACCTCTAGCTAGAGCTTGGCGTAATCATGG
TCATAGCTGTTTCCTGTGTGAAATTGTTATCCGCTCACAATTCCACACAACATACGAGCCGGAAGCATAAA
GTGTAAAGCCTGGGGTGCCTAATGAGTGAGCTAACTCACATTAATTGCGTTGCGCTCACTGCCCGCTTTCC
AGTCGGGAAACCTGTCGTGCCAGCTGCATTAATGAATCGGCCAACGCGCGGGGAGAGGCGGTTTGCGTAT
TGGGCGCTCTTCCGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGTGAGCAA
AAGGCCAGCAAAAGGCCAGGAACCGTAAAAAGGCCGCGTTGCTGGCGTTTTTCCATAGGCTCCGCCCCCC
TGACGAGCATCACAAAAATCGACGCTCAAGTCAGAGGTGGCGAAACCCGACAGGACTATAAAGATACCA
GGCGTTTCCCCCTGGAAGCTCCCTCGTGCGCTCTCCTGTTCCGACCCTGCCGCTTACCGGATACCTGTCCG
CCTTTCTCCCTTCGGGAAGCGTGGCGCTTTCTCAATGCTCACGCTGTAGGTATCTCAGTTCGGTGTAGGTC
GTTCGCTCCAAGCTGGGCTGTGTGCACGAACCCCCCGTTCAGCCCGACCGCTGCGCCTTATCCGGTAACTA
TCGTCTTGAGTCCAACCCGGTAAGACACGACTTATCGCCACTGGCAGCAGCCACTGGTAACAGGATTAGC
AGAGCGAGGTATGTAGGCGGTGCTACAGAGTTCTTGAAGTGGTGGCCTAACTACGGCTACACTAGAAGGA
CAGTATTTGGTATCTGCGCTCTGCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCAGTGGAACGAAAACTCACGTTAAGGGATT
TTGGTCATGAGATTATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGCACCTATCTCAGCG
ATCTGTCTATTTCGTTCATCCATAGTTGCCTGACTCCCCGTCGTGTAGATAACTACGATACGGGAGGGCTT
ACCATCTGGCCCCAGTGCTGCAATGATACCGCGAGACCCACGCTCACCGGCTCCAGATTTATCAGCAATA
AACCAGCCAGCCGGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTTGCCATTGCTACA
GGCATCGTGGTGTCACGCTCGTCGTTTGGTATGGCTTCATTCAGCTCCGGTTCCCAACGATCAAGGCGAGT
TACATGATCCCCCATGTTGTGCAAAAAAGCGGTTAGCTCCTTCGGTCCTCCGATCGTTGTCAGAAGTAAGT
TGGCCGCAGTGTTATCACTCATGGTTATGGCAGCACTGCATAATTCTCTTACTGTCATGCCATCCGTAAGA
TGCTTTTCTGTGACTGGTGAGTACTCAACCAAGTCATTCTGAGAATAGTGTATGCGGCGACCGAGTTGCTC
TTGCCCGGCGTCAATACGGGATAATACCGCGCCACATAGCAGAACTTTAAAAGTGCTCATCATTGGAAAA
CGTTCTTCGGGGCGAAAACTCTCAAGGATCTTACCGCTGTTGAGATCCAGTTCGATGTAACCCACTCGTGC
ACCCAACTGATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCAATATTATT
GAAGCATTTATCAGGGTTATTGTCTCATGAGCGGATACATATTTGAATGTATTTAGAAAAATAAACAAAT
AGGGGTTCCGCGCACATTTCCCCGAAAAGTGCCACCTGACGTC
>SEQ ID NO: 39 PCI-SMN2-F
GCTAACGCAGTCAGTGCTTC
>SEQ ID NO: 40 PCI-SMN2-R
GTATCTTATCATGTCTGCTCG
>SEQ ID NO: 41 RG6-F
ATGGATTACAAGGATGACGATGAC
>SEQ ID NO: 42 RG6-R
GCGCATGAACTCCTTGATGAC
>SEQ ID NO: 43 split N652-CASFx
MNCEREQLRGNQEAAAAPDTMAQPYASAQFAPPQNGIPAEYTAPHPHPAPEYTGQTTVPEHTLNLYPPAQTHS
EQSPADTSAQTVSGTATQTDDAAPTDGQPQTQPSENTENKSQPKGGGGSGRASPKKKRKVEASIEKKKSFAKG
MGVKSTLVSGSKVYMTTFAEGSDARLEKIVEGDSIRSVNEGEAFSAEMADKNAGYKIGNAKFSHPKGYAVVA
NNPLYTGPVQQDMLGLKETLEKRYFGESADGNDNICIQVIHNILDIEKILAEYITNAAYAVNNISGLDKDIIGFG
KFSTVYTYDEFKDPEHHRAAFNNNDKLINAIKAQYDEFDNFLDNPRLGYFGQAFFSKEGRNYIINYGNECYDIL
ALLSGLAHWVVANNEEESRISRTWLYNLDKNLDNEYISTLNYLYDRITNELTNSFSKNSAANVNYIAETLGINP
AEFAEQYFRFSIMKEQKNLGFNITKLREVMLDRKDMSEIRKNHKVFDSIRTKVYTMMDFVIYRYYIEEDAKVA
AANKSLPDNEKSLSEKDIFVINLRGSFNDDQKDALYYDEANRIWRKLENIMHNIKEFRGNKTREYKKKDAPRL
PRILPAGRDVSAFSKLMYALTMFLDGKEINDLLTTLINKFDNIQSFLKVMPLIGVNAKFVEEYAFFKDSAKIADE
LRLIKSFARMGEPIADARRAMYIDAIRILGTNLSYDELKALADTFSLDENGNKLKKGKHGMRNFIINNVISNKRF
HYLIRYGDPAHLHEIAKNEAVVKFVLGRIADIQKKQGQNGKNQIDRYYETCLSYETEILTVEYGLLPIGKIVEKR
IECTVYSVDNNGNIYTQPVAQWHDRGEQEVFEYCLEDGSLIRATKDHKFMTVDGQMLPIDEIFERELDLMRVD
NLPN
>SEQ ID NO: 44 split C654-CASFx
MIKIATRKYLGKQNVYDIGVERDHNFALKNGFIASNCIGKDKGKSVSEKVDALTKIITGMNYDQFDKKRSVIE
DTGRENAEREKFKKIISLYLTVIYHILKNIVNINARYVIGFHCVERDAQLYKEKGYDINLKKLEEKGFSSVTKLC
AGIDETAPDKRKDVEKEMAERAKESIDSLESANPKLYANYIKYSDEKKAEEFTRQINREKAKTALNAYLRNTK
WNVIIREDLLRIDNKTCTLFANKAVALEVARYVHAYINDIAEVNSYFQLYHYIMQRIIMNERYEKSSGKVSEYF
DAVNDEKKYNDRLLKLLCVPFGYCIPRFKNLSIEALFDRNEAAKFDKEKKKVSGNSGSGPKKKRKVAAAYPY
DVPDYAGGRGGGGSGGGGSGGGGSGPANATARVMTNKKTVNPYTNGWKLNPVVGAVYSPEFYAGTVLLCQ
ANQEGSSMYSAPSSLVYTSAMPGFPYPAATAAAAYRGAHLRGRGRTVYNTFRAAAPPPPIPAYGGVVYQDGF
YGADIYGGYAAYRYAQPTPATAAAYSDSYGRVYAADPYHHALAPAPTYGVGAMNAFAPLTDAKTRSHADD
VGLVLSSLQASIYRGGYNRFAPY
>SEQ ID NO: 45 split N463-CASFx
MNCEREQLRGNQEAAAAPDTMAQPYASAQFAPPQNGIPAEYTAPHPHPAPEYTGQTTVPEHTLNLYPPAQTHS
EQSPADTSAQTVSGTATQTDDAAPTDGQPQTQPSENTENKSQPKGGGGSGRASPKKKRKVEASIEKKKSFAKG
MGVKSTLVSGSKVYMTTFAEGSDARLEKIVEGDSIRSVNEGEAFSAEMADKNAGYKIGNAKFSHPKGYAVVA
NNPLYTGPVQQDMLGLKETLEKRYFGESADGNDNICIQVIHNILDIEKILAEYITNAAYAVNNISGLDKDIIGFG
KFSTVYTYDEFKDPEHHRAAFNNNDKLINAIKAQYDEFDNFLDNPRLGYFGQAFFSKEGRNYIINYGNECYDIL
ALLSGLAHWVVANNEEESRISRTWLYNLDKNLDNEYISTLNYLYDRITNELTNSFSKNSAANVNYIAETLGINP
AEFAEQYFRFSIMKEQKNLGFNITKLREVMLDRKDMSEIRKNHKVFDSIRTKVYTMMDFVIYRYYIEEDAKVA
AANKSLPDNEKSLSEKDIFVINLRGSFNDDQKDALYYDEANRIWRKLENIMHNIKEFRGNKTREYKKKDAPRL
PRILPAGRDVSCLSYETEILTVEYGLLPIGKIVEKRIECTVYSVDNNGNIYTQPVAQWHDRGEQEVFEYCLEDGS
LIRATKDHKFMTVDGQMLPIDEIFERELDLMRVDNLPN
>SEQ ID NO: 46 split C+30C464-CASFx
MIKIATRKYLGKQNVYDIGVERDHNFALKNGFIASNCAFSKLMYALTMFLDGKEINDLLTTLINKFDNIQSFLK
VMPLIGVNAKFVEEYAFFKDSAKIADELRLIKSFARMGEPIADARRAMYIDAIRILGTNLSYDELKALADTFSLD
ENGNKLKKGKHGMRNFIINNVISNKRFHYLIRYGDPAHLHEIAKNEAVVKFVLGRIADIQKKQGQNGKNQIDR
YYETCIGKDKGKSVSEKVDALTKIITGMNYDQFDKKRSVIEDTGRENAEREKFKKIISLYLTVIYHILKNIVNIN
ARYVIGFHCVERDAQLYKEKGYDINLKKLEEKGFSSVTKLCAGIDETAPDKRKDVEKEMAERAKESIDSLESA
NPKLYANYIKYSDEKKAEEFTRQINREKAKTALNAYLRNTKWNVIIREDLLRIDNKTCTLFANKAVALEVARY
VHAYINDIAEVNSYFQLYHYIMQRIIMNERYEKSSGKVSEYFDAVNDEKKYNDRLLKLLCVPFGYCIPRFKNLS
IEALFDRNEAAKFDKEKKKVSGNSGSGPKKKRKVAAAYPYDVPDYAGGRGGGGSGGGGSGGGGSGPANATA
RVMTNKKTVNPYTNGWKLNPVVGAVYSPEFYAGTVLLCQANQEGSSMYSAPSSLVYTSAMPGFPYPAATAA
AAYRGAHLRGRGRTVYNTFRAAAPPPPIPAYGGVVYQDGFYGADIYGGYAAYRYAQPTPATAAAYSDSYGR
VYAADPYHHALAPAPTYGVGAMNAFAPLTDAKTRSHADDVGLVLSSLQASIYRGGYNRFAPY
>SEQ ID NO: 47 split N497-CASFx
MNCEREQLRGNQEAAAAPDTMAQPYASAQFAPPQNGIPAEYTAPHPHPAPEYTGQTTVPEHTLNLYPPAQTHS
EQSPADTSAQTVSGTATQTDDAAPTDGQPQTQPSENTENKSQPKGGGGSGRASPKKKRKVEASIEKKKSFAKG
MGVKSTLVSGSKVYMTTFAEGSDARLEKIVEGDSIRSVNEGEAFSAEMADKNAGYKIGNAKFSHPKGYAVVA
NNPLYTGPVQQDMLGLKETLEKRYFGESADGNDNICIQVIHNILDIEKILAEYITNAAYAVNNISGLDKDIIGFG
KFSTVYTYDEFKDPEHHRAAFNNNDKLINAIKAQYDEFDNFLDNPRLGYFGQAFFSKEGRNYIINYGNECYDIL
ALLSGLAHWVVANNEEESRISRTWLYNLDKNLDNEYISTLNYLYDRITNELTNSFSKNSAANVNYIAETLGINP
AEFAEQYFRFSIMKEQKNLGFNITKLREVMLDRKDMSEIRKNHKVFDSIRTKVYTMMDFVIYRYYIEEDAKVA
AANKSLPDNEKSLSEKDIFVINLRGSFNDDQKDALYYDEANRIWRKLENIMHNIKEFRGNKTREYKKKDAPRL
PRILPAGRDVSAFSKLMYALTMFLDGKEINDLLTTLINKFDNIQSCLSYETEILTVEYGLLPIGKIVEKRIECTVYS
VDNNGNIYTQPVAQWHDRGEQEVFEYCLEDGSLIRATKDHKFMTVDGQMLPIDEIFERELDLMRVDNLPN
>SEQ ID NO: 48 split C + C498-CASFx
MIKIATRKYLGKQNVYDIGVERDHNFALKNGFIASNCFLKVMPLIGVNAKFVEEYAFFKDSAKIADELRLIKSF
ARMGEPIADARRAMYIDAIRILGTNLSYDELKALADTFSLDENGNKLKKGKHGMRNFIINNVISNKRFHYLIRY
GDPAHLHEIAKNEAVVKFVLGRIADIQKKQGQNGKNQIDRYYETCIGKDKGKSVSEKVDALTKIITGMNYDQF
DKKRSVIEDTGRENAEREKFKKIISLYLTVIYHILKNIVNINARYVIGFHCVERDAQLYKEKGYDINLKKLEEKG
FSSVTKLCAGIDETAPDKRKDVEKEMAERAKESIDSLESANPKLYANYIKYSDEKKAEEFTRQINREKAKTALN
AYLRNTKWNVIIREDLLRIDNKTCTLFANKAVALEVARYVHAYINDIAEVNSYFQLYHYIMQRIIMNERYEKSS
GKVSEYFDAVNDEKKYNDRLLKLLCVPFGYCIPRFKNLSIEALFDRNEAAKFDKEKKKVSGNSGSGPKKKRKV
AAAYPYDVPDYAGGRGGGGSGGGGSGGGGSGPANATARVMTNKKTVNPYTNGWKLNPVVGAVYSPEFYA
GTVLLCQANQEGSSMYSAPSSLVYTSAMPGFPYPAATAAAAYRGAHLRGRGRTVYNTFRAAAPPPPIPAYGG
VVYQDGFYGADIYGGYAAYRYAQPTPATAAAYSDSYGRVYAADPYHHALAPAPTYGVGAMNAFAPLTDAK
TRSHADDVGLVLSSLQASIYRGGYNRFAPY
>SEQ ID NO: 49 SNRPC-dCasRx
MPKFYCDYCDTYLTHDSPSVRKTHCSGRKHKENVKDYYQKWMEEQAQSLIDKTTAAFQQGKIPPTPFSAPPP
AGAMIPPPPSLPGPPRPGMMPAPHMGGPPMMPMMGPPPPGMMPVGPAPGMRPPMGGHMPMMPGPPMMRPP
ARPMMVPTRPGMTRPDRNVIDGGGGSDPKKKRKVDPKKKRKVDPKKKRKVGSTGSRNDGGGGSGGGGSGG
GGSGRASPKKKRKVEASIEKKKSFAKGMGVKSTLVSGSKVYMTTFAEGSDARLEKIVEGDSIRSVNEGEAFSA
EMADKNAGYKIGNAKFSHPKGYAVVANNPLYTGPVQQDMLGLKETLEKRYFGESADGNDNICIQVIHNILDIE
KILAEYITNAAYAVNNISGLDKDIIGFGKFSTVYTYDEFKDPEHHRAAFNNNDKLINAIKAQYDEFDNFLDNPR
LGYFGQAFFSKEGRNYIINYGNECYDILALLSGLAHWVVANNEEESRISRTWLYNLDKNLDNEYISTLNYLYD
RITNELTNSFSKNSAANVNYIAETLGINPAEFAEQYFRFSIMKEQKNLGFNITKLREVMLDRKDMSEIRKNHKV
FDSIRTKVYTMMDFVIYRYYIEEDAKVAAANKSLPDNEKSLSEKDIFVINLRGSFNDDQKDALYYDEANRIWR
KLENIMHNIKEFRGNKTREYKKKDAPRLPRILPAGRDVSAFSKLMYALTMFLDGKEINDLLTTLINKFDNIQSFL
KVMPLIGVNAKFVEEYAFFKDSAKIADELRLIKSFARMGEPIADARRAMYIDAIRILGTNLSYDELKALADTFSL
DENGNKLKKGKHGMRNFIINNVISNKRFHYLIRYGDPAHLHEIAKNEAVVKFVLGRIADIQKKQGQNGKNQID
RYYETCIGKDKGKSVSEKVDALTKIITGMNYDQFDKKRSVIEDTGRENAEREKFKKIISLYLTVIYHILKNIVNIN
ARYVIGFHCVERDAQLYKEKGYDINLKKLEEKGFSSVTKLCAGIDETAPDKRKDVEKEMAERAKESIDSLESA
NPKLYANYIKYSDEKKAEEFTRQINREKAKTALNAYLRNTKWNVIIREDLLRIDNKTCTLFANKAVALEVARY
VHAYINDIAEVNSYFQLYHYIMQRIIMNERYEKSSGKVSEYFDAVNDEKKYNDRLLKLLCVPFGYCIPRFKNLS
IEALFDRNEAAKFDKEKKKVSGNSGSGPKKKRKVAAAYPYDVPDYAGGRGGGGSGGGGSGGGGSGPAMDY
KDHDGDYKDHDIDYKDDDDK
>SEQ ID NO: 50 dNMCas9-RBM38
MDYKDHDGDYKDHDIDYKDDDDKIDGGGGSDPKKKRKVDPKKKRKVDPKKKRKVGSTGSRNDGGGGSGG
GGSGGGGSGRAAAFKPNPINYILGLAIGIASVGWAMVEIDEDENPICLIDLGVRVFERAEVPKTGDSLAMARRL
ARSVRRLTRRRAHRLLRARRLLKREGVLQAADFDENGLIKSLPNTPWQLRAAALDRKLTPLEWSAVLLHLIKH
RGYLSQRKNEGETADKELGALLKGVADNAHALQTGDFRTPAELALNKFEKESGHIRNQRGDYSHTFSRKDLQ
AELILLFEKQKEFGNPHVSGGLKEGIETLLMTQRPALSGDAVQKMLGHCTFEPAEPKAAKNTYTAERFIWLTK
LNNLRILEQGSERPLTDTERATLMDEPYRKSKLTYAQARKLLGLEDTAFFKGLRYGKDNAEASTLMEMKAYH
AISRALEKEGLKDKKSPLNLSPELQDEIGTAFSLFKTDEDITGRLKDRIQPEILEALLKHISFDKFVQISLKALRRI
VPLMEQGKRYDEACAEIYGDHYGKKNTEEKIYLPPIPADEIRNPVVLRALSQARKVINGVVRRYGSPARIHIET
AREVGKSFKDRKEIEKRQEENRKDREKAAAKFREYFPNFVGEPKSKDILKLRLYEQQHGKCLYSGKEINLGRL
NEKGYVEIAAALPFSRTWDDSFNNKVLVLGSEAQNKGNQTPYEYFNGKDNSREWQEFKARVETSRFPRSKKQ
RILLQKFDEDGFKERNLNDTRYVNRFLCQFVADRMRLTGKGKKRVFASNGQITNLLRGFWGLRKVRAENDRH
HALDAVVVACSTVAMQQKITRFVRYKEMNAFDGKTIDKETGEVLHQKTHFPQPWEFFAQEVMIRVFGKPDG
KPEFEEADTPEKLRTLLAEKLSSRPEAVHEYVTPLFVSRAPNRKMSGQGHMETVKSAKRLDEGVSVLRVPLTQ
LKLKDLEKMVNREREPKLYEALKARLEAHKDDPAKAFAEPFYKYDKAGNRTQQVKAVRVEQVQKTGVWVR
NHNGIADNATMVRVDVFEKGDKYYLVPIYSWQVAKGILPDRAVVQGKDEEDWQLIDDSFNFKFSLHPNDLVE
VITKKARMFGYFASCHRGTGNINIRIHDLDHKIGKNGILEGIGVKTALSFQKYQIDELGKEIRPCRLKKRPPVRG
STSGSPKKKRKVGGGRGGGGSGGGGSGGGGSGPAMLLQPAPCAPSAGFPRPLAAPGAMHGSQKDTTFTKIFV
GGLPYHTTDASLRKYFEGFGDIEEAVVITDRQTGKSRGYGFVTMADRAAAERACKDPNPIIDGRKANVNLAYL
GAKPRSLQTGFAIGVQQLHPTLIQRTYGLTPHYIYPPAIVQPSVVIPAAPVPSLSSPYIEYTPASPAYAQYPPATY
DQYPYAASPATAASFVGYSYPAAVPQALSAAAPAGTTFVQYQAPQLQPDRMQ
>SEQ ID NO: 51 NC (non-targeting control) gRNA
GATATCGCCTGGATCCTGAGCCAGGTTGTAGCTCCCTTTCTCATTTCGGAAACGAAATGAGAACCGTTGCT
ACAATAAGGCCGTCTGAAAAGATGTGCCGCAACGCTCTGCCCCTTAAAGCTTCTGCTTTAAGGGGCATCG
TTTAATTTTTTT
>SEQ ID NO: 52 N1 gRNA
GTTACAAAAGTAAGATTCACTTTCAGTTGTAGCTCCCTTTCTCATTTCGGAAACGAAATGAGAACCGTTGC
TACAATAAGGCCGTCTGAAAAGATGTGCCGCAACGCTCTGCCCCTTAAAGCTTCTGCTTTAAGGGGCATC
GTTTAATTTTTTT
>SEQ ID NO: 53 N2 gRNA
GAGAATTCTAGTAGGGATGTAGATGTTGTAGCTCCCTTTCTCATTTCGGAAACGAAATGAGAACCGTTGCT
ACAATAAGGCCGTCTGAAAAGATGTGCCGCAACGCTCTGCCCCTTAAAGCTTCTGCTTTAAGGGGCATCG
TTTAATTTTTTT
>SEQ ID NO: 54 N3 gRNA
GTTTCTTCCACACAACCAACCAGTGTTGTAGCTCCCTTTCTCATTTCGGAAACGAAATGAGAACCGTTGCT
ACAATAAGGCCGTCTGAAAAGATGTGCCGCAACGCTCTGCCCCTTAAAGCTTCTGCTTTAAGGGGCATCG
TTTAATTTTTTT
>SEQ ID NO: 55 Inclusion Isoform Forward Primer
ATAATTCCCCCACCACCTC
>SEQ ID NO: 56 Inclusion Isoform Reverse Primer
CTTCTTTTTGATTTTGTCTAAAACCCATATAATAG
>SEQ ID NO: 57 Exclusion Isoform Forward Primer
ATAATTCCCCCACCACCTC
>SEQ ID NO: 58 Exclusion Isoform Reverse Primer
CTCTATGCCAGCATTTCCATATAATAG

### EXAMPLES

### Example 1. An RNA-Guided Artificial Splicing Factor RBFOX1N-dCasRx-C Activates SMN2-E7

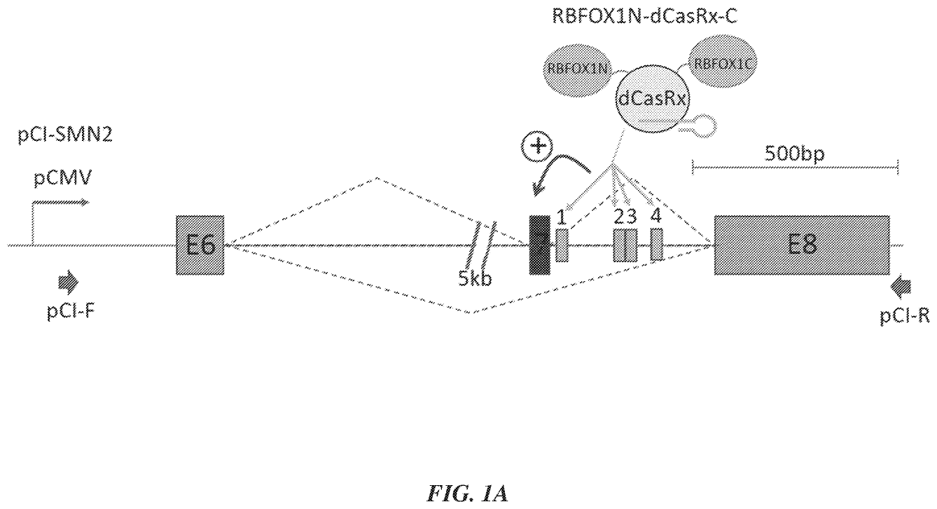

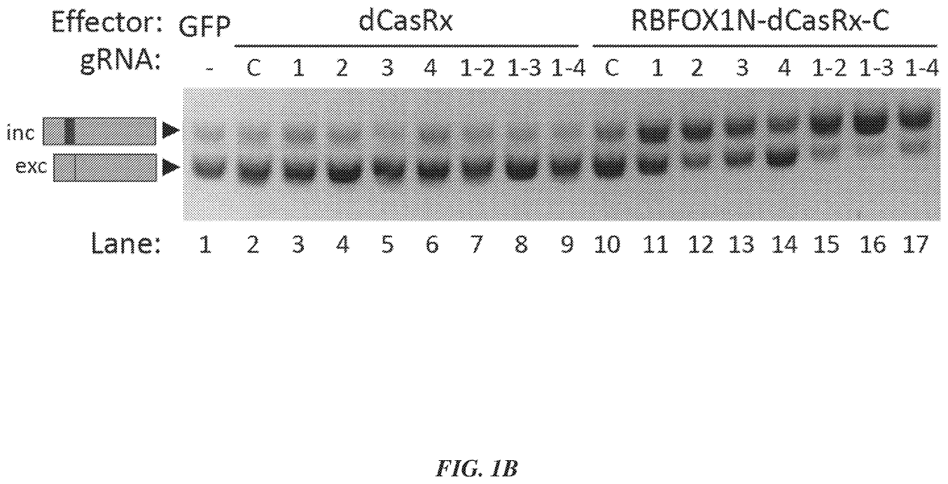

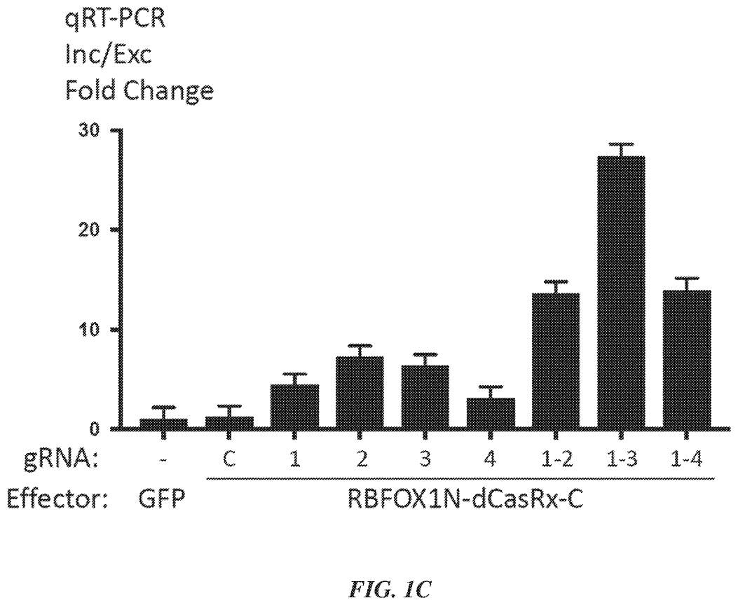

We created an artificial RNA-guided splicing factor (RBFOX1N-dCasRx-C) by replacing segments containing the RNA binding domain of splicing factor RBFOX1 (residues 118-189) with dCasRx and tested its activity to induce inclusion of Exon 7 of SMN2 (SMN2-E7) in the presence of targeting guide RNAs (gRNAs) (FIG. 1A). Four gRNAs (gSMN2-1 through gSMN2-4) were designed within the intron between SMN2-E7 and E8. When transfected with pCI-SMN2 and control GFP plasmid (pmaxGFP), SMN2 minigene expressed predominantly exclusion isoform (FIG. 1B, lane 1). When transfected with RBFOX1N-dCasRx-C and individual gRNAs, inclusion isoform level increased (FIG. 1B, lanes 11-14, see upper bands). Introduction of pools of two, three or four gRNAs simultaneously, increased further E7-included transcripts, as well as decreased the level of E7-excluded transcripts, switching the splicing pattern to predominantly inclusion (FIG. 1B, lanes 15-16). SMN2-E7 activation is dependent on RBFOX1 effector because dCasRx alone did not result in activation (FIG. 1B, lanes 2-9). Activation is also dependent on binding of the RBFOX1N-dCasRx-C on the SMN2 intron as control gRNAs (“C”) did not induce SMN2-E7 inclusion (FIG. 1B, lanes 2 and 10). To further quantitate the effect of SMN2-E7 activation, we conducted quantitative RT-PCR (qRT-PCR) using SYBR green reagents and primer pairs corresponding to E7-inclusion or E7-exclusion isoforms (FIG. 1C). We observed fold changes of inc/exc ratio compared to control GFP transfection consistent with the patterns observed in the semiquantitative RT-PCR assay, with pools of three gRNAs (gSMN2-1 through 3) giving the highest fold change.

### Example 2. RNA-Guided Artificial Splicing Factor RBM38-dCasRx and dCasRx-RBM38 Activates SMN2-E7

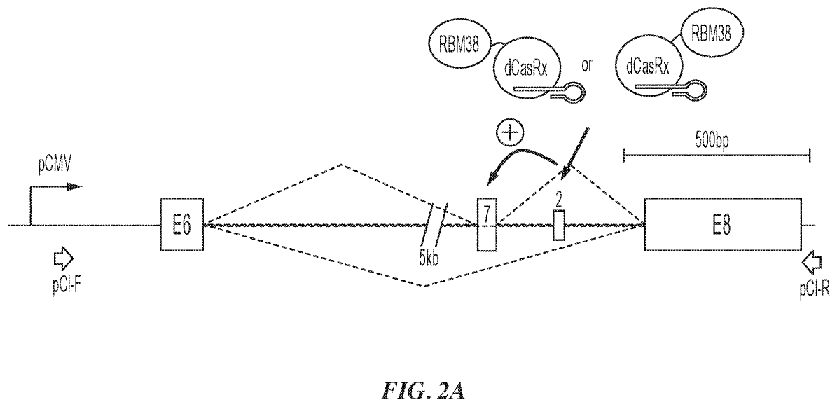

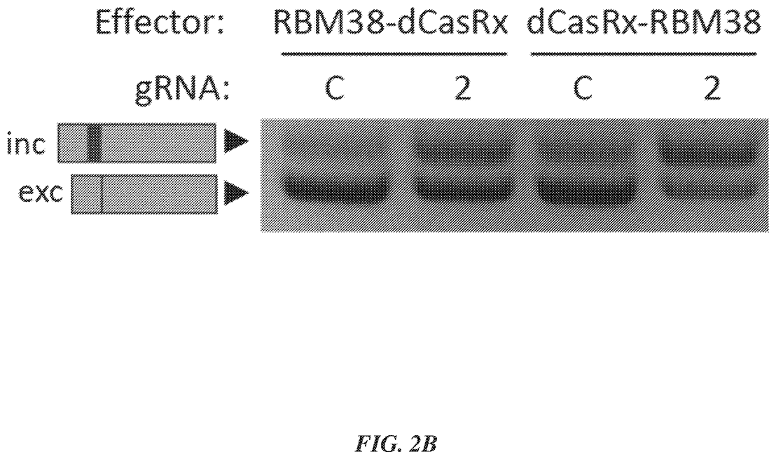

We constructed two other artificial splicing factors by fusing RBM38 to the N-terminus (RBM38-dCasRx) or C-terminus (dCasRx-RBM38) of dCasRx and tested its ability to active SMN2-E7 (FIG. 2A). By guiding the artificial splicing factors to intronic sequences between SMN2-E7 and E8, we observed increase in E7 inclusion, with a switch to E7-dominance observed for the dCasRx-RBM38 fusion configuration (FIG. 2B).

### Example 3. Both Exon Activation and Repression can be Effected by RBFOX1N-dCasRx-C, RBM38-dCasRx or dCasRx-RBM38 by Differential Positioning of Target Sites

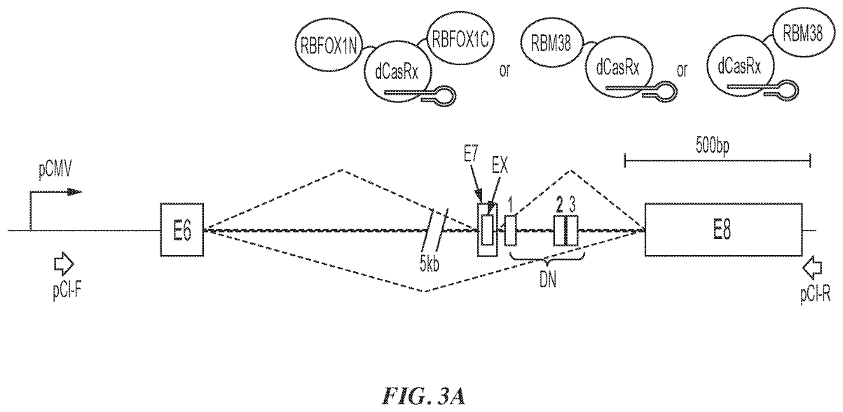

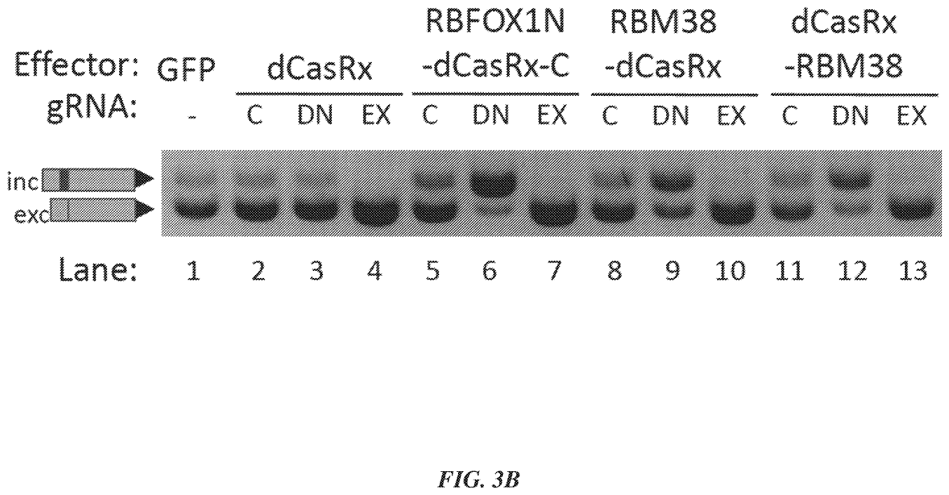

We investigated whether the RNA-guided artificial splicing activators can also induce exon skipping (exclusion) by binding to a different location (FIG. 3A). We designed a gRNA targeting within SMN2-E7 and found that it can direct RBFOX1N-dCasRx-C, RBM38-dCasRx or dCasRx-RBM38 to induce skipping of E7 (FIG. 3B, lanes 7,10,13). However, the splicing domains were not required for exon exclusion because unfused dCasRx was also capable of inducing exon skipping (FIG. 3B, lane 4). Nonetheless, the RNA-guided artificial splicing factors can induce both inclusion (FIG. 3B, lanes 6,9,12) or exclusion of exons (FIG. 3B, lanes 7,10,13) depending on the designed locations of targeting, providing a dual functionality for splicing modulation.

### Example 4. Simultaneous Activation and Repression of Two Independent Exons by RBFOX1N-dCasRx-C

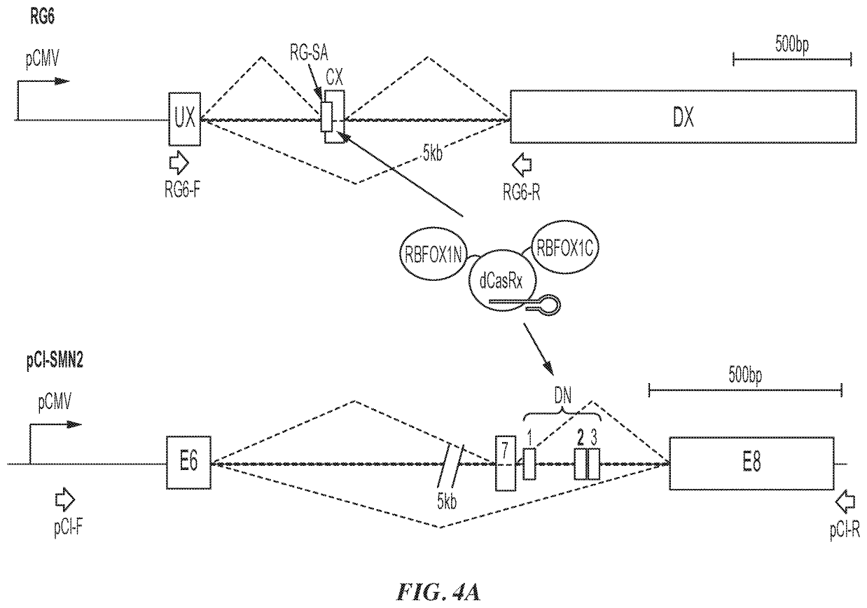

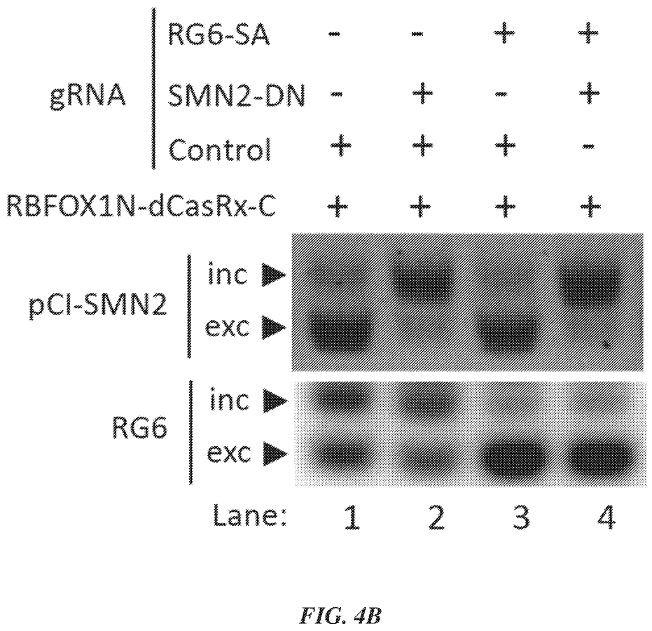

Given that we can activate or repress exons by differential positioning of targeting, we further tested whether we can exploit such property to simultaneously activate and repress two independent exons by RNA-guided artificial splicing factors. We simultaneously target RBFOX1N-dCasRx-C to splice acceptor (SA) site of RG6 minigene using gRNA RG6-SA, and sites downstream of SMN2-E7 of the SMN2 minigene using a pool of gRNAs (DN) (FIG. 4A). We observed simultaneous activation of SMN2-E7 and repression of RG6 cassette exon (CX) when both RG6-SA gRNA and DN gRNAs were co-transfected with RBFOX1N-dCasRx-C into cells (FIG. 4B, lane 4) compared to control (FIG. 4B, lane 1). These modulations are gRNA-dependent because when either of these gRNAs were replaced by Control gRNA (FIG. 4B, lanes 2 and 3), the splicing pattern of the corresponding target exon resemble the control cells (FIG. 4B, lane 1).

### Example 5. A Three-Component Two-Peptide Artificial Splicing Factor Activates SMN2-E7

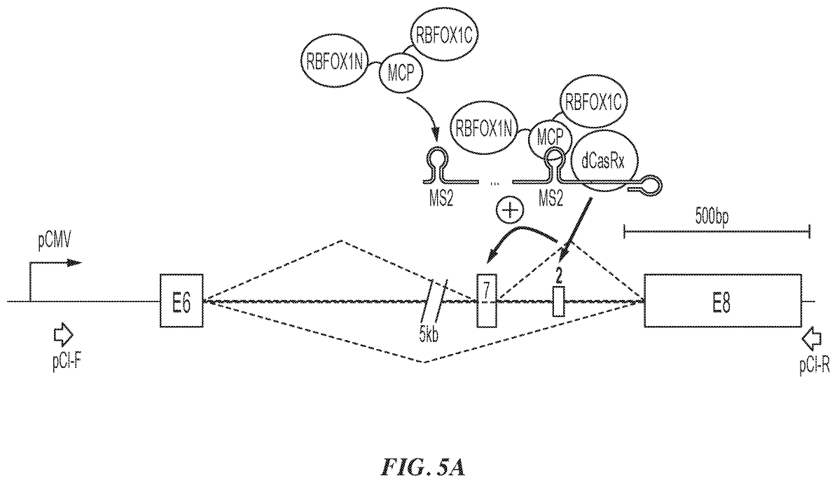

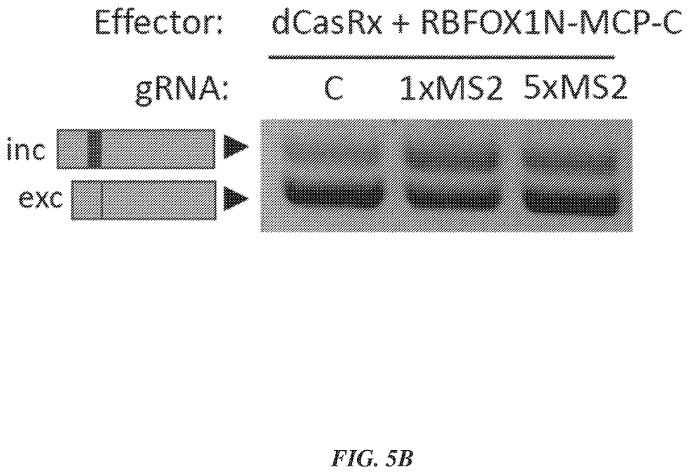

To allow for flexibility of targeting, we tested whether we could separate the effector function from the targeting domain of an artificial splicing factor into two separate peptides. Such design will allow dissociation of target recognition and effector operation that can be reconstituted by bridging gRNAs. The effector module is constructed by replacing RNA binding domain of RBFOX1 with MS2 coat protein (MCP), resulting in RBFOX1N-MCP-C (FIG. 5A). A modified gRNA with one or more copy of MS2 hairpins appended at the 3′ end guides dCasRx to the target RNA as well as recruits the effector module RBFOX1N-MCP-C via the MS2 hairpins. A functional splicing factor is thus assembled at the target. We observed increase of SMN2-E7 levels in cells transfected with this artificial splicing factor with SMN2 intron targeting gRNAs with 1 or 5 MS2 hairpins, demonstrating such strategy of constructing a three-component two-peptide artificial splicing factor worked (FIG. 5B).

### Example 6. Polycistronic Pre-gRNA Supports Multiplex Splicing Modulation

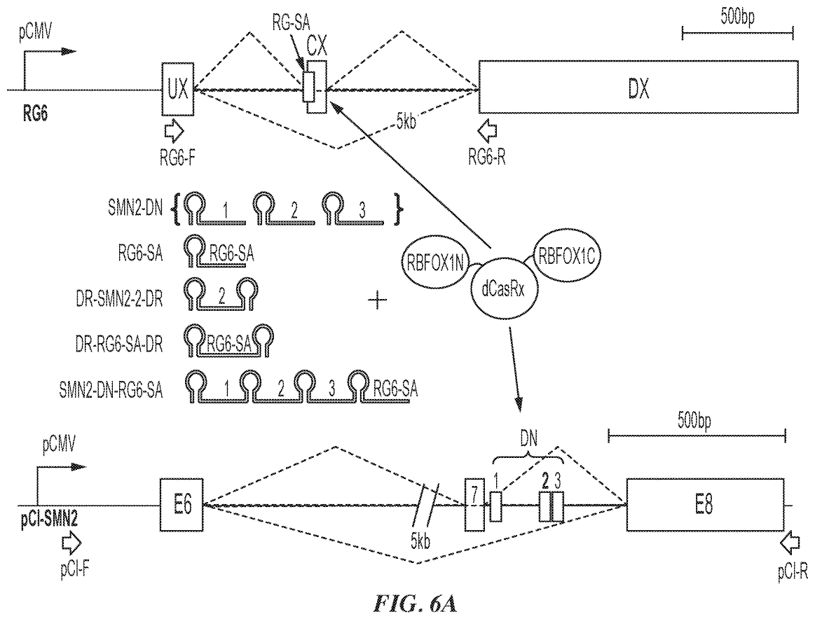

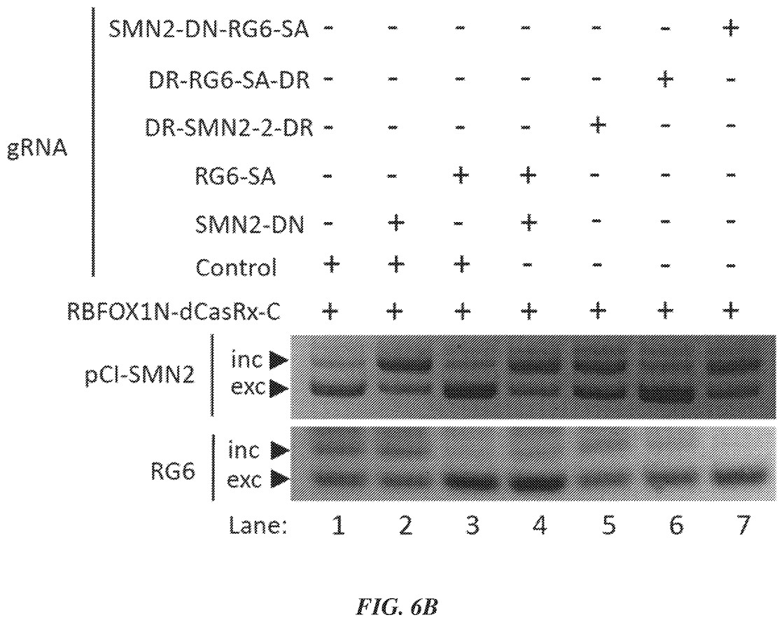

CasRx is capable of processing gRNAs encoded in tandem (pre-gRNA) by cleaving 5′ of the direct repeat (DR) stem loop structures. We tested whether we could make use of such property to encode gRNAs in tandem on one plasmid, and compare that with different gRNA architectures (FIG. 6A). As described in earlier examples in this application, we could induce simultaneous exon activation and skipping on SMN2 and RG6, respectively, when a mixture of plasmids each expressing one gRNA targeting these two splicing events were co-transfected in conjunction with RBFOX1N-dCasRx-C into cells (FIG. 6B, lane 4). We then tested whether gRNA with two DRs flanking targeting spacer could be processed by CasRx into functional mature gRNAs to affect splicing. As shown in FIG. 6B (lanes 5 and 6), double DR-flanked gRNAs DR-SMN2-2-DR, DR-RG6-SA-DR, containing spacers flanked by two direct repeats (DR), were able to direct RBFOX1N-dCasRx-C to induce exon inclusion and exclusion, respectively. We then tested the functionality of a polycistronic pre-gRNA (SMN2-DN-RG6-SA) containing three DN spacer targeting SMN2 intron and a splice acceptor spacer targeting RG6 cassette exon (RG6-CX) encoded in tandem and separated by DRs. As shown in FIG. 6B (lane 7), such pre-gRNA architecture enabled simultaneous inclusion of SMN2-E7 and exclusion of RG6-CX.

### Example 7. dCasRx-DAZAP1(191-407) Activates Splicing when Bound at Downstream Intron

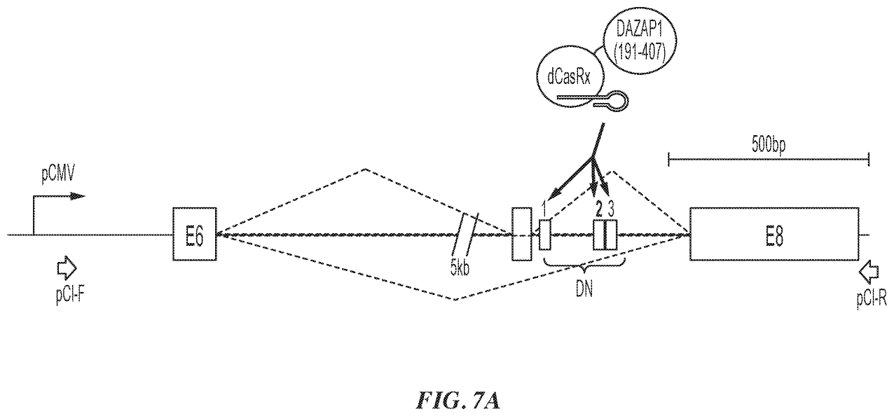

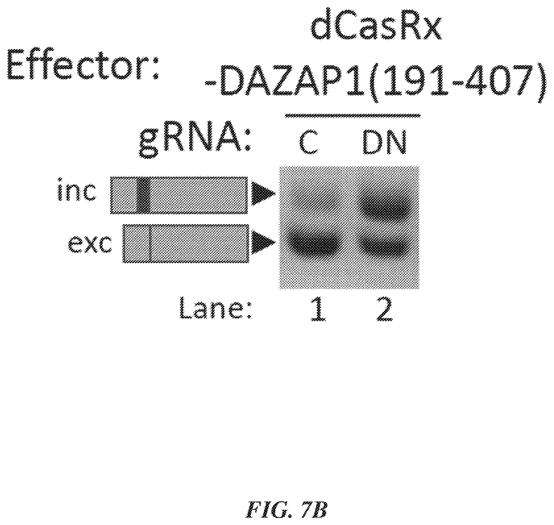

We tested the ability of DAZAP1 to induce exon inclusion when tethered by dCasRx to bind downstream of a cassette exon (FIG. 7A). We fused catalytic domain of DAZAP1 amino acids 191-407 to C-terminus of dCasRx [dCasRx-DAZAP1(191-407)] and directed it to downstream intron of SMN2-E7 by a mixture of three gRNAs (DN), and found that it could induce exon inclusion of SMN2-E7 (FIG. 7B, lane 2). Such activity was dependent on binding of dCasRx-DAZAP1(191-407) to the target RNA as non-targeting gRNA (C) did not induce exon inclusion (FIG. 7B, lane 1).

### Example 8. Tethering of U2 Auxiliary Factor (U2AF) to Introns Modulates Splicing

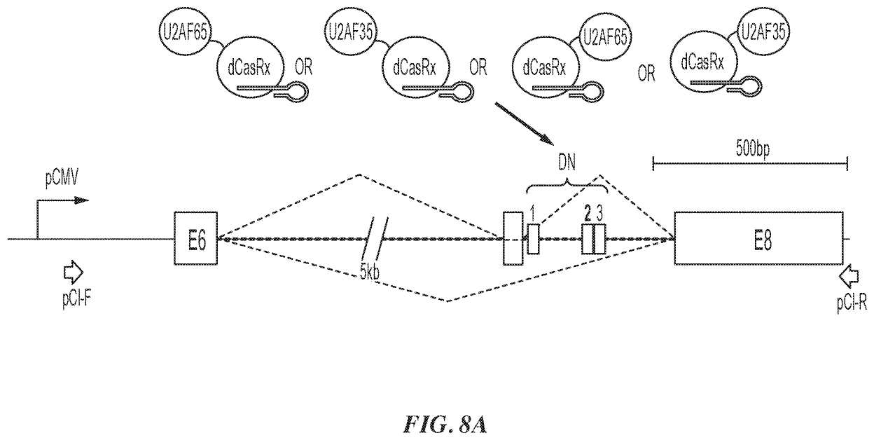

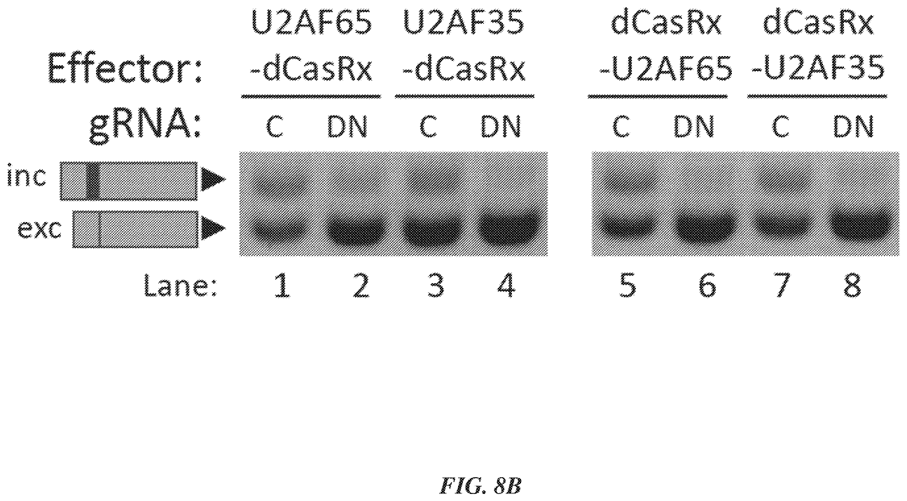

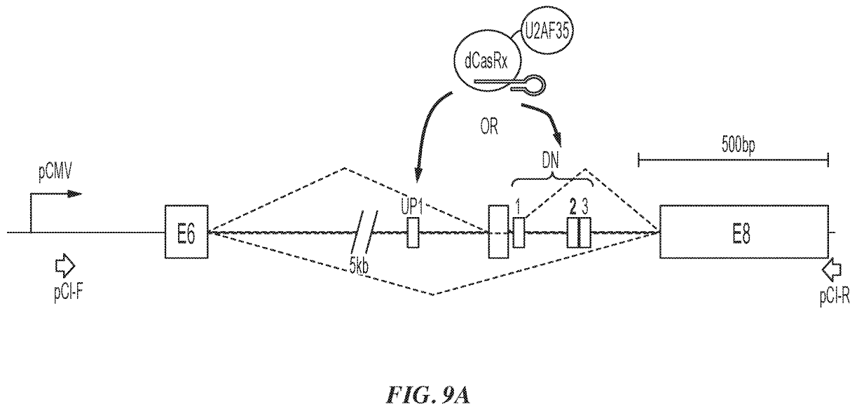

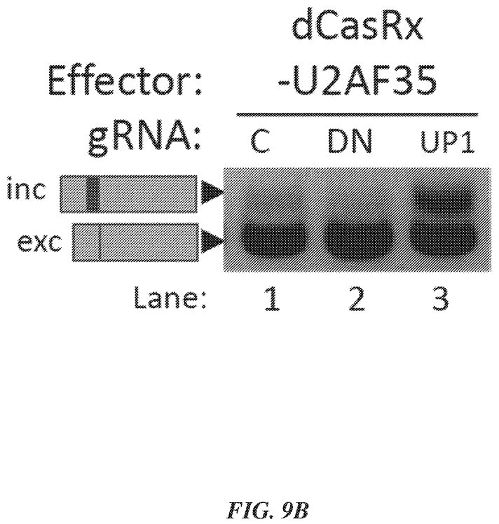

We fused two subunits of U2AF (U2AF65, U2AF35) separately to N- or C-termini of dCasRx to create four CRISPR Artificial Splicing factors (CASFx), U2AF65-dCasRx, U2AF35-dCasRx, dCasRx-U2AF65, dCasRx-U2AF35 and tested their activity when directed to bind at the intron downstream of SMN2-E7 (FIG. 8A). When directed by gRNAs to bind downstream of SMN2-E7, these CASFx induce exon exclusion (FIG. 8B, lanes 2,4,6,8). We next investigated whether a different effect would be induced if these CASFx were directed to bind to the intron upstream of SMN2-E7 (FIG. 9A). As shown in FIG. 9B, dCasRx-U2AF35 induced exon inclusion if bound to the intron upstream of SMN2-E7 (FIG. 9B, lane 3) while it induced exon exclusion if bound to the downstream intron (FIG. 9B, lane 2). This example demonstrates the targeting of CASFx to different sequence elements can induce different splicing effects on target RNAs.

### Example 9. Chemical-Inducible Exon Activation by Three-Component Two-Peptide iCASFx

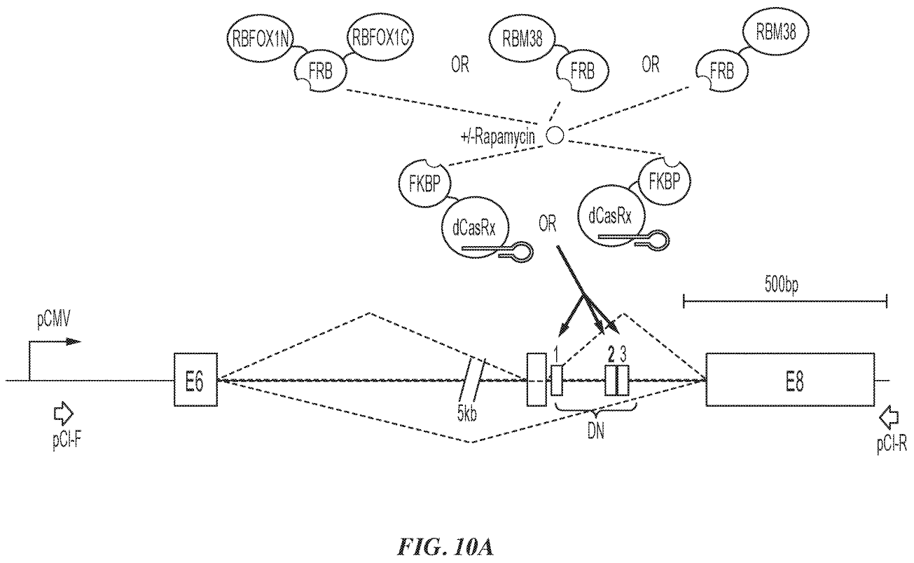

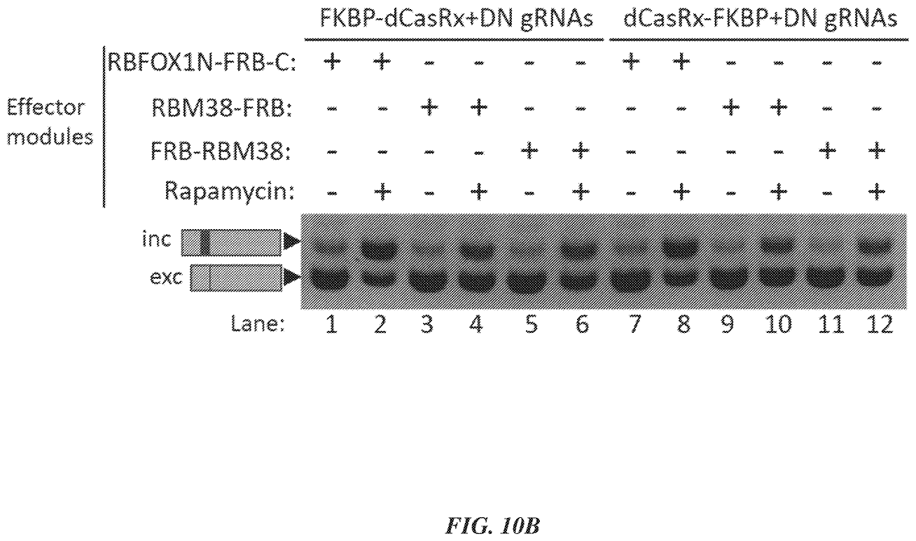

We created two-peptide inducible CRISPR Artificial Splicing Factors (iCASFx) by separating the RNA binding module (FKBP-dCasRx, or dCasRx-FKBP) and exon activation module (RBFOX1N-FRB-C, RBM38-FRB, or FRB-RBM38) into two peptides that can be induced to interact via the FKBP/FRB domains in the presence of rapamycin (FIG. 10A). As shown in FIG. 10B, cells cultured with rapamycin activated SMN2-E7 inclusion (FIG. 10B, lanes 2, 4, 6, 8, 10, and 12) compared to those without rapamycin (FIG. 10B, lanes 1, 3, 5, 7, 9, and 11). This example demonstrates that chemical-inducible CRISPR Artificial Splicing Factors iCASFx can be created by splitting the artificial splicing factor by chemical-inducible domains (e.g., FKBP/FRB).

### Example 10. Induction of Endogenous SMN2-E7 by RBFOX1N-dCasRx-C in GM03813 SMA2 Patient Fibroblast Cells

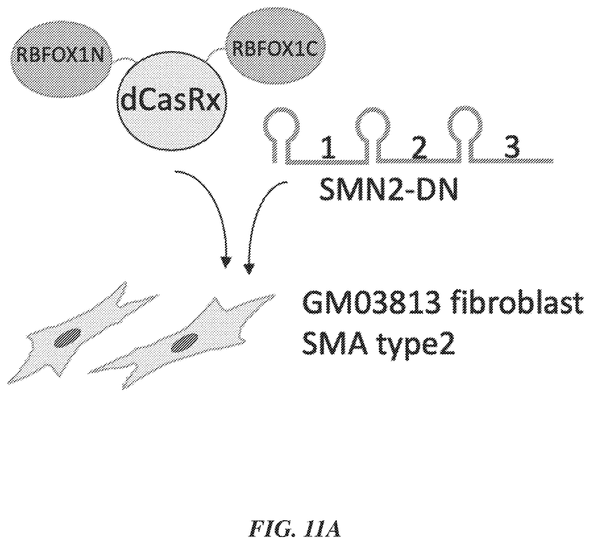

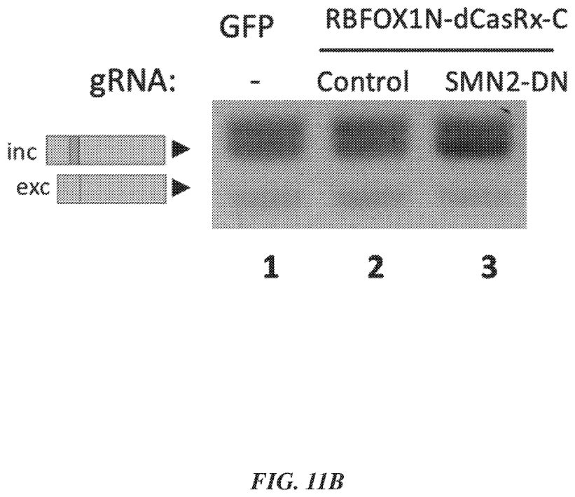

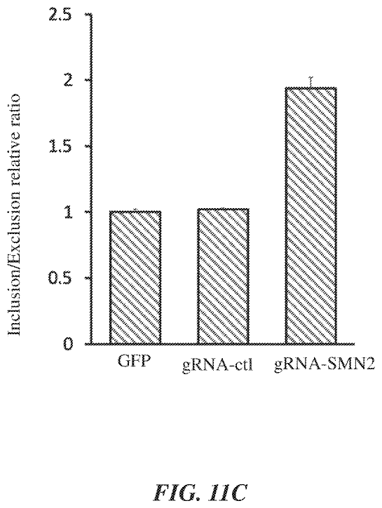

We tested the activation of endogenous SMN2-E7 exon by RBFOX1N-dCasRx-C in SMA2 patient cells by transfecting GM03813 cells (Coriell Institute) transiently with vectors expressing RBFOX1N-dCasRx-C and gRNA targeting downstream of SMN2-E7 (FIG. 11A). RBFOX1N-dCasRx-C and SMN2-DN gRNA in concert activated endogenous SMN2-E7 inclusion detected by both semi-quantitative RT-PCR (FIG. 11B) and quantitative RT-PCR (FIG. 11C).

### Example 11. Split CASFx (RBFOX1N-dCasRx-C) Architecture

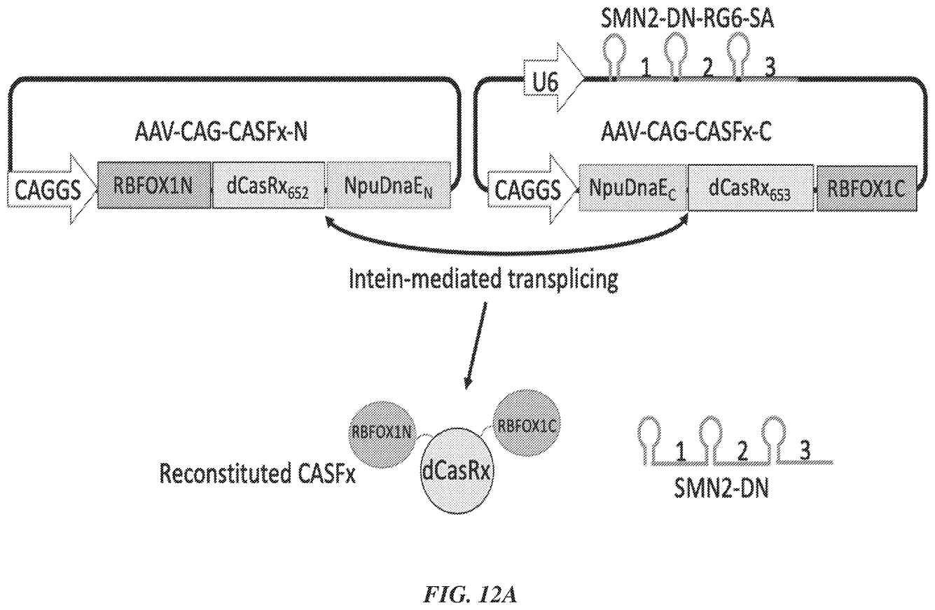

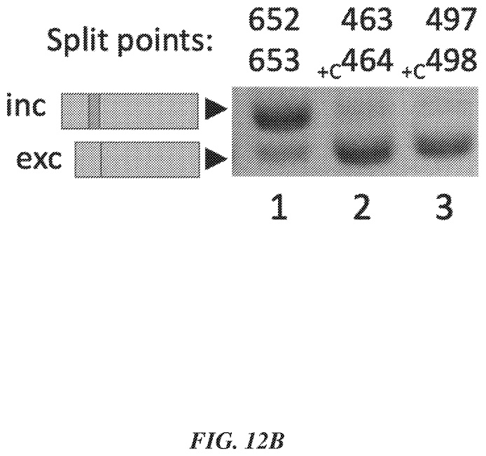

To fit CASFx into AAV vectors with limited payload, we split RBFOX1N-dCasRx-C into two fragments fused to split NpuDnaE intein elements. These split CASFx fragments were cloned into two separate AAV vectors with the C-split vectors carrying, in addition, the gRNA targeting SMN2 downstream intron (FIG. 12A). Three split designs were tested at different split points within the CasRx coding region, e.g., 652/653, 463/464, and 497/498. For split points 463/464 and 497/498, an obligatory cysteine for NpuDnaE splicing activity was added to the C-split fragment. Split RBFOX1N-dCasRx-C with the CasRx-652/653 split points supported SMN2-E7 exon activation detected by RT-PCR (FIG. 12B).

### Example 12. SNRPC-dCasRx Activates Splicing when Bound at Downstream Intron

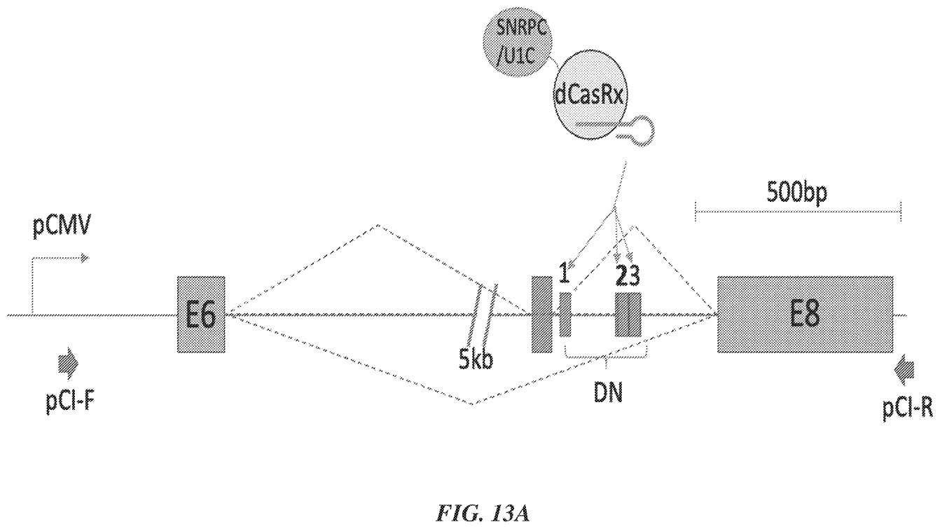

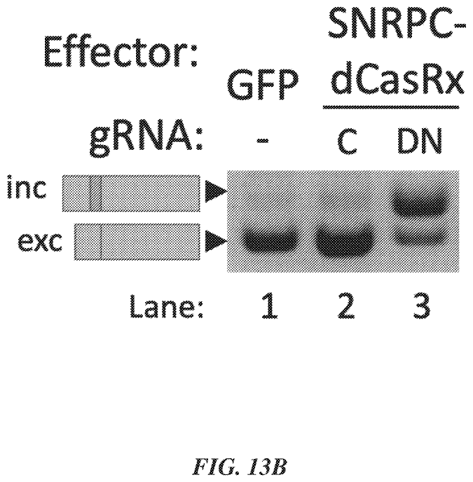

We tested the ability of core splicing factor SNRPC/U1C to induce exon inclusion when tethered by dCasRx to bind intron downstream of SMN2-E7 exon (FIG. 13A). We fused SNRPC to N-terminus of dCasRx [SNRPC-dCasRx] and directed it to downstream intron of SMN2-E7 by a mixture of three gRNAs (DN), and found that it could induce exon inclusion of SMN2-E7 (FIG. 13B, lane 3). Such activity was dependent on binding of SNRPC-dCasRx to the target RNA as non-targeting gRNA (C) did not induce exon inclusion (FIG. 13B, lane 1).

### Example 13. dNMCas9-RBM38 Activates Splicing when Bound at Downstream Intron

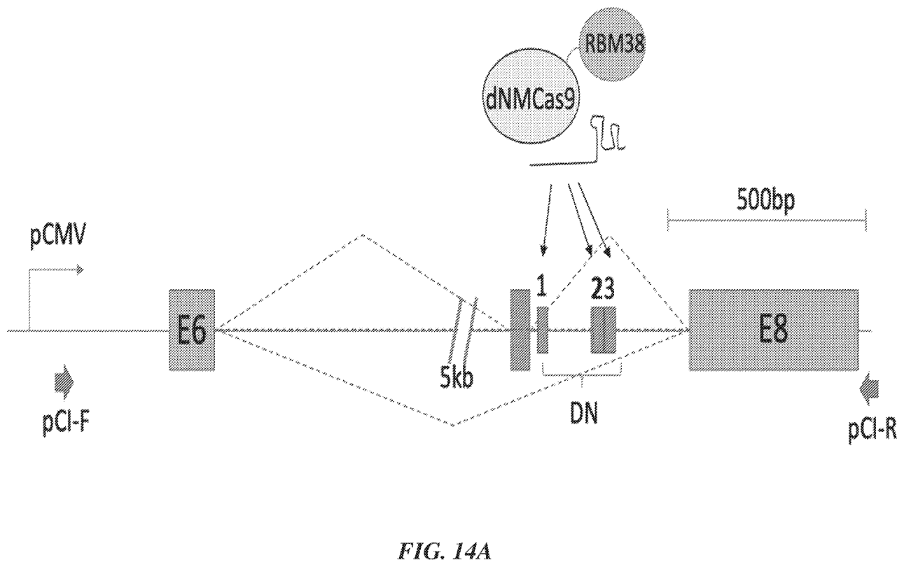

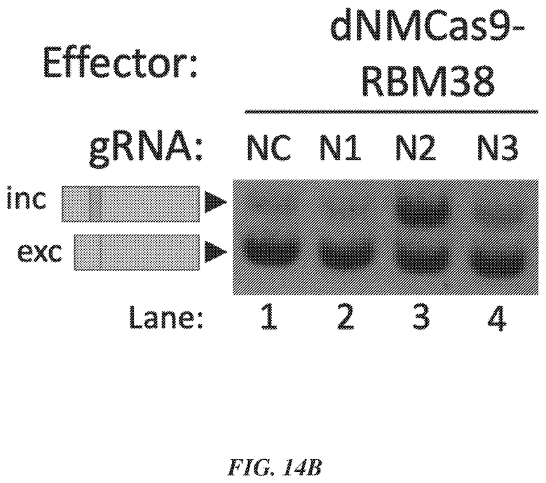

We tested the ability of dNMCas9 to tether RBM38 splicing factor to intron downstream of SMN2-E7 exon to activate its inclusion (FIG. 14A). We fused RBM38 to C-terminus of dNMCas9 [dNMCas9-RBM38] and directed it to downstream intron of SMN2-E7 by sgRNA N1, N2 or N3. dNMCas9-RBM38 directed by sgRNA-N2 induce exon inclusion of SMN2-E7 (FIG. 14B, lane 3). Such activity was dependent on binding of dNMCas9-RBM38 to the target RNA as non-targeting gRNA (NC) did not induce exon inclusion (FIG. 14B, lane 1).

### Materials and Methods

Cloning

HEK293T cDNA was used as a source for PCR-amplification of coding sequences of splicing factors or other RNA binding proteins. Alternatively, geneBlocks (gBlocks) encoding human codon optimized versions of their coding sequences were ordered from Integrated DNA Technologies (IDT; Coralville, Iowa USA) to serve as PCR template. The pXR002: EF1a-dCasRx-2A-EGFP plasmid (Addgene #109050) served as PCR template for dCasRx coding sequence. Coding sequence of a Neisseria meningitidis Cas9 (dNMCas9) was PCR-amplified from pHAGE-TO-dCas9-3×GFP (Addgene #64107). The coding sequences of the CRISPR Artificial Splicing Factors (CASFx) were then cloned into pmax expression vector (Lonza; Basel, Switzerland) by a combination of fusion PCR, restriction-ligation cloning and Sequence- and Ligation-Independent Cloning (SLIC) [DOI: 10.1128/AEM.00844-12] fusing the coding sequences splicing factors with those of dCasRx or dNMCas9 via polypeptide linkers. gRNA expression cloning plasmids were generated by similar procedures using IDT oligonucleotides encoding CasRx gRNA direct repeat and PCR reaction using a ccdbCam selection cassette (Invitrogen; Carlsbad, Calif. USA) and a U6-containing plasmid as templates. Two BbsI restriction sites flanking the ccdbCam selection cassette serves as the restriction cloning sites for insertion of target-specific spacers. Target-specific spacer sequences were then cloned into the gRNA expression plasmids by annealed oligonucleotide ligation.

To create the split CASFx constructs, fusion PCR was performed on gBlock encoding NpuDnaE inteins and N or C-terminal halves of CASFx (from pmax expression plasmid encoding the CASFx mentioned above) at different split points, followed by SLIC cloning into a Gateway donor plasmid, and subsequently recombined via LR clonase II Gateway recombination reaction into an AAV expression destination vector derived from AAV-CAG-GFP (Addgene #28014). Expression cassette encoding gRNA targeting intron downstream of SMN2-E7 were subsequently transferred to the AAV construct expression the C-split CASFx via PCR and SLIC.

Cell Culture and Transfection

For Examples 1-9 and 11-13, HEK293T cells were cultivated in Dulbecco's modified Eagle's medium (DMEM) (Sigma Aldrich; St. Louis, Mo. USA) with 10% fetal bovine serum (FBS)(Lonza; Basel Switzerland), 4% Glutamax (Gibco; Gaithersburg, Md. USA), 1% Sodium Pyruvate (Gibco; Gaithersburg, Md. USA) and penicillin-streptomycin (Gibco; Gaithersburg, Md. USA). Incubator conditions were 37° C. and 5% CO2. For activation experiments, cells were seeded into 12-well plates at 100,000 cells per well the day before being transfected with 600 ng (the “quota”) of plasmid DNA with 2.25 uL Attractene tranfection reagent (Qiagen; Hilden Germany). 18 ng of each reporter minigene plasmid was transfected. The remaining quota was then divided equally among the effector and gRNA plasmids. In cases where there were two or more gRNA plasmids, the quota allocated for gRNA plasmids is further subdivided equally. For two-peptide effectors (i.e., the MS2 and the FKBP-FRB systems), the effector plasmid quota was divided equally between the plasmids encoding the individual peptides. Media was changed 24 hr after transfection. 100 nM (final concentration) of rapamycin was added during media change if applicable. Cells were harvested 48 hr after transfection for RT-PCR analysis.

For Example 10, GM03813 fibroblasts derived from the SMA type II patient were obtained from Coriell Institute Cell Repository. Cells were maintained in Dulbecco's modified Eagle's medium (DMEM) (Sigma) with 10% fetal bovine serum (FBS) (Lonza), 4% Glutamax (Gibco), 1% Sodium Pyruvate (Gibco) and penicillin-streptomycin (Gibco). Incubator conditions were 37° C. and 5% CO2. CASFx plasmid with a GFP marker was nucleofected using 4D-Nucleofector™ System (Lonza) and the P2 Primary Cell 4D-Nucleofector kit (Lonza), program EN150. For each reaction, 1×10⁶ cells were collected, resuspended in 1000 complete P2 solution and mixed with plasmids DNA. GFP-positive cells were collected 2 days after nucleofection with FACSAria Fusion (BD Biosciences) and seeded in 6-well plate to expand. Cells pellets were collected 13 days after nucleofection for RNA extraction and downstream analysis.

RT-PCR

Cells were harvested for RNA extraction using RNeasy Plus Mini Kit (Qiagen; Hilden Germany). Equal amount of RNAs from one transfection experiment (either 700 ng or 1000 ng) were reverse-transcribed using High Capacity RNA-to-cDNA Kit (ThermoFisher; Waltham, Mass. USA). PCR was then performed using 2 uL (out of 10 uL) of cDNA using Phusion® High-Fidelity DNA Polymerase (New England Biolabs; Boston, Mass. USA) using minigene plasmid-specific primers for 25 cycles. PCR products were then analyzed on a 3% agarose gel.

Quantitative RT-PCR (qRT-PCR) for Endogenous SMN2-E7 Splicing Quantification in GM03813 Fibroblasts Cells.

Cells pellets were collected 13 days after nucleofection, and total RNA was isolated using RNeasy plus Mini Kit following the manufacturer's instructions (QIAGEN). 1 μg of RNA was used to synthesize cDNA using High Capacity RNA-cDNA kit (ThermoFisher Scientific) according to the supplier's protocol. qRT-PCR reaction was performed in a 20 μl mixture containing cDNA, primers, and 1×SYBR GREEN PCR Master mix (Roche). The following primers were used in the study:

Inclusion Isoform Forward Primer (SEQ ID NO: 55)

Inclusion Isoform Reverse Primer (SEQ ID NO: 56)

Exclusion Isoform Forward Primer (SEQ ID NO: 57)

Exclusion Isoform Reverse Primer (SEQ ID NO: 58)

### REFERENCES

1. Cech, T. R. & Steitz, J. A. The noncoding RNA revolution—trashing old rules to forge new ones. Cell 157, 77-94 (2014).
2. Glisovic, T., Bachorik, J. L., Yong, J. & Dreyfuss, G. RNA-binding proteins and post-transcriptional gene regulation. FEBS letters 582, 1977-1986 (2008).
3. Scotti, M. M. & Swanson, M. S. RNA mis-splicing in disease. Nature Reviews Genetics 17, 19 (2016).
4. Wang, Y., Cheong, C.-G., Hall, T. M. T. & Wang, Z. Engineering splicing factors with designed specificities. Nature methods 6, 825 (2009).
5. Bos, T. J., Nussbacher, J. K., Aigner, S. & Yeo, G. W. in RNA Processing 61-88 (Springer, 2016).
6. Abudayyeh, O. O. et al. C2c2 is a single-component programmable RNA-guided RNA-targeting CRISPR effector. Science 353, aaf5573 (2016).
7. Abudayyeh, O. O. et al. RNA targeting with CRISPR-Cas13. Nature 550, 280 (2017).
8. Konermann, S. et al. Transcriptome engineering with RNA-targeting type VI-D CRISPR effectors. Cell 173, 665-676. e614 (2018).
9. Orengo, J. et al. A bichromatic fluorescent reporter for cell-based screens of alternative splicing. Nucleic Acids Research 34, e148 (2006).

What is claimed is:

1. An artificial ribonucleic acid (RNA)-guided splicing factor comprising:
an RNA splicing factor linked to a catalytically inactive programmable nuclease.

2. The artificial RNA-guided splicing factor of claim 1, wherein the RNA splicing factor comprises an RNA-binding domain and a splicing domain.

3. The artificial RNA-guided splicing factor of claim 1 or 2, wherein the splicing factor is selected from RBFOX1, RBM38, DAZAP1, U2AF65, U2AF35, HNRNPH1, TRA2A, TRA2B, SYMPK, CPSF2, SRSF1, 9G8, PTB1/2, MBNL1/2/3, ESRP1, NOVA1, NOVA2, CELF4, SRM160, and SNRPC (U1C).

4. The artificial RNA-guided splicing factor of any one of claims 1-3, wherein the RNA splicing factor is fused to the catalytically inactive programmable nuclease.

5. The artificial RNA-guided splicing factor of claim 4, wherein the RNA splicing factor is fused to the amino terminus (N terminus) of the catalytically inactive programmable nuclease.

6. The artificial RNA-guided splicing factor of claim 4, wherein the RNA splicing factor is fused to the carboxy terminus (C terminus) of the catalytically inactive programmable nuclease.

7. The artificial RNA-guided splicing factor of any one of claims 1-6, wherein the catalytically inactive programmable nuclease is an RNA-guided Cas protein capable of binding RNA.

8. The artificial RNA-guided splicing factor of claim 7, wherein the catalytically inactive programmable nuclease is selected from catalytically inactive type VI-D CRISPR-Cas ribonucleases, C2c2/Cas13a ribonucleases, Cas13b ribonucleases, and a catalytically inactive Neisseria meningitidis Cas9 endonuclease.

9. The artificial RNA-guided splicing factor of claim 8, wherein the catalytically inactive type VI-D CRISPR-Cas ribonuclease is dCasRx.

10. The artificial RNA-guided splicing factor of any one of claims 1-9, wherein the catalytically inactive programmable nuclease comprises an N-terminal fragment of the catalytically inactive programmable nuclease linked to an N-terminal fragment of an intein and a C-terminal fragment of the catalytically inactive programmable nuclease linked to a C-terminal fragment of an intein, wherein the N-terminal fragment and the C-terminal fragment of the intein catalyze joining of the N-terminal and C-terminal fragments of the catalytically inactive programmable nuclease to produce the full-length artificial RNA-guided splicing factor.

11. The artificial RNA-guided splicing factor of any one of claims 1-10 bound to a guide RNA (gRNA).

12. A nucleic acid encoding the artificial RNA-guided splicing factor of any one of claims 1-10.

13. A recombinant viral genome comprising the nucleic acid of claim 12.

14. The recombinant viral genome of claim 13, wherein the recombinant viral genome is an AAV genome.

15. A viral particle comprising the recombinant viral genome of claim 13.

16. An AAV particle comprising the recombinant viral genome of claim 14.

17. A nucleic acid encoding an RNA splicing factor linked to an N-terminal fragment of a catalytically inactive programmable nuclease linked to an N-terminal fragment of an intein.

18. A nucleic acid encoding an RNA splicing factor linked to a C-terminal fragment of a catalytically inactive programmable nuclease linked to a C-terminal fragment of an intein.

19. A recombinant viral genome comprising the nucleic acid of claim 17 or 18.

20. The recombinant viral genome of claim 19, further encoding a gRNA.

21. The recombinant viral genome of claim 19 or 20, wherein the recombinant viral genome is an AAV genome.

22. A viral particle comprising the recombinant viral genome of claim 19 or 20.

23. An AAV particle comprising the recombinant viral genome of claim 21.

24. A composition comprising the artificial RNA-guided splicing factor of any one of claims 1-10 and a gRNA or a concatemer of tandem gRNAs.

25. The composition of claim 24, wherein the gRNA targets a first gene of interest.

26. The composition of claim 25, wherein the first gene of interest is SMN2.

27. The composition of claim 26, wherein the gRNA targets an intron between Exon 7 and Exon 8 of SMN2.

28. The composition of any one of claims 24-27, wherein the artificial RNA-guided splicing factor is complexed with the gRNA.

29. The composition of any one of claims 24-28, wherein the composition further comprises an additional gRNA that targets a second gene of interest.

30. The composition of claim 29, wherein the second gene of interest is a RG6 minigene.

31. The composition of claim 30, wherein the additional gRNA targets a splice acceptor site of the RG6 minigene.

32. A method of modulating RNA splicing, comprising
contacting a cell comprising a gene of interest with the artificial RNA-guided splicing factor of any one of claims 1-10 and a gRNA that targets RNA encoded by the gene of interest, and
inducing an exon inclusion and/or exclusion event in RNA encoded by the gene of interest.

33. A method of modulating RNA splicing, comprising
contacting a cell comprising two genes of interest with the artificial RNA-guided splicing factor of any one of claims 1-10 and a concatemer of tandem guide gRNAs, wherein one of the gRNAs targets RNA encoded by one of the genes of interest and the other of the gRNAs targets RNA encoded by the other of the genes of interest, and
inducing an exon inclusion event in RNA encoded by one of the genes of interest and inducing an exon exclusion event in RNA encoded by the other of the genes of interest.

34. A method of inducing an exon inclusion event, comprising
contacting a cell that expresses a gene of interest with the artificial RNA-guided splicing factor of any one of claims 1-10 and a guide RNA (gRNA) or a concatemer of tandem gRNAs that target(s) an intron adjacent to an exon of interest within RNA encoded by the gene of interest, and
inducing inclusion of the exon in the RNA encoded by the gene of interest.

35. The method of any one of claims 32-34, wherein the gene of interest is SMN2.

36. The method of claim 34, wherein the exon is Exon 7 of SMN2.

37. The method of claim 34, wherein the intron is located between Exon 7 and Exon 8 of SMN2.

38. The method of any one of claims 18-21, wherein the ratio of inclusion of the exon to exclusion of the exon and/or the ratio of exclusion of the exon to inclusion is increased by at least 1.5 fold, at least 2 fold, at least 5 fold, at least 10 fold, or at least 20 fold relative to a control.

39. A composition comprising an artificial RNA-guided splicing factor complex comprising:
a splicing factor modified to replace the RNA-binding domain with a first binding partner molecule;
a guide RNA modified to include a second binding partner molecule that is capable of binding to the first binding partner molecule; and
a catalytically inactive programmable nuclease.

40. A composition comprising:
a splicing factor modified to replace the RNA-binding domain with a first binding partner molecule; and/or
a guide RNA modified to include a second binding partner molecule that is capable of binding to the first binding partner molecule; and
optionally a catalytically inactive programmable nuclease.

41. The composition of claim 40 comprising a catalytically inactive programmable nuclease.

42. The composition of any one of claims 39-41, wherein the splicing factor is selected from RBFOX1, RBM38, DAZAP1, U2AF65, U2AF35, HNRNPH1, TRA2A, TRA2B, SYMPK, CPSF2, SRSF1, 9G8, PTB1/2, MBNL1/2/3, ESRP1, NOVA1, NOVA2, CELF4, SRM160, and SNRPC (U1C).

43. The composition of any one of claims 39-42, wherein the catalytically inactive programmable nuclease is an RNA-guided Cas protein capable of binding RNA.

44. The composition of claim 43, wherein the catalytically inactive programmable nuclease is selected from catalytically inactive type VI-D CRISPR-Cas ribonucleases, C2c2/Cas13a ribonucleases, Cas13b ribonucleases, and a catalytically inactive Neisseria meningitidis Cas9 endonuclease.

45. The composition of claim 44, wherein the catalytically inactive type VI-D CRISPR-Cas ribonuclease is dCasRx.

46. The composition of any one of claims 39-45, wherein the first binding partner molecule is a MS2 bacteriophage coat protein.

47. The composition of claim 46, wherein the second binding partner molecule is a stem-loop structure from the bacteriophage genome.

48. The composition of any one of claims 39-47, wherein the modified gRNA comprises at least two copies of the second binding partner molecule.

49. A method of modulating RNA splicing, comprising
contacting a cell comprising a gene of interest with (a) a splicing factor modified to replace the RNA-binding domain with a first binding partner molecule, (b) a guide RNA modified to include a second binding partner molecule that is capable of binding to the first binding partner molecule, and (c) a catalytically inactive programmable nuclease, wherein the gRNA targets RNA encoded by the gene of interest and
inducing an exon inclusion and/or exclusion event in the RNA encoded by the gene of interest.

50. A method of inducing an exon inclusion event, comprising
contacting a cell that expresses a gene of interest with (a) a splicing factor modified to replace the RNA-binding domain with a first binding partner molecule, (b) a guide RNA (gRNA) modified to include a second binding partner molecule that is capable of binding to the first binding partner molecule, and (c) a catalytically inactive programmable nuclease, wherein the gRNA targets an intron adjacent to an exon of interest within RNA encoded by the gene of interest, and
inducing inclusion of the exon in the RNA encoded by the gene of interest.

51. An artificial RNA-guided splicing factor complex comprising:
a first interaction domain fused to a catalytically inactive programmable nuclease;
a second interaction domain fused to splicing factor, wherein the first interaction domain and the second interaction domain dimerize in the presence of an inducer agent; and
a guide RNA.

52. The artificial RNA-guided splicing factor complex of claim 51, wherein the inducer agent is selected from a chemical agent, a biological agent, light, and heat.

53. The artificial RNA-guided splicing factor complex of claim 52, wherein the chemical agent is rapamycin, and optionally wherein the first and second interaction domain are selected from FRB protein and FKBP protein.

54. An artificial RNA-guided splicing factor complex comprising:
a first interaction domain fused to a catalytically inactive programmable nuclease;
a second interaction domain fused to splicing factor, wherein the first interaction domain and the second interaction domain are bound to an inducer agent; and
a guide RNA.

55. The artificial RNA-guided splicing factor complex of claim 54, wherein the inducer agent is a chemical agent.

56. The artificial RNA-guided splicing factor complex of claim 55, wherein the chemical agent is rapamycin, and optionally wherein the first and second interaction domain are selected from FRB protein and FKBP protein.

57. A composition comprising:
a first interaction domain fused to a catalytically inactive programmable nuclease;
a second interaction domain fused to splicing factor; and
a guide RNA,
wherein the first interaction domain and the second interaction domain bind to an inducer agent.

58. The composition of claim 57, wherein the inducer agent is a chemical agent.

59. The composition of claim 58, the chemical agent is rapamycin, and optionally wherein the first and second interaction domain are selected from FRB protein and FKBP protein.

60. A method of modulating RNA splicing, comprising:
contacting a cell that expresses a gene of interest with (a) a first interaction domain fused to a catalytically inactive programmable nuclease, (b) a second interaction domain fused to a splicing factor, and (c) a guide RNA, wherein the first interaction domain and the second interaction domain bind to an inducer agent, and wherein the gRNA targets RNA encoded by a gene of interest; and
inducing an exon inclusion and/or exon exclusion event in the RNA encoded by the gene of interest.

61. The composition of claim 60, wherein the inducer agent is a chemical agent.

62. The composition of claim 61, the chemical agent is rapamycin, and optionally wherein the first and second interaction domain are selected from FRB protein and FKBP protein.
